(19) United States
(12) Patent Application Publication
(10) Pub. No.: US 2023/0250478 A1
(43) Pub. Date: Aug. 10, 2023

(54) DIAGNOSIS AND TREATMENT OF CHRONIC DIABETIC COMPLICATIONS USING LONG NONCODING RNAS AS TARGETS

(71) Applicant: THE UNIVERSITY OF WESTERN ONTARIO, London (CA)

(72) Inventors: Subrata CHAKRABARTI, London (CA); Saumik BISWAS, London (CA); Shali CHEN, London (CA); Biao FENG, London (CA)

(73) Assignee: THE UNIVERSITY OF WESTERN ONTARIO, London, ON (CA)

(21) Appl. No.: 18/014,322

(22) PCT Filed: Jul. 6, 2021
(86) PCT No.: PCT/CA2021/050924

§ 371 Date: Jan. 3, 2023

Related U.S. Application Data
(60) Provisional application No. 63/048,389, filed on Jul. 6, 2020.

(51) Int. Cl. C12Q 1/6883; C12N 15/113; A61K 31/706; C12Q 1/686; A61P 27/02

(52) U.S. Cl. CPC C12Q 1/6883; C12N 15/113; A61K 31/706; C12Q 1/686; A61P 27/02; C12Q 2600/118; C12Q 2600/178

(57) ABSTRACT

Method of identifying a subject as having an increased likelihood of progressing to end-organ damage due to diabetes comprising comparing the amount of a lncRNA in a sample from the subject with a reference value, and if the amount of the lncRNA in the sample is increased relative to the reference value, identifying the subject as having an increased probability of progressing to end-organ damage due to diabetes. The lncRNA is one or more of HOTAIR, H19, WISPER, ZFAS1, HULC, ANRIL, MALAT1, MIAT, and MEG3. Also methods of treating chronic diabetic complications and other conditions comprising administering a HOTAIR inhibitor.

### FIELD OF THE INVENTION

The present invention pertains generally to long non-coding RNAs (lncRNAs) and methods of using them diagnostically and therapeutically. In particular, the invention relates to lncRNAs and their uses in treatment of chronic diabetic complications.

### BACKGROUND OF THE INVENTION

With diabetes mellitus (DM) projected to impact over 600 million people globally in the next 20 years (1), the subsequent risk of acquiring micro- and macrovascular complications remain a serious concern. DM is a degenerative metabolic disease that is primarily characterized by chronic hyperglycemia, where sustained hyperglycemic insults can evoke alterations in biochemical and metabolic pathways that ultimately contribute to vascular damage and the pathogenesis of chronic diabetic complications (2). Among these complications, diabetic retinopathy (DR) is a debilitating microvascular complication of DM and is also one of the leading causes of blindness worldwide (3). Although there are different severity stages for DR, DR can be mainly categorized as non-proliferative DR (NPDR) and proliferative DR (PDR), where the latter may lead to imminent vision loss due to the presence of ocular pathological neovascularization (4).

As a result of long-term diabetes, pathological angiogenesis occurs and continuously activates various signal transduction cascades that promote the expressions of several pro-angiogenic genes, leading to increased concentrations of angiogenic factors than angiostatic factors (5). Following upregulation, these angiogenic factors act synergistically to mediate the migration and proliferation of retinal endothelial cells in pre-existing blood vessels, which ultimately leads to the formation of new, abnormal blood vessels that are susceptible to bleeding, leakage, fibrosis and contraction (6). Among the regulatory angiogenic molecules, vascular endothelial growth factor (VEGF) is a potent angiogenic factor expressed by ECs and non-ECs and has been studied extensively in DR. Several pathological processes, such as hypoxia (7), oxidative stress (8), advanced glycation end products (9), and inflammation (10), can stimulate VEGF expressions through a transcriptional regulation involving a complex milieu of transcription factors (11) and mediator complexes (12). Indeed, due to VEGF's critical role in DR, the standard first-line therapy for patients with diabetic macular edema and PDR consists of intravitreal injections of anti-VEGF and/or steroid compounds, which temporarily delay the progression of severe retinopathy. However, the use of such therapies comes at cost to patients, where frequent intraocular injections are required, local or systemic adverse effects are associated with anti-VEGF compounds (13,14), and 40-50% of eyes with diabetic macular edema (another complication of diabetes) cannot fully respond to anti-VEGF treatments (15). Undoubtedly, in order to mitigate the effects of DR, a sense of urgency is warranted for better diagnosis and targeted therapies.

Within the last two decades, the rapid advent of genomic technologies has identified long non-coding RNAs (lncRNAs) as a fundamental class of RNA transcripts that are larger than 200 base-pairs and possess limited protein-coding capacities. LncRNAs are dynamically regulated and present with distinct functionalities that facilitate chromatin remodelling and/or help govern the expression of genes involved in a multitude of biological and pathological processes, including development (16), cancer (17), and neurodegeneration (18).

Thus, there remains a need in the art for identifying and characterizing lncRNAs that can be used in developing diagnostics and therapeutics.

### SUMMARY OF THE INVENTION

This invention relates to long non-coding RNAs (lncRNAs) and their diagnostic, prognostic, and therapeutic uses for conditions such as diabetic retinopathy (DR). In particular, the invention relates to lncRNAs that play roles in regulation of genes involved in diabetes-induced angiogenesis. Such lncRNAs can be used as biomarkers to diagnose DR, including early stage DR. One of the identified lncRNAs, HOTAIR, mediates high glucose (HG) induced angiogenesis and the expressions of angiogenesis-promoting and diabetes-associated genes. Inhibitors of HOTAIR expression can be used in the treatment of chronic diabetic complications such as DR, including early stage DR as well as other conditions and disease associated with HOTAIR expression described in this invention.

In one embodiment, this invention provides for a method of identifying a subject as having an increased likelihood of progressing to end-organ damage due to diabetes comprising: a) measuring the amount of a long non-coding RNA (lncRNA) in a biological sample derived from a subject; and b) comparing the amount of the lncRNA in the sample with a reference value, and if the amount of the lncRNA in the sample is increased or decreased relative to the reference value, identifying the subject as having an increased likelihood of progressing to end-organ damage due to diabetes, wherein the lncRNA is one or more of HOTAIR, H19, WISPER, ZFAS1, HULC, ANRIL, MALAT1, MIAT, and MEG3.

In one embodiment, if the amount of the lncRNA HOTAIR in the sample indicates that the subject has an increased likelihood of progressing to end-organ damage due to diabetes, the method further comprises treating the subject for the end-organ damage due to diabetes.

In another embodiment the method further comprises administering to the subject a therapeutically effective amount of an agent that inhibits at least one biological activity of lncRNA HOTAIR if the amount of the lncRNA HOTAIR in the sample indicates that the subject has an increased likelihood of progressing to end-organ damage due to diabetes.

In one embodiment, the amount of lncRNA is done by performing polymerase chain reaction (PCR) using at least one set of oligonucleotide primers comprising a forward primer and a reverse primer capable of amplifying a lncRNA polynucleotide sequence, wherein at least one set of primers selected from: when the subject is a human a forward primer comprising the sequence of SEQ ID NOs: 3, 77, 79, 81, 83, 85, 87, 89, 91 or 93 and a corresponding reverse primer comprising the sequence of SEQ ID NO: 4, 78, 80, 82, 84, 86, 88, 90, 92 or 94, and when the subject is a mouse a forward primer comprising the sequence of SEQ ID NO:43 and a reverse primer comprising the sequence of SEQ ID NO:44.

In another embodiment, the biological sample is serum or vitreous fluid.

In another embodiment, the present invention is a method of diagnosing diabetic retinopathy (DR) in a subject, the method comprising: a) measuring the amount of a long non-coding RNA (lncRNA) in a biological sample derived from the subject; and b) comparing the amount of the lncRNA with a control reference value, and when the amount of the lncRNA is altered relative to the control reference value, diagnosing the subject as having DR, wherein the lncRNA is one or more of HOTAIR, H19, WISPER, ZFAS1, HULC, ANRIL, MALAT1, MIAT, and MEG3.

In one embodiment of the method of diagnosing DR in a subject, when the subject is diagnosed with DR, the method further comprises treating the subject for DR.

In another embodiment of the method of diagnosing DR in a subject, when the subject is diagnosed with DR, the method further comprises administering to the subject a therapeutically effective amount of an agent that inhibits at least one biological activity of lncRNA HOTAIR.

In another embodiment of the method of diagnosing DR in a subject, the amount of lncRNA is measured by performing polymerase chain reaction (PCR) using at least one set of oligonucleotide primers comprising a forward primer and a reverse primer capable of amplifying a lncRNA polynucleotide sequence, wherein at least one set of primers selected from: when the subject is a human a forward primer comprising the sequence of SEQ ID NOs: 3, 77, 79, 81, 83, 85, 87, 89, 91 or 93 and a corresponding reverse primer comprising the sequence of SEQ ID NO: 4, 78, 80, 82, 84, 86, 88, 90, 92 or 94, and when the subject is a mouse a forward primer comprising the sequence of SEQ ID NO:43 and a reverse primer comprising the sequence of SEQ ID NO:44.

In another embodiment of the method of diagnosing DR in a subject, the biological sample is serum or vitreous fluid.

In one embodiment, the present invention provides for a method of treating a subject of DR, the method comprising: a) measuring the amount of the long non-coding RNA HOTAIR in a biological sample derived from the subject; b) analyzing the amount of the long non-coding RNA HOTAIR in conjunction with respective reference value ranges for the long non-coding RNA HOTAIR, wherein an increased amount of the long non-coding RNA HOTAIR in the biological sample compared to a control sample indicates that the subject has DR; and c) administering to the subject in need thereof a therapeutically effective amount of an agent that inhibits at least one biological activity of the long non-coding RNA HOTAIR if the amount of the long non-coding RNA HOTAIR indicates that the subject has DR.

In one embodiment, measuring the amount of the long non-coding RNA HOTAIR in a biological sample comprises performing polymerase chain reaction (PCR) with at least one set of oligonucleotide primers comprising a forward primer and a reverse primer capable of amplifying a HOTAIR long non-coding RNA polynucleotide sequence, wherein at least one set of primers is selected from the group consisting of: when the subject is a human: a forward primer comprising the sequence of SEQ ID NOs: 3, 77, 79, 81, 83, 85, 87, 89, 91 or 93 and a corresponding reverse primer comprising the sequence of SEQ ID NO: 4, 78, 80, 82, 84, 86, 88, 90, 92 or 94 and when the subject is a mouse: a forward primer comprising the sequence of SEQ ID NO: 43 and a reverse primer comprising the sequence of SEQ ID NO: 44.

In another embodiment, the present invention provides for a method of treating a condition, the method comprising: administering to a subject in need thereof a therapeutically effective amount of at least one agent that inhibits at least one biological activity of the long non-coding RNA HOTAIR. In embodiments, the condition associated with HOTAIR expression is one or more of:

diabetic retinopathy (DR), diabetic nephropathy, diabetic cardiomyopathy, diabetic neuropathy, proliferative vitreoretinopathies, neovascular glaucoma, ischemic retinopathy, retinopathy secondary to retinal vein occlusion, as well as age-related macular degeneration, keloid formation, and wound healing. In one aspect, the condition is DR. In another aspect the DR is non-proliferative DR or proliferative DR.

In another embodiment, the present invention provides for a method of treating a condition in which anti-VEGF therapy is ineffective, the method comprising: administering to the subject in need thereof a therapeutically effective amount of at least one agent that inhibits at least one biological activity of the long non-coding RNA HOTAIR.

In another embodiment, the present invention provides for a method of treating a patient who does not respond to anti-VEGF therapy, the method comprising: administering to the subject in need thereof a therapeutically effective amount of at least one agent that inhibits at least one biological activity of the long non-coding RNA HOTAIR.

In another embodiment, the present invention provides for a method of preventing glucose-induced oxidative damage, the method comprising: administering to a subject in need thereof a therapeutically effective amount of at least one agent that inhibits at least one biological activity of the long non-coding RNA HOTAIR.

In another embodiment, the present invention provides for a method of preventing the induction of epigenetic mediators in hyperglycemic environments, the method comprising inhibiting the expression of lncRNA HOTAIR.

In another embodiment the present invention provides for a use of at least one agent that inhibits at least one biological activity of the long non-coding RNA HOTAIR for treating a condition, wherein the condition is one or more of: diabetic retinopathy, diabetic nephropathy, diabetic cardiomyopathy, diabetic neuropathy, proliferative vitreoretinopathies, neovascular glaucoma, ischemic retinopathy, retinopathy secondary to retinal vein occlusion, age-related macular degeneration, keloid formation, and wound healing. In one aspect the condition is diabetic retinopathy. In another aspect, the diabetic retinopathy is non-proliferative diabetic retinopathy or proliferative diabetic retinopathy.

In another embodiment, the present invention provides for a use of at least one agent that inhibits at least one biological activity of the long non-coding RNA HOTAIR for treating a condition in which anti-VEGF therapy is ineffective.

In another embodiment, the present invention provides for a use of at least one agent that inhibits at least one biological activity of the long non-coding RNA HOTAIR for preventing glucose-induced oxidative damage.

In another embodiment, the present invention provides for a use of at least one agent that inhibits at least one biological activity of the long non-coding RNA HOTAIR for preventing the induction of epigenetic mediators in hyperglycemic environments.

In one embodiment of the present invention, the epigenetic mediator is EZH2, SUZ12, EED, DNMT1, DNMT3A, DNMT3B, CTCF or P300.

In another embodiment of the present invention, the at least one agent is one or more of an anti-long non-coding RNA HOTAIR antibody or an antibody fragment.

In another embodiment of the present invention, the at least one agent is one or more a siRNA, piRNA, snRNA a miRNA, a ribozyme, or an antisense oligonucleotide.

In another embodiment of the present invention, the subject is a human.

In another embodiment of the present invention, the at least one agent is the siRNA, wherein the siRNA is SEQ ID NO:104, SEQ ID NO:106, SEQ ID NO:108, SEQ ID NO:110, N-187951-01, 187951-02, 187951-03, 187951-04.

In another embodiment of the present invention, the at least one agent is a DNMT inhibitor or a histone methylation inhibitor.

In another embodiment of the present invention, the DNMT inhibitor is 5-aza-dC or siDNMT1.

In another embodiment of the present invention, the histone methylation inhibitor is DZNep and/or siEZH2.

In another embodiment of the present invention, the agent is administered in combination with another therapeutic agent for treating the condition associated with diabetes-induced neovascularization, such as an anti-VEGF agent.

In another embodiment, the present invention provides for a method of treating a disease, comprising administering an amount of a siRNA in an amount effective to treat the disease, wherein the siRNA is SEQ ID NOs: 104, 106, 108 or 110.

In another embodiment, the present invention relates to an isolated siRNA selected from SEQ ID NOs: 104, 106, 108 and 110.

### BRIEF DESCRIPTION OF THE DRAWINGS

The following figures illustrate various aspects and preferred and alternative embodiments of the invention.

FIGS. 1A-1C. LncRNA microarray findings from HRECs cultured in NG or HG for 48 hours (GEO: GSE122189). (A) Scatter plot demonstrates lncRNA expressions between normal glucose (NG) and high glucose (HG) replicates. In general, the scatter plot is a visualization method used for assessing the lncRNA expression variation (or reproducibility) between the two compared samples (or groups). The values of X and Y axes in the scatter plot are the normalized signal values (log 2 scaled) of two samples or the averaged normalized signal values (log 2 scaled) for two groups. The green lines are fold change lines (the default fold change value given is 2.0). The lncRNAs above the top green line and below the bottom green line indicates more than 2.0 fold change of lncRNAs between the two compared groups or samples. (B) Hierarchical clustering for the lncRNAs in all sample groups. “Red” indicates high relative expression, and “blue” indicates low relative expression. (C) Venn diagrams depicting the total number of lncRNAs that were upregulated (top) or downregulated (bottom) between NG and HG replicates.

FIGS. 2A-2D. HOTAIR RNA expressions are associated with increased expressions of angiogenic markers in HRECs cultured with high glucose (HG) and appear to be glucose-dependent with significant elevations at 48 hours. RT-qPCR analyses demonstrating HG-induced increases of (A) HOTAIR, (B) VEGF-A, and (C) ET-1 in HRECs compared to HRECs cultured in basal glucose levels at 48 hours (normal glucose; NG). (D) Relative HOTAIR RNA levels following different glucose concentrations following 48 hours of culture. β-actin was used as an internal control. Statistical significance was assessed using two-tailed Student's t-test when comparing two conditions or one-way ANOVA for multiple comparisons followed by Tukey's post hoc test (*p<0.05 or **p<0.01). Data represents the mean±SEM of 3 independent experiments (n=6/group).

FIGS. 3A-3B. High glucose promotes HOTAIR expressions and HOTAIR can be localized in the nucleus and cytoplasm of retinal endothelial cells. (A) Visualization of HOTAIR localization in HRECs at 48 hours as indicated by RNA fluorescence in situ hybridization using Stellaris FISH probes for human HOTAIR with Qasar 570 dye. Cells were also counterstained with DAPI to visualize the nuclei. Original magnification, 20×; scale bars, 100 μm. (B) Mean integrated densities of HOTAIR expressions calculated using ImageJ. Statistical significance was assessed using two-tailed Student's t-test (****p<0.0001). Data represents the mean±SEM of 50 cells captured per sample (n=4-5 independent samples/group).

FIGS. 4A-4C. HOTAIR directly mediates angiogenesis in vitro. (A) Images captured from the endothelial tube formation assay at the 6-hour mark for HRECs treated with scrambled siRNAs (SCR), siHOTAIR or exogenous VEGF proteins and cultured in NG or HG conditions. The WimTube Image analyzer software was used to calculate (B) the number of tubules and (C) the total branching points in each group. Statistical significance was assessed using one-way ANOVA for multiple comparisons, followed by Tukey's post hoc test (****p<0.0001). Data represents the mean±SEM of 3 independent experiments (n=8/group) and images were captured from at least two field views per well. Original magnification, 40×.

FIG. 5. HOTAIR knockdown using three different commercially available siRNAs. HRECs were pre-treated with scrambled (SCR) siRNAs or specific siRNAs targeting HOTAIR prior to NG or HG culture for 48 hours. RT-qPCR was then used to analyze the expressions of HOTAIR. actin was used as an internal control. Statistical significance was assessed using one-way ANOVA for multiple comparisons, followed by Tukey's post hoc test (****p<0.0001). Data represents the mean±SEM of 3 independent experiments (n=6/group).

FIGS. 6A-6I. HOTAIR knockdown can prevent the induction of several angiogenic factors and diabetes-related molecules in vitro. RT-qPCR analyses of (A) HOTAIR, (B) VEGF-A, (C) ET-1, (D) ANGPTL4, (E) PGF, (F) (G) HIF-1α, (H) PARP1, and (I) Cytochrome B expressions following the administration of SCR siRNA or siHOTAIR in HRECs subjected to 48 hours of NG or HG culture. β-actin was used as an internal control. Statistical significance was assessed using one-way ANOVA for multiple comparisons, followed by Tukey's post hoc test (*p<0.05, **p<0.01, ***p<0.001, ****p<0.0001, or n.s.=not significant). Data represents the mean±SEM of 3 independent experiments (n=6/group).

FIGS. 7A-7H. HOTAIR knockdown can prevent the induction of several epigenetic mediators in hyperglycemic environments. RT-qPCR analyses of (A) EZH2, (B) SUZ12, (C) EED, (D) DNMT1, (E) DNMT3A, (F) DNMT3B, (G) CTCF, and (H) P300 expressions following the administration of SCR siRNA or siHOTAIR in HRECs subjected to 48 hours of NG or HG culture. β-actin was used as an internal control. Statistical significance was assessed using one-way ANOVA for multiple comparisons, followed by Tukey's post hoc test (*p<0.05, **p<0.01, ***p<0.001, ****p<0.0001, or n.s.=not significant). Data represents the mean±SEM of 3 independent experiments (n=6/group).

FIGS. 8A-8D. HOTAIR knockdown can reduce VEGF-A proteins, improve cellular viability, and prevent glucose-induced decreases of HOXD loci in HRECs. (A) VEGF-A ELISA results (expressed as pg/mL) from HRECs that were pre-treated with SCR siRNA or siHOTAIR and subjected to NG or HG culture for 48 hours. (B,C) RT-qPCR analyses of HOXD3 and HOXD10 expressions following HOTAIR knockdown. β-actin was used as an internal control. (D) WST-1 findings for SCR or siHOTAIR-treated HRECs. Statistical significance was assessed using one-way ANOVA for multiple comparisons, followed by Tukey's post hoc test (*p<0.05, **p<0.01, ***p<0.001, ****p<0.0001, or n.s.=not significant). Data represents the mean±SEM of 3 independent experiments (n=6/group).

FIGS. 9A-9B. Hotair is significantly elevated in the retinas of diabetic animals at 2 months. Non-diabetic (control) and streptozotocin-induced diabetic C57BL/6J mice or Sprague-Dawley rats were followed for 2 months. Retinal tissues were isolated and extracted for RNA. RT-qPCR was employed to analyze retinal Hotair expressions in (A) mice and (B) rats. β-actin was used as an internal control. Statistical significance was assessed using the Mann-Whitney U test. Data represents the mean±SD (n=8 per control or diabetic mice group, n=5 per control rat group or n=9 per diabetic rat group; *p<0.05).

FIGS. 10A-10F. SiRNA-mediated knockdown of mus Hotair and its impact on angiogenic markers in mouse retinal and lung endothelial cells. RT-qPCR analyses of (A,D) Hotair, (B,E) Vegf-a, and (C,F) Angptl4 expressions following the administration of SCR siRNA or siHOTAIR in mouse retinal endothelial cells (top panel) and primary mouse lung endothelial cells (bottom panel) subjected to 48 hours of NG or HG culture. β-actin was used as an internal control. Statistical significance was assessed using one-way ANOVA for multiple comparisons, followed by Tukey's post hoc test (*p<0.05, **p<0.01, ***p<0.001, ****p<0.0001, or n.s.=not significant). Data represents the mean±SEM of 3 independent experiments (n=6/group).

FIGS. 11A-11E. Hematoxylin and eosin (H&E) staining of various mouse tissues following siHOTAIR toxicology experiments. Wild-type C57BL/6 mice were subjected to a one-time intravitreal injection that consisted of either scrambled siRNA control (100 nM; SCR) or siHOTAIR at varying concentrations (25 nM, 50 nM, and 100 nM) and were monitored for seven days and then euthanized for tissue collection (n=3 per group). No behavioural changes or ocular complications were observed in the mice throughout the duration of the experiment and as evidenced by H&E staining, no cellular abnormalities were also observed across (A) retinal, (B) heart, (C) lung, (D) liver, and (E) kidney tissues following the one-time intravitreal siHOTAIR injection at 25, 50 or 100 nM concentrations (images not shown for 25 nM). Original magnification, 40×; scale bar=5 microns.

FIGS. 12A-12C. In vivo results following the knockdown of Hotair. (A) Relative Hotair knockdown expressions, as indicated by RT-qPCR, in the retinal tissues of C57BL/6J mice from our toxicology experiments involving different siHOTAIR concentrations (n=3 per group). β-actin was used as an internal control. (B,C) Body weights and blood glucose levels of all C57BL/6J mice involved in our short-term, one-month therapeutic model, where intravitreal injections of scrambled (SCR; 100 nM) siRNAs or siHOTAIR (100 nM) were administered to non-diabetic and diabetic mice eyes once every week for up to three weeks. Statistical significance was assessed using one-way ANOVA for multiple comparisons, followed by Tukey's post hoc test (****p<0.0001 or n.s.=not significant). Data represents the mean±SD (n=6/group).

FIGS. 13A-13O. In vivo knockdown of Hotair can significantly prevent early glucose-induced elevations of angiogenic and diabetes-associated molecules in the diabetic retina. Non-diabetic and diabetic C57BL/6J mice were administered intravitreal injections of scrambled siRNAs (SCR) or siHOTAIR once every week for up to 3 weeks. Animals were then euthanized at 4 weeks (1 month) and retinal tissues were isolated and extracted for RNA. RT-qPCR was employed to analyze (A) Hotair, (B) Vegf-a, (C) Et-1, (D) Angptl4, (E) Parp1, (F) Mcp-1, (G) Il-1β, (H) p300, (I) Ezh2, (J) Suz12, (K) Eed, (L) Pgf, (M) Hif-1α, (N) Ctcf, and (O) Hoxd3 expressions. ƒt-actin was used as an internal control. Statistical significance was assessed using one-way ANOVA for multiple comparisons, followed by Tukey's post hoc test (*p<0.05, **p<0.01, ***p<0.001, ****p<0.0001, or n.s.=not significant). Data represents the mean±SD (n=6/group).

FIGS. 14A-14G. Hematoxylin and eosin (H&E) staining of various mouse tissues following our short-term, 1-month therapeutic animal model involving siHOTAIR. Non-diabetic (control) and diabetic C57BL/6 mice were subjected to intravitreal injections of scrambled (SCR; 100 nM) siRNAs or siHOTAIR (100 nM) once every week for up to three weeks. Mice were monitored throughout the duration of the experiment and subsequently euthanized for tissue collection at 4 weeks (n=3 per group). Similar to our initial toxicology experiments, no behavioural changes or ocular complications were observed in the mice and as evidenced by H&E staining, no cellular abnormalities were also observed across (A) retinal, (B) heart, (C) lung, (D) liver, (E) kidney, (F) cortical, and (G) hippocampal tissues following multiple intravitreal siHOTAIR injections at 1 month. Original magnification, 40×; scale bar=5 microns.

FIGS. 15A-15C. HOTAIR is upregulated in the serum and vitreous of patients with proliferative diabetic retinopathy (PDR). RT-qPCR analyses were used to examine HOTAIR expressions in the (A) vitreous and (B) serum from non-PDR (control) and PDR patients. β-actin was used as an internal control. Statistical significance was assessed using the Mann-Whitney U test. Data represents the mean±SD (n=10 per control group or n=11 per PDR group; **p<0.01 or ****p<0.0001). (C) Two-sided Pearson correlations determined that a linear (positive) association for HOTAIR expressions existed between the two sample types (***p<0.001).

FIGS. 16A-16B. HOTAIR knockdown can partially prevent glucose-induced mitochondrial depolarization/dysfunction. (A) Images captured from the JC-1 assay, where HRECs were pre-treated with scrambled (SCR) siRNAs or siHOTAIR and subsequently cultured in NG or HG for 48 hours. Mitochondrial depolarization is indicated by more green and less red fluorescence (low ΔΨM, suggesting unhealthy/dysfunctional mitochondrial), while a polarized mitochondrial state is indicated by more red and less green fluorescence (normal to high ΔΨM, suggesting healthy/functional mitochondria). Cells were also counterstained with DAPI to visualize the nuclei. Original magnification, 20×; scale bars, 100 μm. (B) JC-1 red/green fluorescence ratio was calculated using ImageJ. Statistical significance was assessed using one-way ANOVA for multiple comparisons, followed by Tukey's post hoc test (*p<0.05, **p<0.01, ***p<0.001, ****p<0.0001, or n.s.=not significant). Data represents the mean±SEM of 20 cells captured per sample (n=8 independent samples/group).

FIGS. 17A-17B. Knockdown of HOTAIR can significantly prevent glucose-induced oxidative damage. (A) Images captured from the 8-OHdG assay, where HRECs were pre-treated with scrambled (SCR) siRNAs or siHOTAIR and subsequently cultured in NG or HG for 48 hours. 8-OHdG is a biomarker for nuclear and mitochondrial oxidative DNA damage, where heightened oxidative damage is indicated by strong green fluorescence. Cells were also counterstained with DAPI to visualize the nuclei. Original magnification, 20×; scale bars, 100 μm. (B) Mean integrated densities of 8-OHdG expressions calculated using ImageJ. Statistical significance was assessed using one-way ANOVA for multiple comparisons, followed by Tukey's post hoc test (*p<0.05 or ****p<0.0001). Data represents the mean±SEM of 20 cells captured per sample (n=8 independent samples/group).

FIGS. 18A-18B. HOTAIR knockdown can prevent glucose-induced disruptions of endothelial cell junctions in vitro. Representative images, by electron microscopic detection, of (A) scrambled siRNA (SCR) or (B) siHOTAIR-transfected HRECs after high glucose culture (n=6 samples per group). Disruptions of endothelial cell junctions can be visualized in SCR plus HG cells compared to preservation of junctions in siHOTAIR plus HG cells (a higher magnification was inserted in B showing the junctions [indicated by the black arrows]). Direct magnification, 1950×; scale bar=2 microns; ‘N’=nucleus.

FIGS. 19A-19I. Glucose metabolism regulates HOTAIR and most of its downstream targets in vitro. RT-qPCR analyses of (A) HOTAIR, (B) VEGF-A, (C) ET-1, (D) ANGPTL4, (E) MCP-1, (F) IL1β, (G) CTCF, (H) Cytochrome B, and (I) PARP1 expressions following 2-deoxy-D-glucose treatment (0.6 or 5 mM) in HRECs subjected to 48 hours of NG (5 mM D-glucose) or HG (25 mM D-glucose) culture. 2-deoxy-D-glucose is a potent inhibitor of glycolysis. β-actin was used as an internal control. Statistical significance was assessed using one-way ANOVA for multiple comparisons, followed by Tukey's post hoc test (*p<0.05, **p<0.01, ***p<0.001, ****p<0.0001, or n.s.=not significant). Data represents the mean±SEM of 3 independent experiments (n=6/group).

FIGS. 20A-20I. Glycolytic inhibition can impact certain epigenetic molecules and may also influence nuclear transport molecules involved in oxidative stress, independent of the mitochondria. RT-qPCR analyses of (A) EZH2, (B) SUZ12, (C) EED, (D) P300, (E) DNMT1, (F) DNMT3A, (G) DNMT3B, (H) HOXD3, and (I) HOXD10 expressions following 2-deoxy-D-glucose treatment (0.6 or 5 mM) in HRECs subjected to 48 hours of NG (5 mM D-glucose) or HG (25 mM D-glucose) culture. 2-deoxy-D-glucose is a potent inhibitor of glycolysis. β-actin was used as an internal control. Statistical significance was assessed using one-way ANOVA for multiple comparisons, followed by Tukey's post hoc test (*p<0.05, **p<0.01, ***p<0.001, ****p<0.0001, or n.s.=not significant). Data represents the mean±SEM of 3 independent experiments (n=6/group).

FIGS. 21A-21E. DZNep pre-treatment reduces the expression of PRC2 components and stimulates the transcription of HOXD loci. HRECs were pre-treated with DZNep (a global histone methylation inhibitor) prior to NG or HG culture for 48 hours. RT-qPCR was then used to analyze the expressions of (A) EZH2, (B) SUZ12, (C) EED, (D) HOXD3, and (E) HOXD10. actin was used as an internal control. Statistical significance was assessed using one-way ANOVA for multiple comparisons, followed by Tukey's post hoc test (*p<0.05, **p<0.01, ***p<0.001, ****p<0.0001, or n.s.=not significant). Data represents the mean±SEM of 3 independent experiments (n=6/group).

FIGS. 22A-22J. Histone methylation differentially regulates HOTAIR and its downstream targets. HRECs were pre-treated with DZNep (a global histone methylation inhibitor) prior to NG or HG culture for 48 hours. RT-qPCR was then used to analyze the expressions of (A) HOTAIR, (B) VEGF-A, (C) ET-1, (D) ANGPTL4, (E) CTCF, (F) PARP1, (G) MCP-1, (H) IL-1β, (I) P300, and (J) Cytochrome B. β-actin was used as an internal control. Statistical significance was assessed using one-way ANOVA for multiple comparisons, followed by Tukey's post hoc test (*p<0.05, **p<0.01, ***p<0.001, ****p<0.0001, or n.s.=not significant). Data represents the mean±SEM of 3 independent experiments (n=6/group).

FIGS. 23A-23L. EZH2 and CTCF are directly involved in the transcriptional regulation of HOTAIR and several other downstream genes. RT-qPCR analyses of (A) HOTAIR, (B) VEGF-A, (C) ET-1, (D) ANGPTL4, (E) EZH2, (F) CTCF, (G) SUZ12, (H) EED, (I) PARP1, (J) MCP-1, (K) IL-1β, and (L) Cytochrome B expressions following the administration of scrambled (SCR) siRNAs, siEZH2, or siCTCF in HRECs subjected to 48 hours of NG or HG culture. EZH2 is the catalytic subunit of PRC2 (a critical histone methyltransferase) and CTCF is an important epigenetic transcription factor involved in the direct regulation of genes. β-actin was used as an internal control. Statistical significance was assessed using one-way ANOVA for multiple comparisons, followed by Tukey's post hoc test (*p<0.05, **p<0.01, ***p<0.001, ****p<0.0001, or n.s.=not significant). Data represents the mean±SEM of 3 independent experiments (n=6/group).

FIGS. 24A-24F. The knockdown of EZH2 and CTCF can alter glucose-induced expressions of certain epigenetic molecules. RT-qPCR analyses of (A) DNMT1, (B) DNMT3A, (C) DNMT3B, (D) P300, (E) HOXD3, and (F) HOXD10 expressions following the administration of scrambled (SCR) siRNAs, siEZH2, or siCTCF in HRECs subjected to 48 hours of NG or HG culture. EZH2 is the catalytic subunit of PRC2 (a critical histone methyltransferase) and CTCF is an important epigenetic transcription factor involved in the direct regulation of genes. β-actin was used as an internal control. Statistical significance was assessed using one-way ANOVA for multiple comparisons, followed by Tukey's post hoc test (*p<0.05, **p<0.01, ***p<0.001, ****p<0.0001, or n.s.=not significant). Data represents the mean±SEM of 3 independent experiments (n=6/group).

FIGS. 25A-25B. High glucose promotes strong binding associations between HOTAIR and epigenetic enzymes. RNA immunoprecipitation (RIP) experiments were conducted using anti-IgG, anti-EZH2 (catalytic subunit of the histone methyltransferase, PRC2), or anti-P300 (a histone acetyltransferase) antibodies on HRECs cultured with NG or HG for 48 hours. RT-qPCR was then used to determine the fold enrichment of HOTAIR following IgG, (A) EZH2 and (B) P300 pulldown. IgG antibodies were used as a negative control and β-actin was used as an internal control. Statistical significance was assessed using one-way ANOVA for multiple comparisons, followed by Tukey's post hoc test (****p<0.0001 or n.s.=not significant). Data represents the mean±SEM of 3 independent experiments (n=3/group).

FIGS. 26A-26F. HOTAIR can govern the transcriptional status of VEGF-A in hyperglycemic environments. ChIP-qPCR analyses examining the enrichment of (A,D) RNA polymerase II (Pol II), (B,E) tri-methylation of lysine 27 in histone 3 (H3K27me3; a repressive histone mark), and (C,F) acetylation of lysines 9, 14, 18, 23, and 27 in histone 3 (H3K9/14/18/23/27; an active histone mark) in the distal (top panel) and proximal (bottom panel) regions of VEGF-A. In order to determine the role of HOTAIR in transcriptional regulation, HRECs were pre-treated with scrambled (SCR) siRNAs or siHOTAIR and subsequently cultured in NG or HG for 48 hours prior to ChIP-qPCR experimentation. IgG antibodies were used as a negative control and β-actin was used as an internal control. Statistical significance was assessed using one-way ANOVA for multiple comparisons, followed by Tukey's post hoc test (****p<0.0001 or n.s.=not significant). Data represents the mean±SEM of 3 independent experiments (n=3/group).

FIGS. 27A-27B. DNA methylation profiling of HRECs. (A) Information on the human HOTAIR gene according to the UCSC database, where the position of HOTAIR is located on chromosome 12: 54,356,092-54,368,740 (hg19) and its approximate size (for transcript variant 1) is 12,649 nucleotides containing a total of 6 exons (19). (B) Unsupervised hierarchical clustering with heatmap using all the CpGs in HRECs that span across HOTAIR, which amounted to 59 probes. Interestingly, there are no distinctions between cells treated with different concentrations of glucose (5 mM versus 25 mM) and the duration of culture (2 versus 7 days)—alluding to the stable epigenetic nature of DNA methylation in these cells following glucose treatment for different durations. Rows indicated CpGs and columns show the samples; the color scale from blue to red indicates the level of methylation from zero to one (with zero indicating no methylation and one indicating maximum methylation; n=3 independent samples per group indicated by the top panel colors; HR5_2=HRECs cultured in 5 mM glucose for 2 days, HR25_2=HRECs cultured in 25 mM glucose for 2 days, HR5_7=HRECs cultured in 5 mM glucose for 7 days, and HR25_7=HRECs cultured in 25 mM glucose for 7 days.

FIGS. 28A-28B. DNA methylation profiling of HRECs. (A) Panel that depicts the stable DNA methylation patterns across the HOTAIR genomic regions shared between HRECs cultured with NG or HG for various durations (2 or 7 days). (B) Differential methylation patterns in the HOTAIR promoter region. The box plots represent the distribution of median methylation values across all of the probes mapping to this region as stratified by glucose concentration and culture duration. Center line: median of regional methylation levels across samples; lower and upper bounds: first and third quartiles; whiskers: interquartile ranges (n=3 independent samples per group).

FIGS. 29A-29E. 5-aza-dC can decrease the expressions of DNMTs, while promoting HOXD3 and HOXD10 gene expressions. HRECs were pre-treated with 5-aza-dC (a pan-DNMT inhibitor) prior to NG or HG culture for 48 hours. RT-qPCR was then used to analyze the expressions of (A) DNMT1, (B) DNMT3A, (C) DNMT3B, (D) HOXD3, and (E) HOXD10. β-actin was used as an internal control. Statistical significance was assessed using one-way ANOVA for multiple comparisons, followed by Tukey's post hoc test (*p<0.05, **p<0.01, ***p<0.001, ****p<0.0001, or n.s.=not significant). Data represents the mean±SEM of 3 independent experiments (n=6/group).

FIGS. 30A-30J. Global inhibition of DNMTs can differentially regulate the expressions of HOTAIR and its targets. HRECs were pre-treated with 5-aza-dC (a pan-DNMT inhibitor) prior to NG or HG culture for 48 hours. RT-qPCR was then used to analyze the expressions of (A) HOTAIR, (B) VEGF-A, (C) ET-1, (D) ANGPTL4, (E) CTCF, (F) P300, (G) PARP1, (H) Cytochrome B, (I) MCP-1, and (J) IL-1β. β-actin was used as an internal control. Statistical significance was assessed using one-way ANOVA for multiple comparisons, followed by Tukey's post hoc test (*p<0.05, **p<0.01, ***p<0.001, ****p<0.0001, or n.s.=not significant). Data represents the mean±SEM of 3 independent experiments (n=6/group).

FIGS. 31A-31E. SiRNA-mediated knockdown of DNMT1 can influence glucose-induced expressions of DNMTs and HOXD loci. RT-qPCR analyses of (A) DNMT1, (B) DNMT3A, (C) DNMT3B, (D) HOXD3, and (E) HOXD10 expressions following the administration of scrambled (SCR) siRNAs or siDNMT1 in HRECs subjected to 48 hours of NG or HG culture. DNMT1 is a constitutively expressed DNA methyltransferase. β-actin was used as an internal control. Statistical significance was assessed using one-way ANOVA for multiple comparisons, followed by Tukey's post hoc test (*p<0.05, **p<0.01, ***p<0.001, ****p<0.0001, or n.s.=not significant). Data represents the mean±SEM of 3 independent experiments (n=6/group).

FIGS. 32A-32J. Selective knockdown of DNMT1 can impact the expressions of HOTAIR and some of its downstream targets in vitro. RT-qPCR analyses of (A) HOTAIR, (B) VEGF-A, (C) ET-1, (D) ANGPTL4, (E) CTCF, (F) P300, (G) Cytochrome B, (H) PARP1, (I) MCP-1, and (J) IL-1β expressions following the administration of scrambled (SCR) siRNAs or siDNMT1 in HRECs subjected to 48 hours of NG or HG culture. DNMT1 is a constitutively expressed DNA methyltransferase. β-actin was used as an internal control. Statistical significance was assessed using one-way ANOVA for multiple comparisons, followed by Tukey's post hoc test (*p<0.05, **p<0.01, ***p<0.001, ****p<0.0001, or n.s.=not significant). Data represents the mean±SEM of 3 independent experiments (n=6/group).

FIG. 33: Schematic of the cell culture model used in the present examples.

FIG. 34: HG-treated HRECs demonstrate differential expressions of lncRNAs at 48 hours. RT-qPCR analysis of ANRIL, H19, HOTAIR, HULC, MALAT1, MEG3, MIAT, WISPER, and ZFAS1 expressions in HRECs exposed to 25 mM (HG) or 5 mM (NG) glucose over 48 hours [data (mean±SEM); N=6 per group; normalized to β-actin, and expressed as a fold change of NG; *=p<0.05 compared to NG].

FIG. 35. General map of the AAV-siRNA-GFP vector provided by ABM. Both U6 and H1 promoters initiate transcription of the siRNA and the PolyT sequence at the end of the siRNA stops transcription. This convergent transcription system works at the RNA level and does not go through translation where the start and stop codons are needed. From this, transcription of both the sense and anti sense sequence form complementary RNAs, which would anneal and form a double stranded siRNA, which bypasses the formation of a hairpin loop and would be used to form the RNA-induced silencing complex.

FIG. 36. DNA sequencing chromatogram. The siRNA sequence is highlighted, with the polyT sequence following downstream of the siRNA.

FIGS. 37A-37C. A single intravitreal dose of AAV2-siHOTAIR can significantly reduce retinal Hotair levels in the retinal tissues of diabetic animals at 1 month. Non-diabetic and diabetic C57BL/6J mice were administered either no injection or a single intravitreal injection of PBS (sham control) or AAV2-siHOTAIR. Animals were then euthanized at 4 weeks (1 month) and retinal tissues were isolated and extracted for RNA. RT-qPCR was employed to analyze (A) Hotair, (B) Angptl4, and (C) Vegfa expressions. β-actin was used as an internal control. Statistical significance was assessed using one-way ANOVA for multiple comparisons, followed by Tukey's post hoc test (*p<0.05 when compared to other groups). Data represents the mean±SEM (n=4/group).

FIGS. 38A-38I. Differential expressions of lncRNAs in the serum of patients. Serum samples were obtained from patients prior to undergoing vitrectomy. RNA was isolated using TRIzol reagent and then reverse transcribed to cDNA, where RT-qPCR was used to determine the expressions of the target lncRNAs. Following RT-qPCR, we subsequently performed linear regression analyses using the lncRNA expression profiles. As shown above, there appear to be significant relationships between PDR and the expressions of A) HOTAIR, B) ANRIL, C) H19, D) HULC, E) MALAT1, H) WISPER and I) ZFAS1. Although we did not find a significant relationship between PDR and F) MEG3 or G) MIAT, we anticipate that an increase in sample size may help improve the significance of this relationship. Legend: The black dots indicate control patients without PDR and the black squares indicate patients with confirmed PDR. The dotted line is for the linear regression, while the equation in each graph is the result of the regression analysis including the slope and intersection. We have also provided the R² value, which is the coefficient of determination [p<0.05 was considered significant; N=10 for control group and N=11 for PDR group; DR=PDR; and normalized to β-actin].

FIGS. 39A-39I. Differential expressions of lncRNAs in the vitreous fluid of patients. Vitreous samples were obtained from patients undergoing vitrectomy. RNA was isolated using TRIzol reagent and then reverse transcribed to cDNA, where RT-qPCR was used to determine the expressions of the target lncRNAs. Following RT-qPCR, we subsequently performed linear regression analyses using the lncRNA expression profiles. As shown above, there appear to be significant relationships between PDR and the expressions of A) HOTAIR, B) ANRIL, D) MALAT1, F) MIAT, G) WISPER, H) ZFAS1 and I) H19. Although we did not find a significant relationship between PDR and C) HULC or E) MEG3, we anticipate that an increase in sample size may help improve the significance of this relationship. Legend: The black dots indicate control patients without PDR and the black squares indicate patients with confirmed PDR. The dotted line is for the linear regression, while the equation in each graph is the result of the regression analysis including the slope and intersection. We have also provided the R² value, which is the coefficient of determination [p<0.05 was considered significant; N=10 for control group and N=12 for PDR group; DR=PDR; and normalized to β-actin].

FIGS. 40A-40I. Pearson correlation analyses between serum and vitreous samples. When comparing between serum and vitreous samples, significant correlations were observed for A) HOTAIR, B) ANRIL, C) H19, E) MALAT1, H) WISPER and I) ZFAS1, which suggests that the expressions of these lncRNAs can be reflected from the serum and vitreous of patients with PDR. Although we did find significant correlations between serum and vitreous concentrations of D) HULC, F) MEG3, and G) MIAT, including a larger sample size may further help confirm the relationships between these markers and sample types [p<0.05 was considered significant; N=10 for control group and N=11 for PDR group; DR=PDR; and normalized to β-actin].

FIGS. 41A-41D. Custom double-stranded siRNAs. Four different siRNAs were developed that targeted various regions of HOTAIR (Table 7). HRECs were transfected with these siRNAs and then HOTAIR and its target markers were subsequently analyzed. RT-qPCR analyses of A) HOTAIR, B) VEGF-A, C) ET-1, and D) ANGPTL4 indicate that the knockdown of HOTAIR can directly influence the expressions of these angiogenic transcripts, albeit variable reductions existing for each siRNA. In particular, compared to SCR HG controls, HOTAIR expressions were reduced by ˜67%, ˜41%, ˜57%, and ˜32% using siHOTAIR SB1, siHOTAIR SB2, siHOTAIR SB3, and siHOTAIR SB4, respectively. Statistical significance was assessed using one-way ANOVA for multiple comparisons, followed by Tukey's post hoc test (*p<0.05, **p<0.01, ***p<0.001, ****p<0.0001, or n.s.=not significant). Data represents the mean±SEM (n=3/group). SCR=scrambled siRNA; SB=siRNAs custom designed; NG=normal/basal glucose; HG=high glucose.

FIG. 42. Illustration showing serum lncRNA expressions are distinct in diabetic patients (with varying stages of diabetic retinopathy) when compared to non-diabetic patients (without diabetic retinopathy). Illustration showing significant relationships between control patients (without DR) and diabetic patients with varying stages of DR were observed for the lncRNAs ANRIL, H19, HOTAIR, HULC, MALAT1, MIAT, WISPER, and ZFAS1 [p<0.05 was considered significant, indicated by “#”; N=4 for control group and N=38 for DR group; and lncRNA expressions were normalized to β-actin]. Although not significant, MEG3 levels demonstrated increasing trends in diabetic patients compared to control patients (p=0.063).

FIG. 43. Illustration showing lncRNA expressions based on the various stages of diabetic retinopathy. When comparing between serum lncRNAs and their expression levels across various stages of diabetic retinopathy (‘0’=patients with diabetes and no DR, ‘1’=patients with diabetes and non-proliferative DR, and ‘2’=patients with diabetes and proliferative DR) against control patients (without diabetes and DR), significant expressions were observed for only H19 in group ‘0’, all 9 lncRNAs in group ‘1’ and 7 lncRNAs in group ‘2’ (ANRIL, H19, HOTAIR, HULC, MALAT1, MIAT, and WISPER) when compared to the control group [p<0.05 was considered significant, indicated by “#”; N=4 for control group and N=8 for the “0” group, N=12 for the “1” group, N=18 for the “2” group; and lncRNA expressions were normalized to β-actin].

### DETAILED DESCRIPTION

The practice of the present invention will employ, unless otherwise indicated, conventional methods of pharmacology, chemistry, biochemistry, recombinant DNA techniques and immunology, within the skill of the art. Such techniques are explained fully in the literature. See, e.g., Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (3rd Edition, 2001); Methods In Enzymology (S. Colowick and N. Kaplan eds., Academic Press, Inc.).

All publications, patents and patent applications cited herein, whether supra or infra, are hereby incorporated by reference in their entireties.

### I. Definitions

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “an lncRNA” includes a mixture of two or more lncRNAs, or a plurality of lncRNAs, unless the context clearly is to the contrary, and so forth. The term “plurality” as used herein means “one or more.”

Throughout this specification and the claims, the terms “comprise,” “comprises,” and “comprising” are used in a non-exclusive sense, except where the context requires otherwise. Likewise, the terms “include”, “has” and their grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range.

The term “about,” particularly in reference to a given quantity, is meant to encompass deviations of plus or minus five percent.

“HOTAIR” (for HOX transcript antisense RNA) refers to a long non-coding RNA transcript produced from a human gene located on chromosome 12 (spanning from 54,356,092 to 54,368,740 nucleotides (GRCh37/hg19)). Following transcription, splicing, and polyadenylation, HOTAIR transcripts do not encode for proteins, since these transcripts contain little or no open reading frames (GENBANK IDs: NR_047517.1, NR_003716.3, NR_047518.1).

The VEGF-A (Vascular endothelial growth factor A) gene is a member of the PDGF/VEGF growth factor family. It encodes a heparin-binding protein, which exists as a disulfide-linked homodimer. This growth factor induces proliferation and migration of vascular endothelial cells, and is essential for both physiological and pathological angiogenesis. Indeed, VEGF-A is known as an important contributor to the development of diabetic macular edema and proliferative DR [20].

ET-1 (Endothelin-1) also known as preproendothelin-1 (PPET1), is a potent vasoconstrictor in humans that is encoded by the EDN1 gene and produced by vascular endothelial cells. In addition to its vasoconstrictive properties, ET-1 can also act as a mitogen on vascular smooth muscle and may ultimately play a role in the development of vascular diseases [21]. Indeed, ET-1 is upregulated in organs affected by chronic diabetic complications and contributes to the development of DR [22].

ANGPTL4 refers to a gene that encodes for Angiopoietin-like 4 protein, which is implicated in the metastatic process by modulating vascular permeability, cancer cell motility and invasiveness. Additionally, ANGPTL4 has been shown to be a potent angiogenic mediator in proliferative DR [23].

PGF (Placental Growth Factor) is a protein that in humans is encoded by the PGF gene. Placental growth factor is a member of the VEGF sub-family—a key molecule in angiogenesis and vasculogenesis, in particular during embryogenesis. Furthermore, PGF has been implicated in the pathogenesis of DR [24].

IL-1β (Interleukin 1 beta), also known as leukocytic pyrogen, leukocytic endogenous mediator, mononuclear cell factor, lymphocyte activating factor and other names, is a cytokine protein that in humans is encoded by the IL1B gene. IL-1β activity contributes to a heightened inflammatory environment and can also be involved in early angiogenic responses induced by tumour cells [25, 26]. In the context of DR, IL-1β concentrations are found at elevated concentrations in the vitreous of proliferative DR patients and has well-documented pathogenic implications in DR [27, 28, 29].

HIF-1α, Hypoxia-inducible factor 1-alpha, is a subunit of a heterodimeric transcription factor hypoxia-inducible factor 1 (HIF-1) that is encoded by the HIF1A gene. It is a basic helix-loop-helix PAS domain containing protein, and is considered as the master transcriptional regulator of cellular and developmental response to hypoxia. The dysregulation and overexpression of HIF1A by either hypoxia or genetic alternations have been heavily implicated in cancer biology, as well as a number of other pathophysiologies, specifically in areas of vascularization and angiogenesis, energy metabolism, cell survival, and tumor invasion. Moreover, increased intravitreal concentrations of HIF-la have been documented in diabetic patients with proliferative DR and are mutually related with VEGF levels [30].

PARP1 (Poly [ADP-ribose] polymerase 1) is a gene that encodes in humans for the enzyme PARP-1 also known as NAD+ ADP-ribosyltransferase 1 or poly[ADP-ribose] synthase 1. PARP1 is one of the PARP family of enzymes. PARP activation is a downstream effector of oxidant-induced DNA damage and a critical step in functional and metabolic changes in tissues affected by diabetes [31]. PARP1 can also be involved in the transcriptional regulation of pertinent DR-associated molecules, such as MMP-9 [32].

Cytochrome B refers to a protein found in the mitochondria of eukaryotic cells. It functions as part of the electron transport chain and is the main subunit of transmembrane cytochrome bc1 and b6f complexes. Elevated Cytochrome B levels have been identified in various cells impacted by hyperglycemia and such glucose-induced increases are indicative of mitochondrial dysfunction, which can further contribute to the pathogenesis of diabetic complications [33, 34].

Diabetes-induced conditions that can be treated or diagnosed according to embodiments presented herein include, non-proliferative and proliferative diabetic retinopathy, neovascular glaucoma, ischemic retinopathy, and diabetic complications such as diabetic nephropathy, diabetic cardiomyopathy, and diabetic neuropathy.

The terms “siRNA” and “short interfering RNA” are interchangeable and refer to single-stranded or double-stranded RNA molecules that are capable of inducing RNA interference. SiRNA molecules typically have a duplex region that is between 18 and 30 base pairs in length.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide.

The term “homologous region” refers to a region of a nucleic acid with homology to another nucleic acid region. Thus, whether a “homologous region” is present in a nucleic acid molecule is determined with reference to another nucleic acid region in the same or a different molecule. Further, since a nucleic acid is often double-stranded, the term “homologous, region,” as used herein, refers to the ability of nucleic acid molecules to hybridize to each other. For example, a single-stranded nucleic acid molecule can have two homologous regions which are capable of hybridizing to each other. Thus, the term “homologous region” includes nucleic acid segments with complementary sequence.

The term “complementary” and “complementarity” are interchangeable and refer to the ability of polynucleotides to form base pairs with one another. 100% complementary refers to the situation in which each nucleotide unit of one polynucleotide strand or region can hydrogen bond with each nucleotide unit of a second polynucleotide strand or region. Less than perfect complementarity refers to the situation in which some, but not all, nucleotide units of two strands or two regions can hydrogen bond with each other and can be expressed as a percentage.

A “target site” or “target sequence” is the nucleic acid sequence recognized (i.e., sufficiently complementary for hybridization) by an antisense oligonucleotide or inhibitory RNA molecule.

“Administering” a nucleic acid, such as a microRNA, siRNA, piRNA, snRNA, antisense nucleic acid, or lncRNA to a cell comprises introducing the nucleic acid into a cell by any means by which a nucleic acid can be transported across a cell membrane, including transducing, transfecting, electroporating, translocating, fusing, phagocytosing, shooting or ballistic methods, and so forth.

The term “transfection” is used to refer to the uptake of foreign DNA or RNA by a cell. A cell has been “transfected” when exogenous DNA or RNA has been introduced inside the cell membrane. The term refers to both stable and transient uptake of the genetic material, and includes uptake, for example, of microRNA, siRNA, piRNA, lncRNA, or antisense nucleic acids.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

By a “HOTAIR inhibitor”, a term that includes antagonists of HOTAIR, is meant any molecule (e.g., microRNA, siRNA, piRNA, snRNA, antisense nucleic acid, ribozyme, or small molecule inhibitor) that inhibits, suppresses or causes the cessation of at least HOTAIR-mediated biological activity, for example by interfering with transcription of HOTAIR or interfering with the interaction of HOTAIR to its target, such as a Polycomb-group protein Polycomb Repressive Complex 2 (PRC2).

An “effective amount” of a HOTAIR inhibitor or antagonist is an amount sufficient to effect beneficial or desired results, such as an amount that inhibits the activity of the lncRNA HOTAIR. An effective amount can be administered in one or more administrations, applications, or dosages.

By “anti-angiogenesis activity” is intended a reduction in the rate of branching, and hence a decline in angiogenesis. Such activity can be assessed using animal models.

By “therapeutically effective dose or amount” of a HOTAIR inhibitor is intended an amount that, when administered as described herein, brings about a positive therapeutic response, such as anti-angiogenesis activity. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

“Substantially purified” generally refers to isolation of a substance (compound, polynucleotide, protein, polypeptide, polypeptide composition) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

“Homology” refers to the percent identity between two polynucleotide or two polypeptide moieties. Two nucleic acid, or two polypeptide sequences are “substantially homologous” to each other when the sequences exhibit at least about 50% sequence identity, preferably at least about 75% sequence identity, more preferably at least about 80%-85% sequence identity, more preferably at least about 90% sequence identity, and most preferably at least about 95%-98% sequence identity over a defined length of the molecules. As used herein, substantially homologous also refers to sequences showing complete identity to the specified sequence.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353 358, National biomedical Research Foundation, Washington, D.C., which adapts the local homology algorithm of Smith and Waterman Advances in Appl. Math. 2:482 489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Other suitable programs for calculating the percent identity or similarity between sequences are generally known in the art, for example, another alignment program is BLAST, used with default parameters. Details of these programs are readily available.

Alternatively, homology can be determined by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells”, “host cells,” “cells”, “cell lines,” “cell cultures”, and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

“Operably linked” refers to an arrangement of elements wherein the components so described are configured so as to perform their usual function. Thus, a given promoter operably linked to a coding sequence is capable of effecting the expression of the coding sequence when the proper enzymes are present. Expression is meant to include the transcription of any one or more of transcription of a microRNA, siRNA, piRNA, snRNA, lncRNA, antisense nucleic acid, or mRNA from a DNA or RNA template and can further include translation of a protein from an mRNA template. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Purified polynucleotide” refers to a polynucleotide of interest or fragment thereof which is essentially free, e.g., contains less than about 50%, preferably less than about 70%, and more preferably less than about at least 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

The terms “variant” refers to biologically active derivatives of the reference molecule that retain desired activity, such as RNA interference (RNAi), lncRNA inhibition, or transcription factor inhibition. In general, the term “variant” refers to molecules (e.g., lncRNAs, miRNAs, siRNAs, piRNAs, snRNAs, antisense nucleic acids, or other inhibitors of lncRNAs) having a native sequence and structure with one or more additions, substitutions (generally conservative in nature) and/or deletions, relative to the native molecule, so long as the modifications do not destroy biological activity and which are “substantially homologous” to the reference molecule. In general, the sequences of such variants will have a high degree of sequence homology to the reference sequence, e.g., sequence homology of more than 50%, generally more than 60%-70%, even more particularly 80%-85% or more, such as at least 90%-95% or more, when the two sequences are aligned.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

The term “derived from” is used herein to identify the original source of a molecule but is not meant to limit the method by which the molecule is made which can be, for example, by chemical synthesis or recombinant means.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

A “biomarker” in the context of the present invention refers to an lncRNA which is differentially expressed in a biological sample (e.g. a tissue undergoing angiogenesis) as compared to a control sample (e.g., a comparable sample taken from a person with a negative diagnosis, a normal or healthy subject, or normal, untreated tissue or cells). The biomarker can be a lncRNA that can be detected and/or quantified. Biomarkers include, but are not limited to HOTAIR, MALAT1, H19, WISPER, ZFAS1, HULC, MIAT, ANRIL, MEG3.

MALAT1: metastasis-associated lung adenocarcinoma transcript 1 (MALAT1) is a highly conserved intergenic lncRNA that has implications in various cancers (35,36), neurological disorders (37), and cardiovascular disease (38).

H19: a conserved and maternally imprinted lncRNA, is one of the earliest identified lncRNAs (39).

WISPER (Wisp2 super-enhancer-associated RNA) is a novel and recently identified lncRNA that plays a prominent role in regulating cardiac fibrosis after injury (40).

ZFAS1: Highly expressed in the heart, the lncRNA ZFAS1 (ZNFX Antisense RNA 1) is a regulator of organ development, cancer growth and metastasis, apoptosis, and cell cycle regulation (41,42).

HULC: Highly upregulated in liver cancer (HULC) is a critical lncRNA that regulates angiogenesis, cell proliferation and migration, stem cell differentiation and lipid metabolism (43).

MIAT: Myocardial infarction-associated transcript (MIAT; also referred to as RNCR2, Gomafu, or AK028326) was originally identified in a case-control genome-wide association study, where 6 single nucleotide polymorphisms in the MIAT locus conferred susceptibility to myocardial infarction (MI) (44). Following this initial study, several experimental studies have emerged that shed light on the functional roles of MIAT in various biological and pathological processes, including schizophrenia (45), lung cancer (46), retinal and brain development (47, 48), and cataract formation (49).

ANRIL: Consisting of 19 exons and spanning nearly 126 kilobases (kb) (50), the antisense RNA to INK4 locus (ANRIL; also known as CDKN2B-AS1) gene gives rise to a 3.8-kb lncRNA that is prominently deregulated in cardiovascular disease (51) and several cancers (52).

MEG3: The maternally expressed gene 3 (MEG3) is a lncRNA gene that belongs to the DLK1—MEG3 imprinting locus and exerts critical developmental properties (53). Several lines of evidence also suggest that the inactivation of this gene and the subsequent loss of the MEG3 lncRNA are frequently documented in numerous cancers, suggesting important tumour-suppressive properties of this gene (54). In diabetic environments, the presence of MEG3 in the vitreous humor has not been reported yet.

The phrase “differentially expressed” refers to differences in the quantity and/or the frequency of a biomarker present in a sample taken from an animal model or human patients having, for example, DR or undergoing DR treatment as compared to a control subject. For example, a biomarker can be a lncRNA which is present at an elevated level or at a decreased level in samples of patients with DR or undergoing DR treatment compared to samples of control subjects. Alternatively, a biomarker can be a lncRNA which is detected at a higher frequency or at a lower frequency in samples of animal models or patients with DR or undergoing DR therapy compared to samples of control subjects or control tissues. A biomarker can be differentially present in terms of quantity, frequency or both.

A lncRNA is differentially expressed between two samples if the amount of the lncRNA in one sample is statistically significantly different from the amount of the lncRNA in the other sample.

Alternatively or additionally, a lncRNA is differentially expressed in two sets of samples if the frequency of detecting the lncRNA in samples is statistically significantly higher or lower than in the control samples.

The terms “subject,” “individual,” and “patient,” are used interchangeably herein and refer to any mammalian subject for whom diagnosis, prognosis, treatment, or therapy is desired, particularly humans. Other subjects may include cattle, dogs, cats, guinea pigs, rabbits, rats, mice, horses, and so on. In some cases, the methods of the invention find use in experimental animals, in veterinary application, and in the development of animal models for disease, including, but not limited to, rodents including mice, rats, and hamsters; primates, and transgenic animals.

As used herein, a “biological sample” refers to a sample of tissue or fluid isolated from a subject, including but not limited to, for example, vitreous humor (VH), tears, urine, blood, plasma, serum, fecal matter, bone marrow, bile, spinal fluid, lymph fluid, extracellular vesicles (e.g., exosomes), samples of the skin, external secretions of the skin, respiratory, intestinal, and genitourinary tracts, saliva, milk, blood cells, organs, biopsies, and also samples containing cells or tissues derived from the subject and grown in culture, and in vitro cell culture constituents, including but not limited to, conditioned media resulting from the growth of cells and tissues in culture, recombinant cells, stem cells, and cell components.

The terms “quantity,” “amount,” and “level” are used interchangeably herein and may refer to an absolute quantification of a molecule or an analyte in a sample, or to a relative quantification of a molecule or analyte in a sample, i.e., relative to another value such as relative to a reference value as taught herein, or to a range of values for the biomarker. These values or ranges can be obtained from a single patient or from a group of patients.

A “test amount” of a biomarker refers to an amount of a biomarker present in a sample being tested. A test amount can be either an absolute amount (e.g., μg/ml) or a relative amount (e.g., relative intensity of signals).

A “diagnostic amount” of a biomarker refers to an amount of a biomarker in a subject's sample that is consistent with a diagnosis of DR or angiogenesis. A diagnostic amount can be either an absolute amount (e.g., μg/ml) or a relative amount (e.g., relative intensity of signals).

A “control amount” or “control reference value” of a marker can be any amount or a range of amount which is to be compared against a test amount of a biomarker. For example, a control amount of a biomarker can be the amount of a biomarker in a person without DR, or normal tissue or cells, or untreated tissue or cells. A control amount can be either in absolute amount (e.g., μg/ml) or a relative amount (e.g., relative intensity of signals).

The term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)2 and F(ab) fragments; Fv molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

“Immunoassay” is an assay that uses an antibody to specifically bind an antigen (e.g., a biomarker). The immunoassay is characterized by the use of specific binding properties of a particular antibody to isolate, target, and/or quantify the antigen. An immunoassay for a biomarker may utilize one antibody or several antibodies. Immunoassay protocols may be based, for example, upon competition, direct reaction, or sandwich type assays using, for example, labeled antibody. The labels may be, for example, fluorescent, chemiluminescent, electrochemical, or radioactive.

The phrase “specifically (or selectively) binds” to an antibody or “specifically (or selectively) immunoreactive with,” when referring to a biomarker, refers to a binding reaction that is determinative of the presence of the biomarker in a heterogeneous population of proteins, nucleic acids, and other biologics. Specific binding to an antibody under such conditions may require an antibody that is selected for its specificity for a particular lncRNA. For example, polyclonal antibodies raised to a biomarker from specific species such as rat, mouse, or human can be selected to obtain only those polyclonal antibodies that are specifically immunoreactive with the biomarker and not with other nucleic acids, except for polymorphic variants and alleles of the biomarker. This selection may be achieved by subtracting out antibodies that cross-react with biomarker molecules from other species. A variety of immunoassay formats may be used to select antibodies specifically immunoreactive with a particular biomarker. For example, solid-phase ELISA immunoassays are routinely used to select antibodies specifically immunoreactive with an antigen (see, e.g., Harlow & Lane. Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Capture reagent” refers to a molecule or group of molecules that specifically bind to a specific target molecule or group of target molecules. For example, a capture reagent can comprise two or more antibodies with each antibody having specificity for a separate target molecule. Capture reagents can be any combination of organic or inorganic chemicals, or biomolecules, and all fragments, analogs, homologs, conjugates, and derivatives thereof that can specifically bind a target molecule.

The capture reagent can comprise a single molecule that can form a complex with multiple targets, for example, a multimeric fusion protein with multiple binding sites for different targets. The capture reagent can comprise multiple molecules each having specificity for a different target, thereby resulting in multiple capture reagent-target complexes. In certain embodiments, the capture reagent is comprised of proteins, such as antibodies.

The capture reagent can be directly labeled with a detectable moiety. For example, an anti-biomarker antibody can be directly conjugated to a detectable moiety and used in the inventive methods, devices, and kits. In the alternative, detection of the capture reagent-biomarker complex can be by a secondary reagent that specifically binds to the biomarker or the capture reagent-biomarker complex. The secondary reagent can be any biomolecule, and is preferably an antibody. The secondary reagent is labeled with a detectable moiety. In some embodiments, the capture reagent or secondary reagent is coupled to biotin, and contacted with avidin or streptavidin having a detectable moiety tag.

“Detectable moieties” or “detectable labels” contemplated for use in the invention include, but are not limited to, radioisotopes, fluorescent dyes such as RNA Mango technology based on the specific binding of the RNA Mango Aptamer and a Thizole Orange (TO) bi-functional dye, fluorescein, phycoerythrin, Cy-3, Cy-5, allophycoyanin, DAPI, Texas Red, rhodamine, Oregon green, Lucifer yellow, and the like, green fluorescent protein (GFP), red fluorescent protein (DsRed), Thizole Orange bi-functional dye, Cyan Fluorescent Protein (CFP), Yellow Fluorescent Protein (YFP), Cerianthus Orange Fluorescent Protein (cOFP), alkaline phosphatase (AP), beta-lactamase, chloramphenicol acetyltransferase (CAT), adenosine deaminase (ADA), aminoglycoside phosphotransferase (neor, G418r) dihydrofolate reductase (DHFR), hygromycin-B-phosphotransferase (HPH), thymidine kinase (TK), lacZ (encoding alpha-galactosidase), and xanthine guanine phosphoribosyltransferase (XGPRT), Beta-Glucuronidase (gus), Placental Alkaline Phosphatase (PLAP), Secreted Embryonic Alkaline Phosphatase (SEAP), or Firefly or Bacterial Luciferase (LUC). Enzyme tags are used with their cognate substrate. The terms also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), and glass microparticles with digital holographic code images (see e.g., CyVera microbeads produced by Illumina (San Diego, Calif.). As with many of the standard procedures associated with the practice of the invention, skilled artisans will be aware of additional labels that can be used.

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

### MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on lncRNAs that play roles in regulation of high glucose induced angiogenesis and diabetes-related processes. Such lncRNAs can be used as biomarkers to monitor chronic diabetic complications, such as DR.

A. Biomarkers and Methods of Diagnosis

Biomarkers that can be used in the practice of the invention include lncRNAs such as, but not limited to HOTAIR, MALAT1, H19, WISPER, ZFAS1, HULC, MIAT, ANRIL, MEG3.

Accordingly, in one aspect, the invention provides a method for diagnosing patients at risk of developing or having an increased likelihood of progressing to end-organ damage due to diabetes, comprising measuring the levels of one or more of HOTAIR, MALAT1, H19, WISPER, ZFAS1, HULC, MIAT, ANRIL, and/or MEG3 in a biological sample derived from a subject suspected of having diabetes, and analyzing the levels of the biomarkers and comparing with respective control reference value ranges for the biomarkers, wherein differential or altered expression of the one or more of the biomarkers in the biological sample compared to the one or more biomarkers in a control sample indicates that the subject is at risk of developing end-organ damage due to diabetes. The biomarkers can be used alone or in combination with relevant clinical parameters in prognosis, diagnosis, or monitoring treatment of diabetes. In another embodiment, HOTAIR is used alone or in combination with one or more additional biomarkers or clinical parameters in diagnosing patients at risk of developing end-organ damage due to diabetes. Following a positive diagnosis of a patient at risk of developing end-organ damage due to diabetes, the method may further comprise treating the patient from developing said end-organ damage due to diabetes.

In another embodiment, the present invention is a method of diagnosing diabetic retinopathy (DR) in a subject, the method comprising: a) measuring the amount of a long non-coding RNA (lncRNA) in a biological sample derived from the subject; and b) comparing the amount of the lncRNA with a control reference value, and when the amount of the lncRNA is altered (i.e., increased or decreased) relative to the control reference value, diagnosing the subject as having DR, wherein the lncRNA is one or more of HOTAIR, H19, WISPER, ZFAS1, HULC, ANRIL, MALAT1, MIAT, and MEG3. Following a positive diagnosis of a patient as having DR, the method may further comprise treating the patient for DR.

When analyzing the levels of biomarkers in a biological sample, the reference value ranges used for comparison can represent the level of one or more biomarkers found in one or more samples of one or more subjects without diabetes (i.e., normal or negative control samples). Alternatively, the reference values can represent the level of one or more biomarkers found in one or more samples of one or more subjects with diabetes (i.e., positive control samples). More specifically, the reference value ranges can represent the level of one or more biomarkers at particular stages of disease to facilitate a determination of the stage of disease progression in an individual.

In another embodiment, the invention includes a method for monitoring the efficacy of a therapy for treating a disease or condition in a subject, the method comprising: analyzing the level of each of one or more biomarkers in samples derived from the subject before and after the subject undergoes said therapy, in conjunction with respective reference value ranges for said one or more biomarkers, wherein the one or more biomarkers comprises one or more lncRNAs selected from HOTAIR, MALAT1, H19, WISPER, ZFAS1, HULC, MIAT, ANRIL, and/or MEG3. In embodiments, the disease or condition is a diabetes-induced disease or condition. In embodiments, the disease or condition is one or more of: non-proliferative and proliferative diabetic retinopathy, diabetic nephropathy, diabetic cardiomyopathy, diabetic neuropathy, proliferative vitreoretinopathies, neovascular glaucoma, ischemic retinopathy, retinopathy secondary to retinal vein occlusion, as well as age-related macular degeneration, keloid formation, and wound healing.

In another embodiment, the invention includes a method for evaluating the effect of an agent for treating a disease or condition in a subject, the method comprising: analyzing the level of each of one or more biomarkers in samples derived from the subject before and after the subject is treated with said agent, in conjunction with respective reference value ranges for said one or more biomarkers, wherein one or more biomarkers comprises one or more lncRNAs selected from HOTAIR, MALAT1, H19, WISPER, ZFAS1, HULC, MIAT, ANRIL, and/or MEG3. In embodiments, the disease or condition is a diabetes-induced disease or condition. In embodiments, the disease or condition is one or more of: non-proliferative and proliferative diabetic retinopathy, diabetic nephropathy, diabetic cardiomyopathy, diabetic neuropathy, proliferative vitreoretinopathies, neovascular glaucoma, ischemic retinopathy, retinopathy secondary to retinal vein occlusion, as well as age-related macular degeneration, keloid formation, and wound healing.

In certain embodiments, the invention includes a biomarker panel comprising a plurality of lncRNAs selected from the group consisting of HOTAIR, MALAT1, H19, WISPER, ZFAS1, HULC, MIAT, ANRIL, and/or MEG3.

The methods described herein for prognosis or diagnosis of a disease or condition, may be used in individuals who have not yet been diagnosed (for example, preventative screening), or who have been diagnosed, or who are suspected of having diabetes, or who are at risk of developing diabetes (e.g., have a genetic predisposition or presence of one or more developmental, environmental, or behavioral risk factors). The methods may also be used to detect various stages of progression or severity of disease. The methods may also be used to detect the response of disease to prophylactic or therapeutic treatments or other interventions. The methods can furthermore be used to help the medical practitioner in determining prognosis (e.g., worsening, status-quo, partial recovery, or complete recovery) of the patient, and the appropriate course of action, resulting in either further treatment or observation, or in discharge of the patient from the medical care center.

B. Detecting and Measuring Levels of Biomarkers

It is understood that the expression level of the biomarkers of the present invention in a sample can be determined by any suitable method known in the art. Measurement of the level of a biomarker can be direct or indirect. For example, the abundance levels of lncRNAs can be directly quantitated. Alternatively, the amount of a biomarker can be determined indirectly by measuring abundance levels of cDNAs, amplified RNAs or DNAs, or by measuring quantities or activities of RNAs, or other molecules that are indicative of the expression level of the biomarker.

LncRNAs can be detected and quantitated by a variety of methods including, but not limited to, microarray analysis, next-generation sequencing (such as RNA sequencing), polymerase chain reaction (PCR), reverse transcriptase polymerase chain reaction (RT-PCR), Northern blot, serial analysis of gene expression (SAGE), immunoassay, and mass spectrometry. See, e.g., Draghici Data Analysis Tools for DNA Microarrays, Chapman and Hall/CRC, 2003; Simon et al. Design and Analysis of DNA Microarray Investigations, Springer, 2004; Real-Time PCR: Current Technology and Applications, Logan, Edwards, and Saunders eds., Caister Academic Press, 2009; Bustin A-Z of Quantitative PCR (IUL Biotechnology, No. 5), International University Line, 2004; Velculescu et al. (1995) Science 270: 484-487; Matsumura et al. (2005) Cell. Microbiol. 7: 11-18; Serial Analysis of Gene Expression (SAGE): Methods and Protocols (Methods in Molecular Biology), Humana Press, 2008, Hoffmann and Stroobant Mass Spectrometry: Principles and Applications, Third Edition, Wiley, 2007; herein incorporated by reference in their entireties.

In one embodiment, microarrays are used to measure the levels of biomarkers. An advantage of microarray analysis is that the expression of each of the biomarkers can be measured simultaneously, and microarrays can be specifically designed to provide a diagnostic expression profile for a particular disease or condition.

Microarrays are prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

Probes used in the methods of the invention are preferably immobilized to a solid support which may be either porous or non-porous. For example, the probes may be polynucleotide sequences which are attached to a nitrocellulose or nylon membrane or filter covalently at either the 3′ or the 5′ end of the polynucleotide. Such hybridization probes are well known in the art (see, e.g., Sambrook, et al., Molecular Cloning: A Laboratory Manual (3rd Edition, 2001). Alternatively, the solid support or surface may be a glass or plastic surface. In one embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel.

In one embodiment, the microarray comprises a support or surface with an ordered array of binding (e.g., hybridization) sites or “probes” each representing one of the biomarkers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). Each probe is preferably covalently attached to the solid support at a single site.

Microarrays can be made in a number of ways, of which several are described below. However they are produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. Microarrays are generally small, e.g., between 1 cm² and 25 cm²; however, larger arrays may also be used, e.g., in screening arrays. Preferably, a given binding site or unique set of binding sites in the microarray will specifically bind (e.g., hybridize) to the product of a single gene in a cell (e.g., to a specific mRNA, lncRNA, or to a specific cDNA derived therefrom). However, in general, other related or similar sequences will cross hybridize to a given binding site.

As noted above, the “probe” to which a particular polynucleotide molecule specifically hybridizes contains a complementary polynucleotide sequence. The probes of the microarray typically consist of nucleotide sequences of no more than 1,000 nucleotides. In some embodiments, the probes of the array consist of nucleotide sequences of 10 to 1,000 nucleotides. In one embodiment, the nucleotide sequences of the probes are in the range of 10-200 nucleotides in length and are genomic sequences of one species of organism, such that a plurality of different probes is present, with sequences complementary and thus capable of hybridizing to the genome of such a species of organism, sequentially tiled across all or a portion of the genome. In other embodiments, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, or are 60 nucleotides in length.

The probes may comprise DNA or DNA “mimics” (e.g., derivatives and analogues) corresponding to a portion of an organism's genome. In another embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone (e.g., phosphorothioates).

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically each probe on the microarray will be between 10 bases and 50,000 bases, usually between 300 bases and 1,000 bases in length. PCR methods are well known in the art, and are described, for example, in Innis et al., eds., PCR Protocols: A Guide To Methods And Applications, Academic Press Inc., San Diego, Calif. (1990); herein incorporated by reference in its entirety. It will be apparent to one skilled in the art that controlled robotic systems are useful for isolating and amplifying nucleic acids.

An alternative, preferred means for generating polynucleotide probes is by synthesis of synthetic polynucleotides or oligonucleotides, e.g., using N-phosphonate or phosphoramidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., Nature 363:566-568 (1993); U.S. Pat. No. 5,539,083).

Probes are preferably selected using an algorithm that takes into account binding energies, base composition, sequence complexity, cross-hybridization binding energies, and secondary structure. See Friend et al., International Patent Publication WO 01/05935, published Jan. 25, 2001; Hughes et al., Nat. Biotech. 19:342-7 (2001).

A skilled artisan will also appreciate that positive control probes, e.g., probes known to be complementary and hybridizable to sequences in the target polynucleotide molecules, and negative control probes, e.g., probes known to not be complementary and hybridizable to sequences in the target polynucleotide molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

The probes are attached to a solid support or surface, which may be made, e.g., from glass, plastic (e.g., polypropylene, nylon), polyacrylamide, nitrocellulose, gel, or other porous or nonporous material. One method for attaching nucleic acids to a surface is by printing on glass plates, as is described generally by Schena et al, Science 270:467-470 (1995). This method is especially useful for preparing microarrays of cDNA (See also, DeRisi et al, Nature Genetics 14:457-460 (1996); Shalon et al., Genome Res. 6:639-645 (1996); and Schena et al., Proc. Natl. Acad. Sci. U.S.A. 93:10539-11286 (1995); herein incorporated by reference in their entireties).

A second method for making microarrays produces high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270; herein incorporated by reference in their entireties) or other methods for rapid synthesis and deposition of defined oligonucleotides (Blanchard et al., Biosensors & Bioelectronics 11:687-690; herein incorporated by reference in its entirety). When these methods are used, oligonucleotides (e.g., 60-mers) of known sequence are synthesized directly on a surface such as a derivatized glass slide. Usually, the array produced is redundant, with several oligonucleotide molecules per RNA.

Other methods for making microarrays, e.g., by masking (Maskos and Southern, 1992, Nuc. Acids. Res. 20:1679-1684; herein incorporated by reference in its entirety), may also be used. In principle, any type of array, for example, dot blots on a nylon hybridization membrane (see Sambrook, et al., Molecular Cloning: A Laboratory Manual, 3rd Edition, 2001) could be used. However, as will be recognized by those skilled in the art, very small arrays will frequently be preferred because hybridization volumes will be smaller.

Microarrays can also be manufactured by means of an ink jet printing device for oligonucleotide synthesis, e.g., using the methods and systems described by Blanchard in U.S. Pat. No. 6,028,189; Blanchard et al., 1996, Biosensors and Bioelectronics 11:687-690; Blanchard, 1998, in Synthetic DNA Arrays in Genetic Engineering, Vol. 20, J. K. Setlow, Ed., Plenum Press, New York at pages 111-123; herein incorporated by reference in their entireties. Specifically, the oligonucleotide probes in such microarrays are synthesized in arrays, e.g., on a glass slide, by serially depositing individual nucleotide bases in “microdroplets” of a high surface tension solvent such as propylene carbonate. The microdroplets have small volumes (e.g., 100 μL or less, more preferably 50 μL or less) and are separated from each other on the microarray (e.g., by hydrophobic domains) to form circular surface tension wells which define the locations of the array elements (i.e., the different probes). Microarrays manufactured by this ink jet method are typically of high density, preferably having a density of at least about 2,500 different probes per 1 cm2. The polynucleotide probes are attached to the support covalently at either the 3′ or the 5′ end of the polynucleotide.

Biomarker polynucleotides which may be measured by microarray analysis can be expressed lncRNAs or a nucleic acid derived therefrom (e.g., cDNA or amplified RNA derived from cDNA that incorporates an RNA polymerase promoter), including naturally occurring nucleic acid molecules, as well as synthetic nucleic acid molecules. In one embodiment, the target polynucleotide molecules comprise RNA, including, but by no means limited to, total cellular RNA, lncRNA, poly(A)+ messenger RNA (mRNA) or a fraction thereof, cytoplasmic mRNA, or RNA transcribed from cDNA (i.e., cRNA; see, e.g., Linsley & Schelter, U.S. patent application Ser. No. 09/411,074, filed Oct. 4, 1999, or U.S. Pat. Nos. 5,545,522, 5,891,636, or 5,716,785). Methods for preparing total and poly(A)+ RNA are well known in the art, and are described generally, e.g., in Sambrook, et al., Molecular Cloning: A Laboratory Manual (3rd Edition, 2001). RNA can be extracted from a cell of interest using guanidinium thiocyanate lysis followed by CsCl centrifugation (Chirgwin et al., 1979, Biochemistry 18:5294-5299), a silica gel-based column (e.g., RNeasy (Qiagen, Valencia, Calif.) or StrataPrep (Stratagene, La Jolla, Calif.)), or using phenol and chloroform, as described in Ausubel et al., eds., 1989, Current Protocols In Molecular Biology, Vol. III, Green Publishing Associates, Inc., John Wiley & Sons, Inc., New York, at pp. 13.12.1-13.12.5). Poly(A)+ RNA can be selected, e.g., by selection with oligo-dT cellulose or, alternatively, by oligo-dT primed reverse transcription of total cellular RNA. RNA can be fragmented by methods known in the art, e.g., by incubation with ZnCl2, to generate fragments of RNA.

In one embodiment, total RNA, lncRNAs, or nucleic acids derived therefrom, are isolated from a sample taken from a patient undergoing medical treatment for DR. Biomarker lncRNAs that are poorly expressed in particular cells may be enriched using normalization techniques (Bonaldo et al., 1996, Genome Res. 6:791-806).

As described above, the biomarker polynucleotides can be detectably labeled at one or more nucleotides. Any method known in the art may be used to label the target polynucleotides. Preferably, this labeling incorporates the label uniformly along the length of the RNA, and more preferably, the labeling is carried out at a high degree of efficiency. For example, polynucleotides can be labeled by oligo-dT primed reverse transcription. Random primers (e.g., 9-mers) can be used in reverse transcription to uniformly incorporate labeled nucleotides over the full length of the polynucleotides. Alternatively, random primers may be used in conjunction with PCR methods or T7 promoter-based in vitro transcription methods in order to amplify polynucleotides.

The detectable label may be a luminescent label. For example, fluorescent labels, bioluminescent labels, chemiluminescent labels, and colorimetric labels may be used in the practice of the invention. Fluorescent labels that can be used include, but are not limited to, fluorescein, a phosphor, a rhodamine, or a polymethine dye derivative. Additionally, commercially available fluorescent labels including, but not limited to, fluorescent phosphoramidites such as FluorePrime (Amersham Pharmacia, Piscataway, N.J.), Fluoredite (Miilipore, Bedford, Mass.), FAM (ABI, Foster City, Calif.), and Cy3 or Cy5 (Amersham Pharmacia, Piscataway, N.J.) can be used. Alternatively, the detectable label can be a radiolabeled nucleotide.

In one embodiment, biomarker polynucleotide molecules from a patient sample are labeled differentially from the corresponding polynucleotide molecules of a reference sample. The reference can comprise lncRNAs from a normal biological sample (i.e., control sample, e.g., biopsy from a subject not having diabetes, or untreated cells or tissue) or from a reference biological sample, (e.g., sample from a subject having diabetes, sample of cells or tissue at different stages of differentiation or treatment).

Nucleic acid hybridization and wash conditions are chosen so that the target polynucleotide molecules specifically bind or specifically hybridize to the complementary polynucleotide sequences of the array, preferably to a specific array site, wherein its complementary DNA is located. Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self-complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook, et al., Molecular Cloning: A Laboratory Manual (3rd Edition, 2001), and in Ausubel et al., Current Protocols In Molecular Biology, vol. 2, Current Protocols Publishing, New York (1994). Typical hybridization conditions for the cDNA microarrays of Schena et al. are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Schena et al., Proc. Natl. Acad. Sci. U.S.A. 93:10614 (1993)). Useful hybridization conditions are also provided in, e.g., Tijessen, 1993, Hybridization with Nucleic Acid Probes, Elsevier Science Publishers B. V.; and Kricka, 1992, Nonisotopic Dna Probe Techniques, Academic Press, San Diego, Calif. Particularly preferred hybridization conditions include hybridization at a temperature at or near the mean melting temperature of the probes (e.g., within 51° C., more preferably within 21° C.) in 1 M NaCl, 50 mM MES buffer (pH 6.5), 0.5% sodium sarcosine and 30% formamide.

When fluorescently labeled gene products are used, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously (see Shalon et al., 1996, “A DNA microarray system for analyzing complex DNA samples using two-color fluorescent probe hybridization,” Genome Research 6:639-645, which is incorporated by reference in its entirety for all purposes). Arrays can be scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., Genome Res. 6:639-645 (1996), and in other references cited herein. Alternatively, the fiber-optic bundle described by Ferguson et al., Nature Biotech. 14:1681-1684 (1996), may be used to monitor mRNA abundance levels at a large number of sites simultaneously.

In one embodiment, the invention includes a microarray comprising a plurality of probes that hybridize to one or more lncRNAs selected from HOTAIR, MALAT1, H19, WISPER, ZFAS1, HULC, MIAT, ANRIL, and/or MEG3.

Polynucleotides can also be analyzed by other methods including, but not limited to, northern blotting, nuclease protection assays, RNA fingerprinting, polymerase chain reaction, ligase chain reaction, Qbeta replicase, isothermal amplification method, strand displacement amplification, transcription based amplification systems, nuclease protection (Si nuclease or RNAse protection assays), SAGE as well as methods disclosed in International Publication Nos. WO 88/10315 and WO 89/06700, and International Applications Nos. PCT/US87/00880 and PCT/US89/01025; herein incorporated by reference in their entireties.

A standard Northern blot assay can be used to ascertain an RNA transcript size, identify alternatively spliced RNA transcripts, and the relative amounts of mRNA or lncRNA in a sample, in accordance with conventional Northern hybridization techniques known to those persons of ordinary skill in the art. In Northern blots, RNA samples are first separated by size by electrophoresis in an agarose gel under denaturing conditions. The RNA is then transferred to a membrane, cross-linked, and hybridized with a labeled probe. Nonisotopic or high specific activity radiolabeled probes can be used, including random-primed, nick-translated, or PCR-generated DNA probes, in vitro transcribed RNA probes, and oligonucleotides. Additionally, sequences with only partial homology (e.g., cDNA from a different species or genomic DNA fragments that might contain an exon) may be used as probes. The labeled probe, e.g., a radiolabelled cDNA, either containing the full-length, single stranded DNA or a fragment of that DNA sequence may be at least 20, at least 30, at least 50, or at least 100 consecutive nucleotides in length. The probe can be labeled by any of the many different methods known to those skilled in this art. The labels most commonly employed for these studies are radioactive elements, enzymes, chemicals that fluoresce when exposed to ultraviolet light, and others. A number of fluorescent materials are known and can be utilized as labels. These include, but are not limited to, fluorescein, rhodamine, auramine, Texas Red, AMCA blue and Lucifer Yellow. A particular detecting material is anti-rabbit antibody prepared in goats and conjugated with fluorescein through an isothiocyanate. Proteins can also be labeled with a radioactive element or with an enzyme. The radioactive label can be detected by any of the currently available counting procedures. Isotopes that can be used include, but are not limited to, 3H, 14C, 32P, 35S, 36Cl, 35Cr, 57Co, 58Co, 59Fe, 90Y, 125I, 131I, and 186Re. Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Any enzymes known to one of skill in the art can be utilized. Examples of such enzymes include, but are not limited to, peroxidase, beta-D-galactosidase, urease, glucose oxidase plus peroxidase and alkaline phosphatase. U.S. Pat. Nos. 3,654,090, 3,850,752, and 4,016,043 are referred to by way of example for their invention of alternate labeling material and methods.

Nuclease protection assays (including both ribonuclease protection assays and Si nuclease assays) can be used to detect and quantitate specific mRNAs and lncRNAs. In nuclease protection assays, an antisense probe (labeled with, e.g., radiolabeled or nonisotopic) hybridizes in solution to an RNA sample. Following hybridization, single-stranded, unhybridized probe and RNA are degraded by nucleases. An acrylamide gel is used to separate the remaining protected fragments. Typically, solution hybridization is more efficient than membrane-based hybridization, and it can accommodate up to 100 μg of sample RNA, compared with the 20-30 μg maximum of blot hybridizations.

The ribonuclease protection assay, which is the most common type of nuclease protection assay, requires the use of RNA probes. Oligonucleotides and other single-stranded DNA probes can only be used in assays containing Si nuclease. The single-stranded, antisense probe must typically be completely homologous to target RNA to prevent cleavage of the probe:target hybrid by nuclease.

Serial Analysis Gene Expression (SAGE), can also be used to determine RNA (e.g., lncRNA) abundances in a cell sample. See, e.g., Velculescu et al., 1995, Science 270:484-7; Carulli, et al., 1998, Journal of Cellular Biochemistry Supplements 30/31:286-96; herein incorporated by reference in their entireties. SAGE analysis does not require a special device for detection, and is one of the preferable analytical methods for simultaneously detecting the expression of a large number of transcription products. First, RNA is extracted from cells. Next, the RNA is converted into cDNA using a biotinylated oligo (dT) primer, and treated with a four-base recognizing restriction enzyme (Anchoring Enzyme: AE) resulting in AE-treated fragments containing a biotin group at their 3′ terminus. Next, the AE-treated fragments are incubated with streptoavidin for binding. The bound cDNA is divided into two fractions, and each fraction is then linked to a different double-stranded oligonucleotide adapter (linker) A or B. These linkers are composed of: (1) a protruding single strand portion having a sequence complementary to the sequence of the protruding portion formed by the action of the anchoring enzyme, (2) a 5′ nucleotide recognizing sequence of the IIS-type restriction enzyme (cleaves at a predetermined location no more than 20 bp away from the recognition site) serving as a tagging enzyme (TE), and (3) an additional sequence of sufficient length for constructing a PCR-specific primer. The linker-linked cDNA is cleaved using the tagging enzyme, and only the linker-linked cDNA sequence portion remains, which is present in the form of a short-strand sequence tag. Next, pools of short-strand sequence tags from the two different types of linkers are linked to each other, followed by PCR amplification using primers specific to linkers A and B. As a result, the amplification product is obtained as a mixture comprising myriad sequences of two adjacent sequence tags (ditags) bound to linkers A and B. The amplification product is treated with the anchoring enzyme, and the free ditag portions are linked into strands in a standard linkage reaction. The amplification product is then cloned. Determination of the clone's nucleotide sequence can be used to obtain a read-out of consecutive ditags of constant length. The presence of mRNA corresponding to each tag can then be identified from the nucleotide sequence of the clone and information on the sequence tags.

Quantitative reverse transcriptase PCR (qRT-PCR) can also be used to determine the expression profiles of biomarkers (see, e.g., U.S. Patent Application Publication No. 2005/0048542A1; herein incorporated by reference in its entirety). The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN RT-PCR can be performed using commercially available equipment, such as, for example, ABI PRISM 7700 sequence detection system. (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700 sequence detection system. The system consists of a thermocycler, laser, charge-coupled device (CCD), camera and computer. The system includes software for running the instrument and for analyzing the data. 5′-Nuclease assay data are initially expressed as Ct, or the threshold cycle. Fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and beta-actin (β-actin).

A more recent variation of the RT-PCR technique is the real time quantitative PCR, which measures PCR product accumulation through a dual-labeled fluorigenic probe (i.e., TAQMAN probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

Mass spectrometry, and particularly SELDI mass spectrometry, is a particularly useful method for detection of the biomarkers of this invention. Laser desorption time-of-flight mass spectrometer can be used in embodiments of the invention. In laser desorption mass spectrometry, a substrate or a probe comprising biomarkers is introduced into an inlet system. The biomarkers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Matrix-assisted laser desorption/ionization mass spectrometry (MALDI-MS) can also be used for detecting the biomarkers of this invention. MALDI-MS is a method of mass spectrometry that involves the use of an energy absorbing molecule, frequently called a matrix, for desorbing proteins intact from a probe surface. MALDI is described, for example, in U.S. Pat. No. 5,118,937 (Hillenkamp et al.) and U.S. Pat. No. 5,045,694 (Beavis and Chait). In MALDI-MS, the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry.

Surface-enhanced laser desorption/ionization mass spectrometry, or SELDI-MS represents an improvement over MALDI for the fractionation and detection of biomolecules, such as lncRNAs, in complex mixtures. SELDI is a method of mass spectrometry in which biomolecules, such as lncRNAs, are captured on the surface of a biochip using capture reagents that are bound there. Typically, non-bound molecules are washed from the probe surface before interrogation. SELDI is described, for example, in: U.S. Pat. No. 5,719,060 (“Method and Apparatus for Desorption and Ionization of Analytes,” Hutchens and Yip, Feb. 17, 1998,) U.S. Pat. No. 6,225,047 (“Use of Retentate Chromatography to Generate Difference Maps,” Hutchens and Yip, May 1, 2001) and Weinberger et al., “Time-of-flight mass spectrometry,” in Encyclopedia of Analytical Chemistry, R. A. Meyers, ed., pp 11915-11918 John Wiley & Sons Chichesher, 2000.

Biomarkers on the substrate surface can be desorbed and ionized using gas phase ion spectrometry. Any suitable gas phase ion spectrometer can be used as long as it allows biomarkers on the substrate to be resolved. Preferably, gas phase ion spectrometers allow quantitation of biomarkers. In one embodiment, a gas phase ion spectrometer is a mass spectrometer. In a typical mass spectrometer, a substrate or a probe comprising biomarkers on its surface is introduced into an inlet system of the mass spectrometer. The biomarkers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of biomarkers or other substances will typically involve detection of signal intensity. This, in turn, can reflect the quantity and character of biomarkers bound to the substrate. Any of the components of a mass spectrometer (e.g., a desorption source, a mass analyzer, a detector, etc.) can be combined with other suitable components described herein or others known in the art in embodiments of the invention.

Biomarkers can also be detected with assays based on the use of antibodies that specifically recognize the lncRNA biomarkers or polynucleotide or oligonucleotide fragments of the biomarkers. Such assays include, but are not limited to, immunohistochemistry (1HC), enzyme-linked immunosorbent assay (ELISA), radioimmunoassays (MA), “sandwich” immunoassays, fluorescent immunoassays, immunoprecipitation assays, the procedures of which are well known in the art (see, e.g., Ausubel et al, eds, 1994, Current Protocols in Molecular Biology, Vol. 1, John Wiley & Sons, Inc., New York, which is incorporated by reference herein in its entirety).

Antibodies that specifically bind to a biomarker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). A biomarker antigen can be used to immunize a mammal, such as a mouse, rat, rabbit, guinea pig, monkey, or human, to produce polyclonal antibodies. If desired, a biomarker antigen can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a biomarker antigen can be prepared using any technique which provides for the production of antibody molecules by continuous cell lines in culture. These techniques include, but are not limited to, the hybridoma technique, the human B cell hybridoma technique, and the EBV hybridoma technique (Kohler et al., Nature 256, 495-97, 1985; Kozbor et al., J. Immunol. Methods 81, 3142, 1985; Cote et al., Proc. Natl. Acad. Sci. 80, 2026-30, 1983; Cole et al., Mol. Cell. Biol. 62, 109-20, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 6851-55, 1984; Neuberger et al., Nature 312, 604-08, 1984; Takeda et al., Nature 314, 452-54, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions.

Alternatively, humanized antibodies can be produced using recombinant methods, as described below. Antibodies which specifically bind to a particular antigen can contain antigen binding sites which are either partially or fully humanized, as disclosed in U.S. Pat. No. 5,565,332. Human monoclonal antibodies can be prepared in vitro as described in Simmons et al., PLoS Medicine 4(5), 928-36, 2007.

Alternatively, techniques described for the production of single chain antibodies can be adapted using methods known in the art to produce single chain antibodies which specifically bind to a particular antigen. Antibodies with related specificity, but of distinct idiotypic composition, can be generated by chain shuffling from random combinatorial immunoglobulin libraries (Burton, Proc. Natl. Acad. Sci. 88, 11120-23, 1991).

Single-chain antibodies also can be constructed using a DNA amplification method, such as PCR, using hybridoma cDNA as a template (Thirion et al., Eur. J. Cancer Prey. 5, 507-11, 1996). Single-chain antibodies can be mono- or bispecific, and can be bivalent or tetravalent. Construction of tetravalent, bispecific single-chain antibodies is taught, for example, in Coloma & Morrison, Nat. Biotechnol. 15, 159-63, 1997. Construction of bivalent, bispecific single-chain antibodies is taught in Mallender & Voss, J. Biol. Chem. 269, 199-206, 1994.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., Int. J. Cancer 61, 497-501, 1995; Nicholls et al., J. Immunol. Meth. 165, 81-91, 1993).

Antibodies which specifically bind to a biomarker antigen also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833 3837, 1989; Winter et al., Nature 349, 293 299, 1991).

Chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies can be purified by methods well known in the art. For example, antibodies can be affinity purified by passage over a column to which the relevant antigen is bound. The bound antibodies can then be eluted from the column using a buffer with a high salt concentration.

Antibodies may be used in diagnostic assays to detect the presence or for quantification of the biomarkers in a biological sample. Such a diagnostic assay may comprise at least two steps; (i) contacting a biological sample with the antibody, wherein the sample is a tissue (e.g., human, animal, etc.), cell (e.g., stem cell), extracellular vesicles (exosomes), biological fluid (e.g., blood, urine, sputum, semen, amniotic fluid, saliva, etc.), biological extract (e.g., tissue or cellular homogenate, etc.), or a chromatography column, etc; and (ii) quantifying the antibody bound to the substrate. The method may additionally involve a preliminary step of attaching the antibody, either covalently, electrostatically, or reversibly, to a solid support, before subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp 147-158). The antibodies used in the diagnostic assays can be labeled with a detectable moiety. The detectable moiety should be capable of producing, either directly or indirectly, a detectable signal. For example, the detectable moiety may be a radioisotope, such as 2H, 14C, 32P, or 125I, a fluorescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); David et al., Biochem., 13:1014 (1974); Pain et al., J. Immunol. Methods, 40:219 (1981); and Nygren, J. Histochem. and Cytochem., 30:407 (1982).

Immunoassays can be used to determine the presence or absence of a biomarker in a sample as well as the quantity of a biomarker in a sample. First, a test amount of a biomarker in a sample can be detected using the immunoassay methods described above. If a biomarker is present in the sample, it will form an antibody-biomarker complex with an antibody that specifically binds the biomarker under suitable incubation conditions, as described above. The amount of an antibody-biomarker complex can be determined by comparing to a standard. A standard can be, e.g., a known compound or another lncRNA known to be present in a sample. As noted above, the test amount of a biomarker need not be measured in absolute units, as long as the unit of measurement can be compared to a control.

Kits

In yet another aspect, the invention provides kits for use in diagnosing a disease or condition. In embodiments, the disease or condition is a diabetes-induced disease or condition. In embodiments, the disease or condition is one or more of: non-proliferative and proliferative diabetic retinopathy, diabetic nephropathy, diabetic cardiomyopathy, diabetic neuropathy, proliferative vitreoretinopathies, neovascular glaucoma, ischemic retinopathy, retinopathy secondary to retinal vein occlusion, as well as age-related macular degeneration, keloid formation, and wound healing. In embodiments, the kit is for use to detect a subject as having an increased probability of progressing to end-organ damage due to diabetes.

The kits can be used to detect the lncRNA biomarkers of the present invention. For example, the kits can be used to detect any one or more of the biomarkers described herein, which are differentially expressed in samples of a patient with the disease or condition. The kit may include one or more agents for detection of lncRNA biomarkers, a container for holding a biological sample isolated from a human subject; and printed instructions for reacting agents with the biological sample or a portion of the biological sample to detect the presence or amount of at least one lncRNA biomarker in the biological sample. The agents may be packaged in separate containers. The kit may further comprise one or more control reference samples and reagents for performing an immunoassay, a Northern blot, PCR, microarray analysis, or SAGE.

In certain embodiments, the kit contains at least one probe that selectively hybridizes to a biomarker, or at least one antibody that selectively binds to a biomarker, or at least one set of PCR primers for amplifying a biomarker. In one embodiment, the kit comprises at least one agent for measuring the level of HOTAIR.

In embodiments, the kit may include one or more HOTAIR inhibitors to treat the disease or condition listed above.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of diagnosing DR, including early stage DR or monitoring DR therapy.

The kits of the invention have a number of applications. For example, the kits can be used for monitoring DR progression. In another example, the kits can be used for evaluating the efficacy of a treatment for DR. In a further example, the kits can be used to identify compounds that modulate expression of one or more of the biomarkers in in vitro or in vivo animal models to determine the effects of treatment.

C. HOTAIR and Inhibitors

In another aspect, a HOTAIR inhibitor is used in the practice of the invention in the treatment of a disease or condition. Inhibitors of HOTAIR can include, but are not limited to, antisense oligonucleotides, inhibitory RNA molecules, such as miRNAs, siRNAs, piRNAs, and snRNAs, ribozymes, antibodies and small molecule inhibitors. Various types of inhibitors for inhibiting nucleic acid function are well known in the art. See e.g., International patent application WO/2012/018881; U.S. patent application 2011/0251261; U.S. Pat. No. 6,713,457; Kole et al. (2012) Nat. Rev. Drug Discov. 11(2):125-40; Sanghvi (2011) Curr. Protoc. Nucleic Acid Chem. Chapter 4:Unit 4.1.1-22; herein incorporated by reference in their entireties.

In one embodiment, the present invention is a method of treating a condition associated with overexpression of the long non-coding RNA HOTAIR, the method comprising: administering to a subject in need thereof a therapeutically effective amount of at least one agent that inhibits at least one biological activity of the long non-coding RNA HOTAIR. In embodiments, the disease or condition associated with overexpression of the long non-coding RNA HOTAIR is a diabetes-induced disease or condition. In embodiments, the disease or condition associated with overexpression of the long non-coding RNA HOTAIR is one or more of: diabetic retinopathy (including non-proliferative and proliferative diabetic retinopathy), diabetic nephropathy, diabetic cardiomyopathy, diabetic neuropathy, proliferative vitreoretinopathies, neovascular glaucoma, ischemic retinopathy, retinopathy secondary to retinal vein occlusion, as well as age-related macular degeneration, keloid formation, and wound healing. In embodiments, the disease or condition associated with overexpression of the long non-coding RNA HOTAIR is a condition in which anti-VEGF therapy is ineffective.

In another embodiment, the present invention provides for a method of treating a patient who does not respond to anti-VEGF therapy, the method comprising: administering to the subject in need thereof a therapeutically effective amount of at least one agent that inhibits at least one biological activity of the long non-coding RNA HOTAIR.

The present invention provides also for a method of preventing glucose-induced oxidative damage, the method comprising: administering to a subject in need thereof a therapeutically effective amount of at least one agent that inhibits at least one biological activity of the long non-coding RNA HOTAIR.

Inhibitors can be single stranded or double stranded polynucleotides and may contain one or more chemical modifications, such as, but not limited to, locked nucleic acids, peptide nucleic acids, sugar modifications, such as 2′-O-alkyl (e.g., 2′-O-methyl, 2′-O-methoxyethyl), 2′-fluoro, and 4′-thio modifications, and backbone modifications, such as one or more phosphorothioate, morpholino, or phosphonocarboxylate linkages. In addition, inhibitory RNA molecules may have a “tail” covalently attached to their 3′- and/or 5′-end, which may be used to stabilize the RNA inhibitory molecule or enhance cellular uptake. Such tails include, but are not limited to, intercalating groups, various kinds of reporter groups, and lipophilic groups attached to the 3′ or 5′ ends of the RNA molecules. In certain embodiments, the RNA inhibitory molecule is conjugated to cholesterol or acridine. See, for example, the following for descriptions of syntheses of 3′-cholesterol or 3′-acridine modified oligonucleotides: Gamper, H. B., Reed, M. W., Cox, T., Virosco, J. S., Adams, A. D., Gall, A., Scholler, J. K., and Meyer, R. B. (1993) Facile Preparation and Exonuclease Stability of 3′-Modified Oligodeoxynucleotides. Nucleic Acids Res. 21 145-150; and Reed, M. W., Adams, A. D., Nelson, J. S., and Meyer, R. B., Jr. (1991) Acridine and Cholesterol-Derivatized Solid Supports for Improved Synthesis of 3′-Modified Oligonucleotides. Bioconjugate Chem. 2 217-225 (1993); herein incorporated by reference in their entireties. Additional lipophilic moieties that can be used, include, but are not limited to, oleyl, retinyl, and cholesteryl residues, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine. Additional compounds, and methods of use, are set out in US Patent Publication Nos. 2010/0076056, 2009/0247608 and 2009/0131360; herein incorporated by reference in their entireties.

In one embodiment, inhibition of HOTAIR function may be achieved by administering antisense oligonucleotides targeting HOTAIR. The antisense oligonucleotides may be ribonucleotides or deoxyribonucleotides. Preferably, the antisense oligonucleotides have at least one chemical modification. Antisense oligonucleotides may be comprised of one or more “locked nucleic acids”. “Locked nucleic acids” (LNAs) are modified ribonucleotides that contain an extra bridge between the 2′ and 4′ carbons of the ribose sugar moiety resulting in a “locked” conformation that confers enhanced thermal stability to oligonucleotides containing the LNAs. Alternatively, the antisense oligonucleotides may comprise peptide nucleic acids (PNAs), which contain a peptide-based backbone rather than a sugar-phosphate backbone. The antisense oligonucleotides may contain one or more chemical modifications, including, but are not limited to, sugar modifications, such as 2′-O-alkyl (e.g. 2′-O-methyl, 2′-O-methoxyethyl), 2′-fluoro, and 4′ thio modifications, and backbone modifications, such as one or more phosphorothioate, morpholino, or phosphonocarboxylate linkages (see, for example, U.S. Pat. Nos. 6,693,187 and 7,067,641, which are herein incorporated by reference in their entireties). In some embodiments, suitable antisense oligonucleotides are 21-O-methoxyethyl “gapmers” which contain 21-O-methoxyethyl-modified ribonucleotides on both 5′ and 3′ ends with at least ten deoxyribonucleotides in the center. These “gapmers” are capable of triggering RNase H-dependent degradation mechanisms of RNA targets. Other modifications of antisense oligonucleotides to enhance stability and improve efficacy, such as those described in U.S. Pat. No. 6,838,283, which is herein incorporated by reference in its entirety, are known in the art and are suitable for use in the methods of the invention. Antisense oligonucleotides may comprise a sequence that is at least partially complementary to a HOTAIR target sequence, e.g., at least about 75%, 80%, 85%, 90%, 91%, 92%, 93%, 94%, 95%, 96%, 97%, 98%, or 99% complementary to the HOTAIR target sequence. In some embodiments, the antisense oligonucleotide may be substantially complementary to the HOTAIR target sequence, that is at least about 95%, 96%, 97%, 98%, or 99% complementary to a target polynucleotide sequence. In one embodiment, the antisense oligonucleotide comprises a sequence that is 100% complementary to the HOTAIR target sequence.

In another embodiment, the inhibitor of HOTAIR is an inhibitory RNA molecule (e.g., a miRNA, a siRNA, a piRNA, or a snRNA) having a single-stranded or double-stranded region that is at least partially complementary to the target sequence of HOTAIR, e.g., about 75%, 80%, 85%, 90%, 91%, 92%, 93%, 94%, 95%, 96%, 97%, 98%, or 99% complementary to the target sequence of HOTAIR. In some embodiments, the inhibitory RNA comprises a sequence that is substantially complementary to the target sequence of HOTAIR, e.g., about 95%, 96%, 97%, 98%, or 99% complementary to a target polynucleotide sequence. In other embodiments, the inhibitory RNA molecule may contain a region that has 100% complementarity to the target sequence. The inhibitory molecules may target the HOTAIR sequence. In certain embodiments, the inhibitory RNA molecule may be a double-stranded, small interfering RNA or a short hairpin RNA molecule (shRNA) comprising a stem-loop structure. In one embodiment, the HOTAIR inhibitor is an siRNA comprising a nucleotide sequence selected from the group consisting of SEQ ID NOS: 104, 106, 108 and 110.

An “effective amount” of a HOTAIR inhibitor (e.g., microRNA, siRNA, piRNA, snRNA, antisense oligonucleotide, ribozyme, or small molecule inhibitor) is an amount sufficient to effect beneficial or desired results, such as an amount that reduces HOTAIR activity, for example, by interfering with transcription of HOTAIR or interfering with the interaction of HOTAIR to its cellular targets. In some embodiments, a HOTAIR inhibitor reduces the amount and/or activity of HOTAIR by at least about 10% to about 100%, 20% to about 100%, 30% to about 100%, 40% to about 100%, 50% to about 100%, 60% to about 100%, 70% to about 100%, 10% to about 90%, 20% to about 85%, 40% to about 84%, 60% to about 90%, including any percent within these ranges, such as but not limited to 15%, 20%, 25%, 30%, 35%, 40%, 45%, 50%, 55%, 60%, 65%, 70%, 75%, 80%, 85%, 90%, 95%, 96%, 97%, 98%, and 99%.

In embodiments, the HOTAIR inhibitor is a siRNA. In embodiments, HOTAIR siRNA include SEQ ID NO:104, SEQ ID NO:106, SEQ ID NO:108, SEQ ID NO:110, N-187951-01, 187951-02, 187951-03, 187951-04.

In certain embodiments, the invention includes a method of modulating the expression of HOTAIR targets, the method comprising introducing into the cell an inhibitor of HOTAIR. In one embodiment, the activity of VEGF is reduced in the cell following administration of an inhibitor of HOTAIR.

In certain embodiments, the invention includes a method of modulating the expression of one or more apoptotic inhibitors that can be detectably labeled by well-known techniques. Detectable labels include, for example, radioactive isotopes, fluorescent labels, chemiluminescent labels, bioluminescent labels and enzyme labels. Such labeled inhibitors can be used to determine cellular uptake efficiency, quantitate binding of inhibitors at target sites, or visualize inhibitor localization.

In certain embodiments, the present invention includes a method of modulating, including inhibiting, the expression of one or more epigenetic molecules mediator. The epigenetic molecule may be one or more of EZH2, SUZ12, EED, DNMT1, DNMT3A, DNMT3B, CTCF and/or P300.

In certain embodiments, HOTAIR or a HOTAIR inhibitor is expressed in vivo from a vector. A “vector” is a composition of matter which can be used to deliver a nucleic acid of interest to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, lentiviral vectors, and the like. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In one embodiment, an expression vector for expressing HOTAIR or a HOTAIR inhibitor comprises a promoter “operably linked” to a polynucleotide encoding HOTAIR or a HOTAIR inhibitor. The phrase “operably linked” or “under transcriptional control” as used herein means that the promoter is in the correct location and orientation in relation to a polynucleotide to control the initiation of transcription by RNA polymerase and expression of the polynucleotide.

In certain embodiments, the nucleic acid encoding a polynucleotide of interest is under transcriptional control of a promoter. A “promoter” refers to a DNA sequence recognized by the synthetic machinery of the cell, or introduced synthetic machinery, required to initiate the specific transcription of a gene. The term promoter will be used here to refer to a group of transcriptional control modules that are clustered around the initiation site for RNA polymerase I, II, or III. Typical promoters for mammalian cell expression include the SV40 early promoter, a CMV promoter such as the CMV immediate early promoter (see, U.S. Pat. Nos. 5,168,062 and 5,385,839, incorporated herein by reference in their entireties), the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression. These and other promoters can be obtained from commercially available plasmids, using techniques well known in the art. See, e.g., Sambrook et al., supra. Enhancer elements may be used in association with the promoter to increase expression levels of the constructs. Examples include the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777 and elements derived from human CMV, as described in Boshart et al., Cell (1985) 41:521, such as elements included in the CMV intron A sequence.

Typically, transcription terminator/polyadenylation signals will also be present in the expression construct. Examples of such sequences include, but are not limited to, those derived from SV40, as described in Sambrook et al., supra, as well as a bovine growth hormone terminator sequence (see, e.g., U.S. Pat. No. 5,122,458). Additionally, 5′-UTR sequences can be placed adjacent to the coding sequence in order to enhance expression of the same. Such sequences include UTRs which include an Internal Ribosome Entry Site (IRES) present in the leader sequences of picornaviruses such as the encephalomyocarditis virus (EMCV) UTR (fang et al. J. Virol. (1989) 63:1651-1660. Other picornavirus UTR sequences that will also find use in the present invention include the polio leader sequence and hepatitis A virus leader and the hepatitis C IRES.

In certain embodiments of the invention, the cells containing nucleic acid constructs of the present invention may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Fluorescent markers (e.g., green fluorescent protein (GFP), EGFP, or Dronpa), or immunologic markers can also be employed. The selectable marker employed is not believed to be important, so long as it is capable of being expressed simultaneously with the nucleic acid encoding a gene product. Further examples of selectable markers are well known to one of skill in the art.

There are a number of ways in which expression vectors may be introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986).

One of the preferred methods for in vivo delivery involves the use of an adenovirus expression vector. “Adenovirus expression vector” is meant to include those constructs containing adenovirus sequences sufficient to (a) support packaging of the construct and (b) to express a polynucleotide that has been cloned therein. The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

The typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors, as described by Karlsson et al. (1986), or in the E4 region where a helper cell line or helper virus complements the E4 defect.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

Retroviral vectors are also suitable for expressing HOTAIR or HOTAIR inhibitors in cells. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Porter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding HOTAIR or the HOTAIR inhibitor of interest may be positioned and expressed at different sites. In certain embodiments, the nucleic acid encoding HOTAIR or a HOTAIR inhibitor may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ohosh and Bachhawat, 1991). Also contemplated are lipofectamine-DNA complexes.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-I) (Kato et al., 1991). In yet further embodiments, the liposome may be complexed or employed in conjunction with both HVJ and HMG-I. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular lncRNA or inhibitor into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, vascular endothelial growth factor (VEGF) may be used as the receptor for mediated delivery of a nucleic acid into cells that exhibit upregulation of VEGF receptor.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

The present invention also encompasses pharmaceutical compositions comprising one or more HOTAIR inhibitors and a pharmaceutically acceptable carrier. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

Colloidal dispersion systems, such as macromolecule complexes, nanocapsules, microspheres, beads, and lipid-based systems including oil-in-water emulsions, micelles, mixed micelles, and liposomes, may be used as delivery vehicles for HOTAIR inhibitors described herein. Commercially available fat emulsions that are suitable for delivering the nucleic acids of the invention to tissues, such as cardiac muscle tissue and smooth muscle tissue, include Intralipid, Liposyn, Liposyn II, Liposyn III, Nutrilipid, and other similar lipid emulsions. A preferred colloidal system for use as a delivery vehicle in vivo is a liposome (i.e., an artificial membrane vesicle). The preparation and use of such systems is well known in the art. Exemplary formulations are also disclosed in U.S. Pat. Nos. 5,981,505; 6,217,900; 6,383,512; 5,783,565; 7,202,227; 6,379,965; 6,127,170; 5,837,533; 6,747,014; and WO 03/093449, which are herein incorporated by reference in their entireties.

One will generally desire to employ appropriate salts and buffers to render delivery vehicles stable and allow for uptake by target cells. Buffers also will be employed when recombinant cells are introduced into a patient. Aqueous compositions of the present invention comprise an effective amount of the delivery vehicle, dissolved or dispersed in a pharmaceutically acceptable carrier or aqueous medium. The phrases “pharmaceutically acceptable” or “pharmacologically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the nucleic acids of the compositions.

The pharmaceutical forms suitable for injectable use or catheter delivery include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present invention generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like).

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present invention. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by FDA Office of Biologics standards.

### D. Administration

At least one therapeutically effective dose of a HOTAIR inhibitor will be administered. The HOTAIR inhibitor may be an antisense oligonucleotide or inhibitory RNA molecule such as, a miRNA, siRNA, piRNA, or snRNA, or a ribozyme, as described herein.

By “therapeutically effective dose or amount” of each of these agents is intended an amount that when administered in combination brings about a positive therapeutic response with respect to treatment of an individual for diabetes.

The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case.

In certain embodiments, multiple therapeutically effective doses of each of at least one HOTAIR inhibitor will be administered according to a daily dosing regimen, or intermittently By “intermittent” administration is intended the therapeutically effective dose can be administered, for example, every other day, every two days, every three days, and so forth. For example, in some embodiments, at least one HOTAIR inhibitor will be administered twice-weekly or thrice-weekly for an extended period of time, such as for 1, 2, 3, 4, 5, 6, 7, 8 . . . 10 . . . 15 . . . 24 weeks, and so forth.

In other embodiments of the invention, the pharmaceutical compositions comprising the agents, such as one or more HOTAIR inhibitors, is a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The pharmaceutical compositions comprising one or more HOTAIR inhibitors agents may be administered using the same or different routes of administration in accordance with any medically acceptable method known in the art. Suitable routes of administration include intravitreal administration, parenteral administration, such as subcutaneous (SC), intraperitoneal (IP), intramuscular (IM), intravenous (IV), or infusion, oral and pulmonary, nasal, topical, transdermal, and suppositories. Where the composition is administered via pulmonary delivery, the therapeutically effective dose is adjusted such that the soluble level of the agent, such as the HOTAIR inhibitor in the bloodstream, is equivalent to that obtained with a therapeutically effective dose that is administered parenterally.

Factors influencing the respective amount of the various compositions to be administered include, but are not limited to, the mode of administration, the frequency of administration (i.e., daily, or intermittent administration, such as twice- or thrice-weekly), the particular disease undergoing therapy, the severity of the disease, the history of the disease, whether the individual is undergoing concurrent therapy with another therapeutic agent, and the age, height, weight, health, and physical condition of the individual undergoing therapy. Generally, a higher dosage of this agent is preferred with increasing weight of the subject undergoing therapy.

Where a subject undergoing therapy in accordance with the previously mentioned dosing regimens exhibits a partial response, or a relapse following a prolonged period of remission, subsequent courses of concurrent therapy may be needed to achieve complete remission of the disease. Thus, subsequent to a period of time off from a first treatment period, a subject may receive one or more additional treatment periods comprising a HOTAIR inhibitor, which can be administered in combination with any other agent for the treatment of diabetes.

### E. Kits

Any of the compositions described herein may be included in a kit. For example, at least one HOTAIR inhibitor, and/or at least one anti-diabetes agent, or any combination thereof, may be included in a kit. The kit may also include one or more transfection reagents to facilitate delivery of oligonucleotides or polynucleotides to cells.

The components of the kit may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include a means for containing the nucleic acids, and any other reagent containers in close confinement for commercial sale. Such containers may include injection or blow-molded plastic containers into which the desired vials are retained.

A kit will also include instructions for employing the kit components as well the use of any other reagent not included in the kit. Instructions may include variations that can be implemented. A kit may also include utensils or devices for administering a HOTAIR inhibitor by various administration routes, such as parenteral or catheter administration or coated stent.

The present invention provides also for a use of the at least one agent that inhibits HOTAIR in the manufacture of a medicament for the treatment of diabetes-induced vascularization.

### EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Methods

### Cell Culture

Human retinal microvascular endothelial cells (HRECs; Cell Systems, Kirkland, Wash., USA; catalog number ACBRI 181), mouse retinal microvascular endothelial cells (MRECs; Applied Biological Materials Inc., Richmond, BC, CAN), and primary lung endothelial cells (MLECs) from C57BL/6J mice were cultured in endothelial basal media-2 (EBM-2, Lonza, Walkersville, Md., USA) containing endothelial growth media-2 (EGM-2) SingleQuots (Lonza). All cells were grown in 75 cm² culture flasks and maintained in a humidified incubator containing 5% CO₂ at 37° C. As described previously (22,55,56), in order to reduce variability for experimentation, cells were used between passages three and six and the cellular densities were determined accordingly based on the type of culture plates used for each experiment. Generally, once 80% confluence was obtained post-seeding, ECs were cultured in serum and growth factor-free medium overnight before exposure to different D-glucose levels (final glucose concentrations of 5 mmol/L, mimicking normoglycemia [NG], and 25 mmol/L, mimicking hyperglycemia [HG]) for various durations; the selected glucose levels are based on a large volume of previous experiments (22,27,33,55-57). All in vitro or ex vivo experiments were independently repeated at least three times and performed with six replicates, unless specified.

siRNA Transfections

HRECs were transfected using scrambled siRNAs (ID number: AM4635 [SCR], Thermo Fisher Scientific) or pre-designed siRNAs targeting human HOTAIR (ID: n272221 [si1-HOTAIR], Thermo Fisher Scientific; n272222 [si2-HOTAIR], Thermo Fisher Scientific; R-187951-00-0005 [SMARTpool siHOTAIR], Horizon Discovery; Table 6, top panel), EZH2 (ID: M-004218-03-0005 [SMARTpool si-EZH2], Horizon Discovery), CTCF (ID: M-020165-02-0005 [SMARTpool siCTCF], Horizon Discovery), DNMT1 (ID: s4216 [siDNMT1], Thermo Fisher Scientific) or mouse Hotair (ID: R-173526-00-0005 [SMARTpool siHOTAIR], Horizon Discovery, Table 6, bottom panel) using Lipofectamine 2000 (Invitrogen, Burlington, ON, Canada) and Opti-MEM reduced serum media (Thermo Fisher Scientific). As documented previously by us (22,27,33,55-57), cells were transfected with 100 nM of each siRNA for 3-4 hours and subsequently recovered in complete EBM-2 overnight. Cells were then serum starved the following morning, between 18-24 hours, and then incubated with specific glucose concentrations (5 mmol/L or 25 mmol/L) for 48 hours. Knockdown of the target genes were then confirmed using RT-qPCR.

### Enzyme-Linked Immunosorbent Assay (ELISA)

Human VEGF-A (R&D Systems, Minnesota, USA) ELISA kit was used to measure the cytokine levels from HREC supernatants. Cytokine concentrations were quantified using the BCA protein assay kit (Pierce, Rockford, Ill., USA) and 100 μg protein concentrations were used for the ELISA kit according to the manufacturer's instructions. The optical density for each well was determined at 450 nm and corrected at 568 nm using the Multiskan FC Microplate Photometer (Thermo Fisher Scientific, Massachusetts, USA).

### Endothelial Tube Formation Assay

When performing the tube formation assay, ˜1.5×10⁴ HRECs (pre-treated with either SCR siRNA or siHOTAIR) were seeded into a 96-well plate containing 100 μL of BD Phenol red-free matrigel matrix (BD Biosciences, Bedford, Mass., USA) per well. In the presence of growth medium, cells were allowed to attach for one hour in a humidified incubator with 5% CO₂ at 37° C. Following one-hour incubation, the growth medium was replaced with serum-free medium containing appropriate glucose concentrations (5 mM or 25 mM) and/or exogenous VEGF-A protein concentrations (50 ng/mL). At the six-hour mark, images were taken at a 40× magnification using the Nikon Diaphot microscope (Nikon Canada, Mississauga, ON, CAN) with a PixeLINK camera (PixeLINK, Ottawa, ON, CAN) and images were captured from at least two field views per well (n=8 independent samples/group). In order to assess the total number of tubules and branching points in the images, the WimTube Image analyzer software (Wimasis) was used and these results were plotted graphically.

### RNA Fluorescence In Situ Hybridization (RNA-FISH)

As previously described (56,57), HRECs were seeded at 75% confluency on glass cover slips in 12-well plates, serum starved over night, and treated with various glucose concentrations (NG or HG) for 48 hours. RNA fluorescence in situ hybridization (FISH) was performed according to the manufacturer's protocol for adherent cells (https://www.biosearchtech.com/support/resources/stellaris-protocols) and Stellaris FISH probes for Human HOTAIR with Quasar 570 dye (5 nmol; Biosearch Technologies, Petaluma, Calif., USA) were used for hybridization. HRECs were also counterstained with 4′,6-diamidino-2-phenylindole (DAPI; Vector Laboratories, Burlingame, Calif., USA) and mounted with Vectashield mounting medium (Vector Laboratories). Images were captured with the Cytation 5 Cell Imaging Multi-Mode Reader (BioTek, Winooski, Vt., USA), at a magnification of 20×, by researchers blinded to the experimental groups. Yellow fluorescent protein (YFP), DAPI, and phase contrast filters were used and images were subsequently analyzed using ImageJ software (NIH, Bethesda, Md., USA).

### RNA Immunoprecipitation (RIP)

Cell lysates from HRECs cultured in NG or HG were collected for immunoprecipitation at the 48-hour mark using the Magna RIP RNA-binding protein immunoprecipitation kit (Millipore, Etobicoke, ON, CAN) (27,57), following the manufacturer's instructions. Anti-IgG (control) and anti-EZH2 antibodies (Millipore) were used to co-precipitate the RNA-binding proteins of interest. The extracted RNAs were then reversed transcribed to cDNA, analyzed by RT-qPCR and normalized to the levels of B-actin mRNA (encoding a housekeeping protein).

3-Deazaneplanocin A (DZNep), 5-Aza-2′-deoxycytidine (5-aza-dC) and 2-deoxy-D-glucose (2-DG) Treatments

Following the concentrations documented in previous studies, DZNep (Cayman Chemical, Ann Arbor, Mich., USA; 5 μM), 5-aza-dC (Sigma, St. Louis, USA; 5 μM) or 2-DG (Sigma; 0.6 mM and 5 mM) pre-treatment was applied to HRECs for 1 hour prior to the addition of D-glucose (22,27,55,58). DZNep, 5-aza-dC or 2-DG-treated HRECs and their respective controls were collected at 48 hours for further analyses.

### JC-1 Assay

In order to assess mitochondrial health and functional status, the JC-1 assay was employed (33). Briefly, HRECs were treated with either SCR siRNA or siHOTAIR prior to glucose culture and at the 48-hour mark, cells were subsequently incubated for 10 minutes with 10 μM of the JC-1 dye (5,5′,6,6′-tetrachloro-1,1′,3,3′-tetraethylbenzimi-dazolylcarbocyanine iodide; Abcam, Toronto, ON, CAN). Following the manufacturer's instructions, HRECs were then washed three times using the JC-1 dilution buffer. In order to stain the nuclear regions, DAPI (Vector Laboratories) was used following JC-1 staining. Fluorescence images were captured at 20× magnification using the Zeiss LSM 410 inverted laser-scanning microscope (Carl Zeiss Canada, North York, ON, CAN) and images were analyzed using ImageJ.

### 8-OH-dG Staining

Following siRNA pre-treatment, HRECs were plated in eight-chamber tissue culture slides and incubated for 48 hours after glucose challenge (NG or HG) (33). Following the manufacturer's instructions, cells were fixed with methanol and then stained for 8-hydroxy-2′-deoxyguanosine (an oxidative DNA damage marker; 8-OHdG; 1:50, Santa Cruz Biotechnology, Dallas, Tex., USA). Nuclear staining was also performed using DAPI (Vector Laboratories). Microscopy was performed by a blinded examiner, who was unaware of the identity of the samples, using a Zeiss LSM 410 inverted laser scan microscope (Carl Zeiss Canada) and the images were captured at a 20× magnification and subsequently analyzed using ImageJ.

### Chromatin Immunopreciation-qPCR (ChIP-qPCR)

ChIP assays (Milipore, Temecula, Calif., USA) were carried out as previously described by us (55). Briefly, HRECs were pre-treated with either SCR siRNA or siHOTAIR and subsequently cultured in NG or HG for 48 hours. Cells were then fixed with 1% formaldehyde, incubated for 10 minutes at 37° C., and then lysed and sonicated to shear DNA. ChIP assays were performed using anti-trimethyl-Histone H3 (Lys27; H3k27me3; Millipore), anti-RNA polymerase II (Millipore), anti-IgG (Millipore) and anti-acetyl-Histone H3 (K9, K14, K18, K23, K27; Abcam) antibodies. Anti-mouse IgG was used as a negative control. The immunoprecipitated DNA was detected by RT-qPCR using promoter-specific primers for VEGF-A: distal promoter region (forward: 5′-GTAGTCCCAGGGTGCAACAC-3′ (SEQ ID NO: 111), reverse: 5′-GACTGGCTAGAATGGGCATC-3′ (SEQ ID NO: 112), location relative to transcriptional start site [TSS]: −4896 bp) and proximal promoter region (forward: 5′-CGGTGCTGGAATTTGATATTCATTGAT-3′ (SEQ ID NO: 113), reverse: 5′-TTCAAGTGGGGAATGGCAAGC-3′ (SEQ ID NO: 114), location relative to TSS: −189 bp) (59).

### WST-1 Cell Viability & Proliferation Assay

Following glucose and siHOTAIR treatments, the viability of HRECs was determined using the WST-1 Cell Viability Assay (Roche) at 48 hours. Using the Multiskan FC Microplate Photometer (Thermo Fisher Scientific), absorbances were first measured at 450 nm and then corrected using 690 nm as the reference wavelength.

### Electron Microscopy

Following the transfection of HRECs on coverslips, the inserts with attached cells were fixed in 2.5% glutaraldehyde in phosphate buffer and processed for EPON embedding as previously described (60). Ultra-thin sections on 200 mesh nickel grids were stained with uranyl acetate and lead citrate and examined electronmicroscopically (Phillips EM-420 TEM).

### Methylation Analysis of CpG Sites Across HOTAIR

Differential methylation patterns of CpG sites across the HOTAIR gene were identified in HRECs, incubated in NG or HG environments for 2 days (48 hours) or 7 days (168 hours), using the Illumina Infinium MethylationEPIC BeadChip array (Illumina, Calif., USA). At each respective time-point, genomic DNA was extracted from these cells and 1 μg of DNA was used for bisulfite conversion using the Blood & Cell Culture DNA Mini Kit (Qiagen, Toronto, ON, CAN). The HiScan System (Illumina, Calif., USA) was used to obtain the array readout and the methylated and unmethylated signal intensity data were then imported into R 3.5.2 for analysis. Following our previously published protocols (61), normalization was performed using the Illumina normalization method with background correction using the minfi package. Probes with detection p-value >0.01 were excluded from the downstream analysis. In addition, probes known to contain single nucleotide polymorphisms (SNPs) at the CpG interrogation or the single nucleotide extension were removed. Methylation level for each probe was measured as a beta value (β-value), calculated from the ratio of the methylated signals versus the total sum of unmethylated and methylated signals, ranging between 0 (no methylation) and 1 (full methylation). Three independent samples were used per group.

### Diabetic Animal Models

The Western University Council for Animal Care Committee approved all animal models used in this study and experiments were performed in accordance with The Guide for the Care and Use of Laboratory Animals (NIH Publication 85-23, revised in 1996). Beginning with our initial two-month in vivo model, male rats (Sprague-Dawley; ˜175 g, 6 weeks old) or male mice (C57/BL6 background; ˜25 g, 8 weeks old) were obtained (Charles River, Wilmington, Mass., USA) and randomly divided into control and diabetic groups. Streptozotocin (STZ) was used to generate a type 1 diabetic animal model and methods of diabetes induction and monitoring have been previously described (27,56,57). At two months following diabetes induction, animals were euthanized (n=8 for both mice groups; n=5 for control rats and n=9 for diabetic rats) and retinal tissues were collected for RNA extraction and Hotair RNA levels were assessed using RT-qPCR.

For our short-term therapeutic in vivo model (4-week duration), wild-type mice were obtained (Charles River; C57BL/6J background; ˜25 g, 8 weeks old) and randomly divided into four groups (n=6/group): control mice administered intravitreal injections of SCR siRNA (negative control) or siHOTAIR, and diabetic mice administered intravitreal injections of SCR siRNA or siHOTAIR. Prior to administering intravitreal injections, STZ-induced diabetes was first confirmed in diabetic animals (>20 mmol/L blood glucose levels) using the above methodologies. After the onset of diabetes, a 1 μl solution (100 nmol/L) containing either SCR siRNA or siHOTAIR with Lipofectamine 2000 (Invitrogen) was injected into the vitreous chamber of the diabetic mice eye once every week for up to three weeks. Control mice were also injected similarly with the same volume of SCR siRNA or siHOTAIR with Lipofectamine. All mice were anaesthetized using isoflurane (2.25% mixed with 900 mL/min O₂) and intravitreal injections were performed with a 33-gauge needle attached to a 10 μl glass syringe (Hamilton, Reno, USA). Surgical positioning of the needle and the general duration of each intravitreal injection have been described previously (62). No post-surgical ocular complications occurred throughout the 4-week study.

### Toxicity and Histopathological Analyses

To determine potential adverse effects of siHOTAIR in mice, toxicity analyses were performed in addition to the regular monitoring of mice (C57/BL6 background) (64). Age-matched mice were divided into four groups: SCR siRNA group (negative control; 100 nmol/L; n=3), siHOTAIR low-dose group (25 nmol/L; n=3), siHOTAIR middle-dose group (50 nmol/L; n=3) and siHOTAIR high-dose group (100 nmol/L; n=3). SCR siRNA or siHOTAIR were intravitreally injected once, as a single-dose, and mice were followed-up for 7 consecutive days. Following this time-point, organs were excised, fixed in 10% buffered formalin solution and embedded, and sectioned into 5 μm thick sections. The tissue sections were then stained with hematoxylin and eosin (H&E) for routine histology. A blinded pathologist evaluated the histopathological damage using a light microscope and the images were captured (Nikon, Japan). Of note, in order to examine long-term toxicity of siHOTAIR, we had also performed H&E staining for the mice tissues obtained from our 4-week therapeutic model (n=3/group).

### Clinical Sample Collection

The Western Research Ethics Board and Lawson Health Research Institute at the University of Western Ontario (London, ON, CAN) approved the clinical component of this study. Patients provided informed consent prior to the procurement of specimens and all of the samples were handled in accordance with the Declaration of Helsinki. Both serum and undiluted vitreous humor (VH) were collected from patients undergoing a pars plana vitrectomy by an experienced vitreoretinal surgeon. Both specimens were categorized into two groups: control and proliferative diabetic retinopathy (PDR). The PDR group comprised of patients diagnosed with advanced stages of DR (PDR; n=11; mean age ±SD=60.7±10.72 years; 10 males and 1 female), while the control group consisted of patients that had no previous history of PDR and were diagnosed with idiopathic macular hole or a separate non-diabetic ocular condition (n=10; mean age ±SD=69.2±8.87 years; 2 males and 8 females). PDR was defined as the presence of neovascularization or fibrous proliferation of the disc or elsewhere on the retina. As previously described (27,56), total RNA was extracted from 500 μL of VH samples and 200 μL of serum samples using the TRIzol reagent (Invitrogen) and a serum RNA extraction kit (Bio Basic Inc., Markham, ON, CAN) following the manufacturer's protocol. After conversion to cDNA, RT-qPCR was used to evaluate the expression of HOTAIR in these samples.

### RNA Isolation and Quantitative Real-Time Polymerase Chain Reaction (RT-qPCR)

As extensively described by us (22,27,33,55-57), total RNA was extracted using the TRIzol reagent (Invitrogen). Once total RNA was obtained, a spectrophotometer (260 nm; Gene Quant, Pharmacia Biotech, USA) was used to quantify RNA concentrations in which 1-2 μg of total RNA was reverse transcribed to complementary DNA (cDNA) using a high-capacity cDNA reverse-transcription kit (Applied Biosystems/Thermo Fisher Scientific). cDNA was then amplified in the LightCycler 96 System (Roche Diagnostics, Laval, QC, CAN) using the SYBR-green master mix (Takara Bio, Mountain View, Calif., USA) and specific primers for the genes of interest (Sigma; Tables 1-2). RT-qPCR results were analyzed using the LightCycler 96 SW 1.1 software (Roche) and expression levels were calculated by the relative standard curve method using β-actin as an internal control for sample normalization.

### Statistical Analyses

Statistical differences were evaluated between groups using GraphPad Prism 7 (La Jolla, Calif., USA). Data were considered statistically significant if the P value was less than 0.05. All quantitative data for the in vitro experiments are presented as mean±SEM, while all in vivo data are presented as mean±SD. Experiments were performed in triplicate (n=6 per group), unless specified. Statistical significance for samples with non-parametric distribution was identified using the Mann-Whitney U test, while two-tailed Student's t-test (when comparing two conditions) or one-way ANOVA (for multiple comparisons; followed by Tukey's post hoc test) was applied for parametric variables.

### Results

### HOTAIR RNA Expressions are Glucose-Dependent

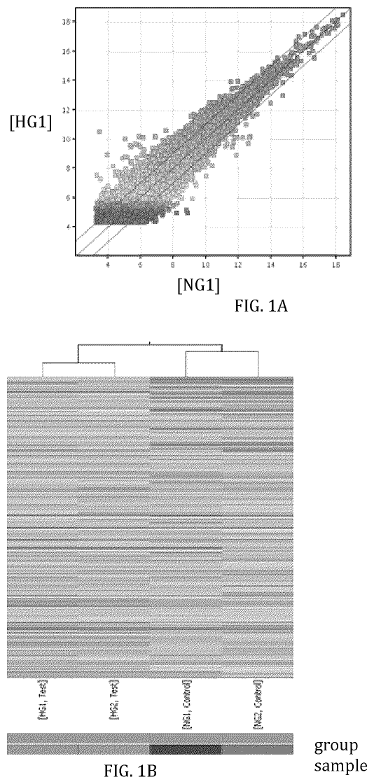

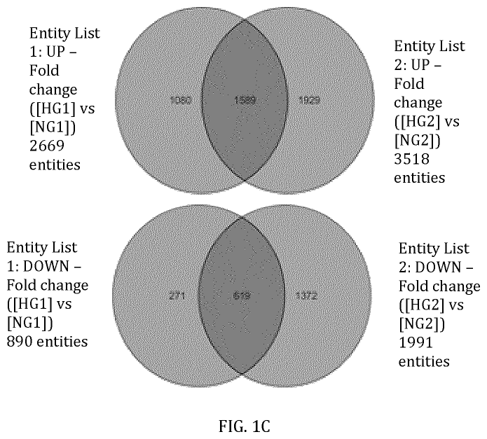

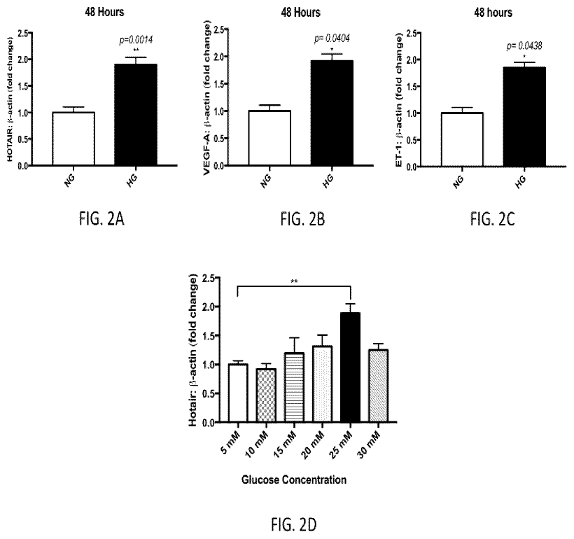

Using microarray analyses, we previously explored the global expression profiles of lncRNAs in Human Retinal Endothelial Cells (HRECs) cultured with NG or HG for 48 hours (Gene Expression Omnibus [GEO] ID: GSE122189). Interestingly, following stringent filtering criteria (fold change ≤ or ≥2 and an adjusted p-value <0.05), thousands of lncRNAs were differentially expressed after HG glucose culture; in particular, when examining between replicates, 2669-3518 lncRNAs were found to be upregulated and 890-1991 lncRNAs were found to be downregulated in HRECs challenged with HG (FIGS. 1A-C). Among the upregulated lncRNAs, the lncRNA HOTAIR was increased by 2.67-fold in HG-treated HRECs compared to NG controls (Table 3). Real-time quantitative reverse transcription-PCR (RT-qPCR) further confirmed the elevated expressions of HOTAIR following 48 hours of HG culture in HRECs (FIG. 2A), which were also associated with augmented expressions of VEGF-A and ET-1 transcripts (FIGS. 2B and 2C). Since lncRNAs have been reported to demonstrate differential expression patterns across various time-points (27,63), we investigated HOTAIR RNA expressions at 6, 12, 24, 48, and 72 hours (data not shown). Intriguingly, when compared to their respective NG controls, HOTAIR demonstrated significant HG-induced elevations only at the 48-hour (FIG. 2A; p=0.0014). Furthermore, to determine whether specific glucose concentrations can influence HOTAIR expressions, we cultured HRECs in the presence of 5, 10, 15, 20, and 25 mmol/L (mM) D-glucose for 48 hours. As demonstrated by RT-qPCR, HOTAIR RNA expressions peaked significantly following 25 mM glucose culture (mimicking hyperglycemia) compared to cells cultured with 5 mM glucose (mimicking euglycemia) (p=0.0077; FIG. 2D). Therefore, based on the present findings and our previously published studies (22,27,55-57), the 48-hour time-point and 5 mM and 25 mM glucose concentrations were used for our subsequent in vitro experiments. Of note, for the above experiments, no significant differences in HOTAIR expressions were observed when using an osmotic control (25 mM L-glucose; data not shown).

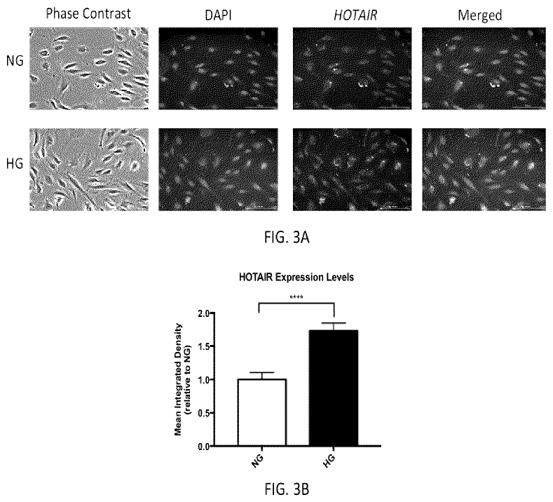

To delineate the sub-cellular localization of HOTAIR in HRECs, we performed RNA fluorescence in situ hybridization (RNA FISH). RNA FISH showed that HOTAIR can be present in both nuclear and cytoplasmic compartments of HRECs, with a predominant localization in the perinuclear/cytosolic region (FIG. 3A). Moreover, further confirming our microarray and RT-qPCR findings, RNA FISH analyses demonstrated that HG significantly promotes elevated expressions of HOTAIR when compared to NG controls (FIG. 3B; p<0.0001). Taken together, these data reveal that HG is an inducer of HOTAIR expressions and additionally imply an endothelial-specific role for HOTAIR during HG stress, where HOTAIR may be involved in the regulation of nuclear and cytoplasmic processes.

### HOTAIR Directly Mediates Angiogenesis in Hyperglycemic Environments In Vitro

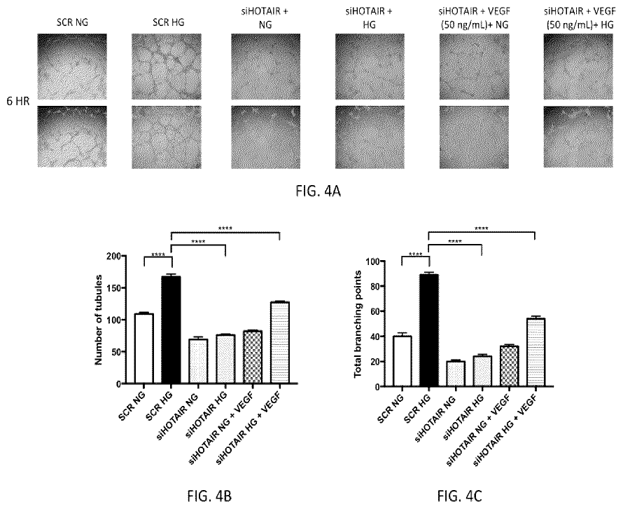

In order to determine HOTAIR's angiogenic role, we used HRECs and performed an endothelial cell tube formation assay, which is a widely used in vitro assay that models the reorganization stage of angiogenesis and is a rapid method that can determine genes or pathways involved in angiogenesis. As evident by the images in FIG. 4A, at the 6-hour mark, cells pre-treated with scrambled siRNAs (denoted as ‘SCR’) and cultured in the presence of HG have an elevated presence of capillary-like structures (tubules) compared to pre-treated SCR cells incubated in NG. However, when cells were treated with siHOTAIR, the degree of branching and total number of tubules significantly decreased in both NG and HG conditions at 6 hours (FIGS. 4B and 4C; p<0.0001). Even more intriguing at the 6-hour mark, when HRECs were pre-treated with siHOTAIR, the presence of both exogenous VEGF proteins and HG were not able to completely recover the degree of branching and number of tubules compared to HG controls, which implies that the knockdown of HOTAIR may be further desensitizing ECs to other external angiogenesis-causing factors in HG. These findings encouraged us to explore other angiogenic factors (64), such as angiopoietin-like 4 (ANGPTL4), placental growth factor (PGF), hypoxia-inducible factor (HIF), interleukin-1 beta (IL-113), and diabetes-related molecules including poly [ADP-ribose]-polymerase 1 (PARP1) (65), Cytochrome B (33), and several additional epigenetic mediators in the next set of experiments below.

### HOTAIR Knockdown can Prevent the Induction of Several Angiogenic Factors and Diabetes-Related Molecules In Vitro

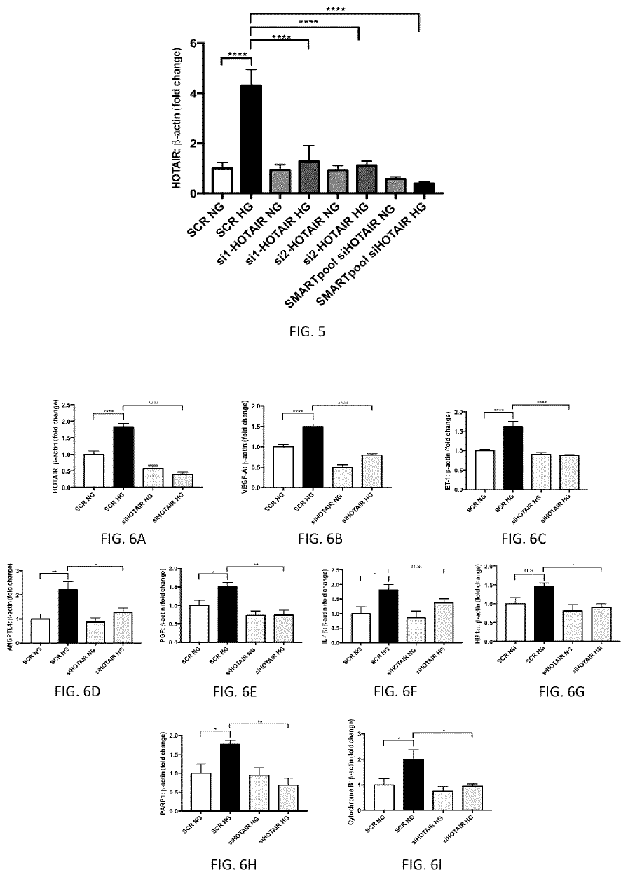

To determine the direct regulatory capabilities of HOTAIR on the aforesaid molecules of pathogenetic significance in DR in vitro, we carried out a loss-of-function experiment that involved siRNA-mediated knockdown of HOTAIR in HRECs. Amongst the three siRNAs tested and albeit significant decreased HOTAIR expressions were observed across all siRNA treatments, the SMARTpool siHOTAIR′ evoked the largest reduction of HOTAIR RNA levels (by ˜91%, p<0.0001) in HG-cultured HRECs compared to HG SCR controls (FIG. 5). Therefore, as such, we selected the SMARTpoor siRNA for our subsequent downstream analyses.

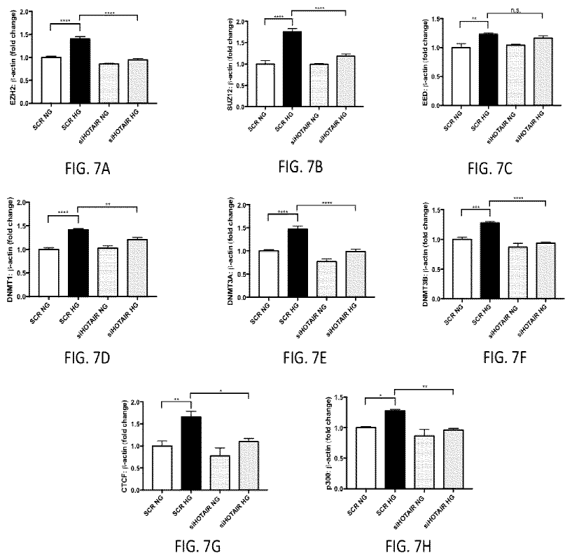

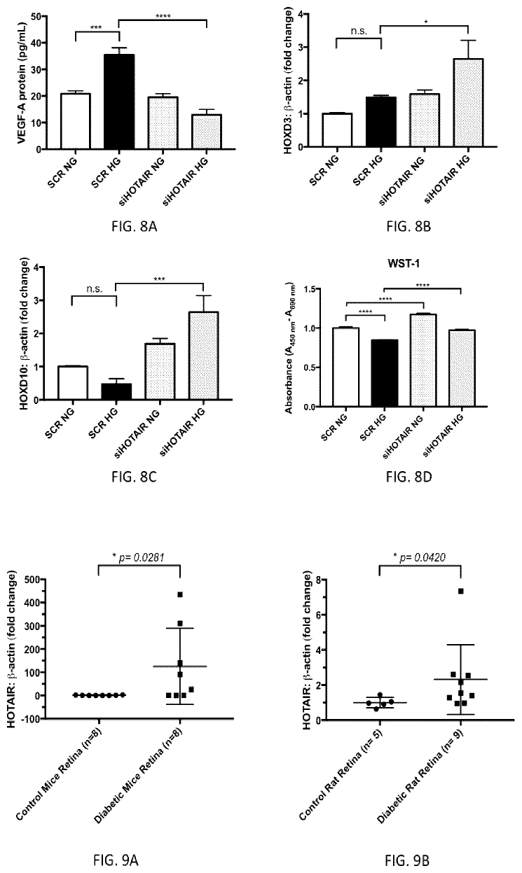

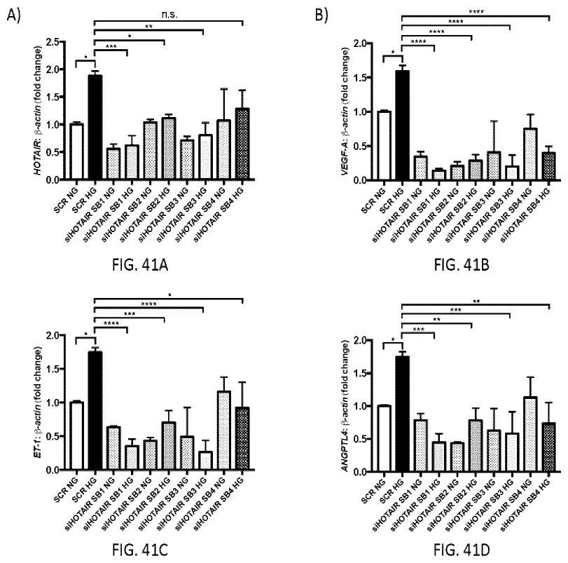

Accompanying the reduced HOTAIR levels in siHOTAIR-treated HRECs challenged with HG, significantly decreased expressions of various RNA transcripts implicated in angiogenesis (VEGF-A, ET-1, ANGPTL4, PGF, HIF-la; FIGS. 6A-G), DNA and oxidative damage (PARP-1 and Cytochrome B; FIGS. 6H and 6I), and epigenetic regulation (EZH2, SUZ12, DNMT1, DNMT3A, DNMT3B, CTCF, and P300; FIG. 7A-H) were also evident when compared to HG controls. These findings indicate that the lncRNA HOTAIR is directly implicated in the transcriptional regulation of several DR-related molecules. To determine whether these molecular changes are also reflected at the protein level, we selected one of the angiogenic markers (VEGF-A) for further follow-up via ELISA. In parallel to our RNA results, the knockdown of HOTAIR can significantly prevent glucose-induced upregulations of VEGF-A proteins in HRECs (FIG. 8A; p<0.0001). Extending our findings, we had additionally examined the expressions of HOXD3 and HOXD10, since HOTAIR has been implicated in the transcriptional repression of HOXD loci (66). Indeed, the knockdown of HOTAIR in HRECs cultured with HG can induce significant upregulations of HOXD3 (p=0.0473) and HOXD10 (p=0.0001) compared to SCR HG controls (FIGS. 8B and 8C). Furthermore, custom double-stranded siRNAs targeting specific regions of HOTAIR (near 5′ end, middle gene body, and near 3′ end) were developed and HRECs were subsequently transfected. Compared to SCR HG controls (FIG. 41), HOTAIR expressions were reduced by ˜67%, ˜41%, ˜57%, and ˜32% using siHOTAIR SB1, siHOTAIR SB2, siHOTAIR SB3, and siHOTAIR SB4, respectively. The knockdown of HOTAIR also directly influenced the transcript levels of VEGF-A, ET-1 and ANGPTL4, albeit variable reductions existing for each siRNA (greatest reductions were observed for the siRNAs designed to target near the 5′ end of HOTAIR: SB1 and SB2). We had also investigated the viability of HRECs following siHOTAIR treatment and as evidenced by our WST-1 findings, siRNA-mediated knockdown of HOTAIR can significantly improve cellular viability compared to SCR controls (FIG. 8D; p<0.0001). Collectively, these results suggest that HOTAIR is a critical regulator of glucose-induced EC dysfunction in vitro.

### HOTAIR is Significantly Elevated in the Retinas of Diabetic Mice and Rats at 2 Months

Following our in vitro findings, we wanted to confirm in vivo whether HOTAIR had a similar pathogenetic phenotype in the retina in diabetes. As such, we employed a streptozotocin (STZ)-induced diabetic animal model involving both C57/BL6 mice and Sprague-Dawley rats and subsequently extracted retinal tissues after 2 months of diabetes. Diabetic animals showed hyperglycemia and glucosuria, as well as reduced body weight gain and hyperglycemia (data not shown). In parallel to the trends observed from our in vitro experiments, distinct patterns of Hotair RNA expressions were evident between the retinas of control and diabetic animals, with significant upregulations of Hotair in the retinas of both diabetic mice (p=0.0281; FIG. 9A) and rats (p=0.0420; FIG. 9B) at 2 months, suggesting that retinal Hotair expressions share a positive association with diabetes.

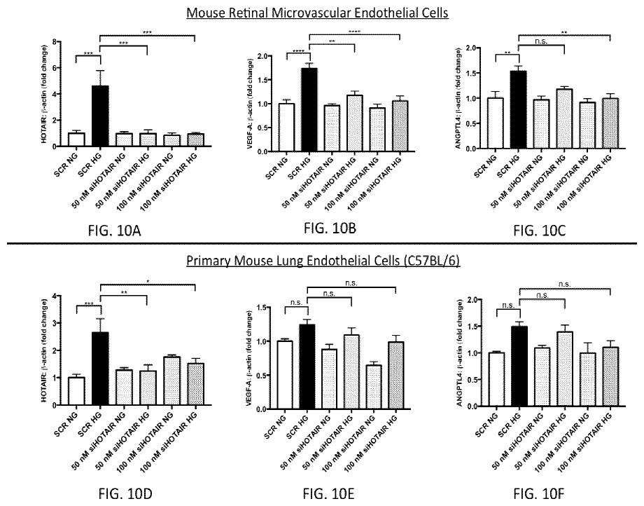

After confirming the significance of Hotair in the retina in diabetes, we sought to evaluate the therapeutic potential for siRNA-mediated modulation of HOTAIR as a new approach to treat DR. In order to determine this, we had first acquired a SMARTpool siHOTAIR that specifically targeted mouse Hotair, tested this siRNA compound on two EC-specific mouse cell lines (mouse retinal microvascular ECs [MRECs] and primary mouse lung ECs [MLECs] (C57/BL6)), and then elucidated the therapeutic significance of siHOTAIR using a short-term, one-month, diabetic animal model. Beginning with our in vitro and ex vivo experiments, we found that 50 nM and 100 nM concentrations of siHOTAIR can evoke significant reductions in Hotair RNA levels across both EC-lines cultured with HG, when compared to SCR HG controls (FIG. 10). In fact, using a 50 nM concentration, ˜79% and ˜53% reductions were observed in MRECs and MLECs challenged with HG, respectively, when compared to SCR HG controls; whereas, at a 100 nM concentration, ˜80% and ˜43% reductions were noted in HG-cultured MRECs and MLECs, respectively (FIGS. 10A and 10D). Similarly, when compared to controls, statistically significant reductions for both Vegf-a and Angptl4 transcripts were also found after Hotair knockdown in MRECs (at a 100 nM concentration; FIGS. 10B and 10C). While, conversely, significant reductions in these angiogenic transcripts were not observed in MLECs (FIGS. 10E and 10F), which suggests that transfection efficiencies may differ between EC subtypes. Nevertheless, based on the findings from MRECs, we selected the mouse-specific siHOTAIR for our subsequent animal experiments.

Intravitreal Administration of siHOTAIR is Non-Toxic and Prevents Early DR-Related Retinal Changes

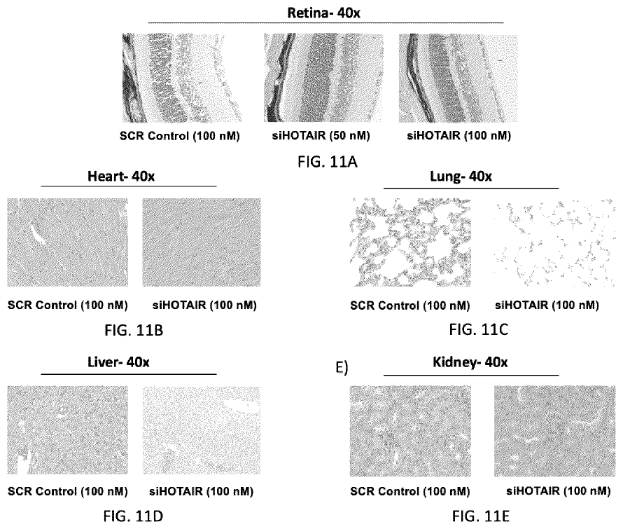

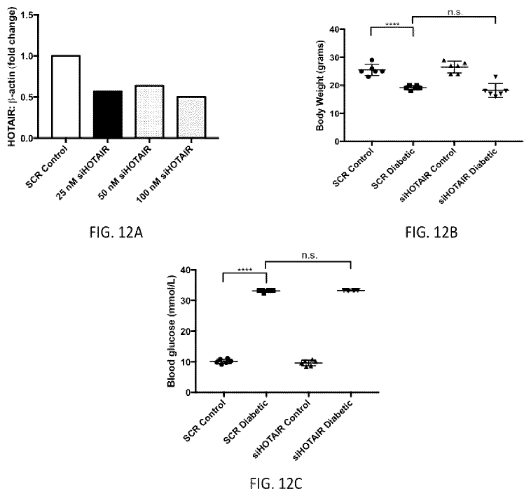

We initially performed a toxicology study involving siHOTAIR. Wild-type C57BL/6 mice were subjected to a one-time intravitreal injection that consisted of either scrambled siRNA control (100 nM; SCR) or siHOTAIR at varying concentrations (25 nM, 50 nM, and 100 nM) and were monitored for seven days and then euthanized for tissue collection. No behavioral changes or ocular complications were observed in the mice throughout the duration of the experiments. As evidenced by hematoxylin and eosin (H&E) staining, no structural abnormalities were observed across retinal, heart, lung, liver, and kidney tissues following intravitreal siHOTAIR injection at 25, 50, or 100 nM concentrations (FIGS. 11A-11E). Furthermore, at the 7-day mark, retinal HOTAIR expressions appeared to be the lowest following a 100 nM dose of siHOTAIR (˜50% reduction) when compared to SCR controls and other siHOTAIR concentrations (FIG. 12A). Using this information, we opted to select 100 nM as the optimal concentration of siHOTAIR for our therapeutic animal model.

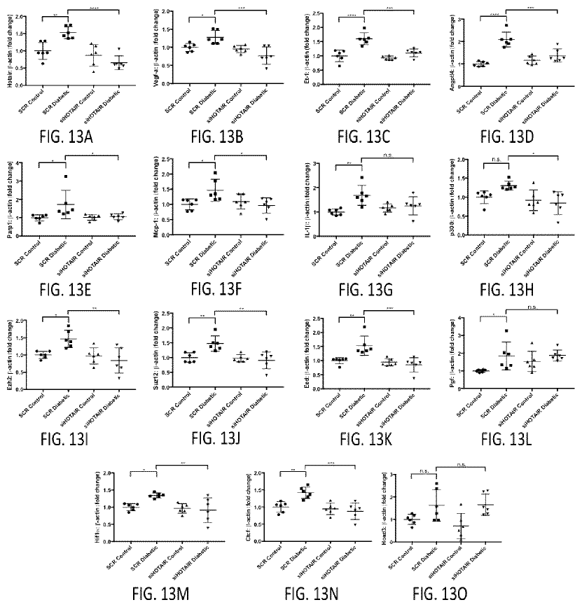

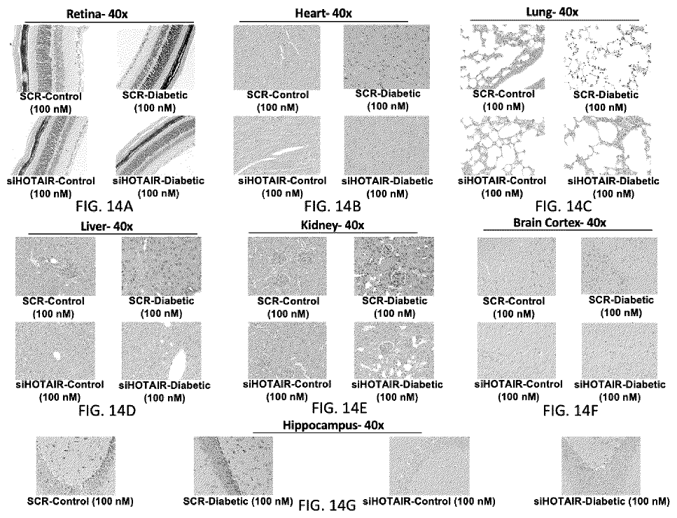

To understand the therapeutic effects of siHOTAIR, diabetes was induced in C57BL/6 mice using STZ injections. All diabetic mice showed significant hyperglycemia and a progressive loss of body weight (FIGS. 12B and 12C), as well as polyuria and glucosuria (data not shown). Compared to SCR diabetic controls, Hotair knockdown did not further affect body weight and blood glucose levels (FIGS. 12B and 12C). When examining the pathogenetic molecules implicated in DR-related microvascular dysfunction, we found elevated RNA expressions of Hotair, Vegf-a, Et-1, Angptl4, Parp, Mcp-1, I1-1f, p300, polycomb repressive complex 2 [Prc2] components (Ezh2, Suz12, and Eed), Pgf, Hif-1α, and Ctcf in the retinal tissues of diabetic mice administered SCR siRNAs (FIGS. 13A-13N). Whereas, the knockdown of Hotair (a ˜58% reduction) could significantly reduce diabetes-induced upregulations of Hotair, Vegf-a, Et-1, Angpl4, Mcp-1, Ctcf, Hif-1α, p300, Prc2 components (Ezh2, Suz12, and Eed), and Parp1—suggesting that HOTAIR knockdown can alleviate early molecular aberrations induced by a diabetic milieu within the retina (FIGS. 13A-13F, 13H-13K and 13M-13N). Of note, although downward trends can be observed, we did not find statistically significant changes in retinal expressions of I1-1β (FIG. 13G), Pgf (FIG. 13L), and Hoxd3 (FIG. 13O) between SCR and siHOTAIR-treated diabetic animals. Furthermore, as indicated by H&E stains and in comparison to SCR controls, we did not find any observable cellular anomalies or toxic effects in retinal, cardiac, lung, liver, kidney, and brain tissues following 1 month of 100 nM siHOTAIR injections (FIGS. 14A-14G).

### HOTAIR is Upregulated in the Vitreous and Serum of PDR Patients

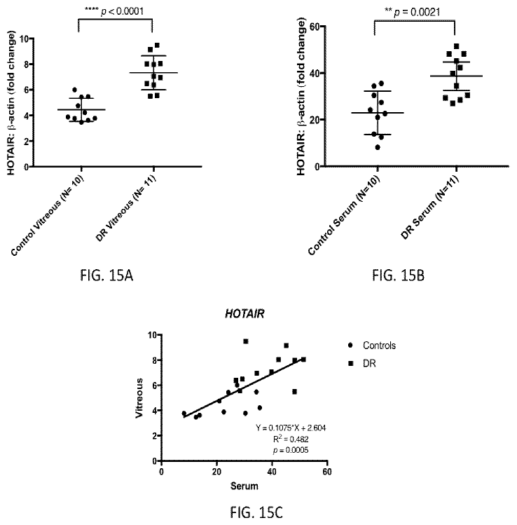

After establishing HOTAIR's biological importance in diabetic animals, we wanted to determine whether HOTAIR expressions have similar clinical importance from a potential biomarker angle. To this extent, we examined HOTAIR expressions in the serum and vitreous humor (VH) of patients with PDR. Based on our RT-qPCR analyses, HOTAIR expressions were distinct and significantly upregulated in the vitreous (p<0.0001; FIG. 15A) and serum (p=0.0021; FIG. 15B) of patients with PDR than that of patients without PDR. Moreover, we performed two-sided Pearson correlations to determine whether a linear association for HOTAIR expressions existed between the two sample types. Interestingly, statistically significant correlations for HOTAIR were found between serum and vitreous samples, where increased serum HOTAIR expressions positively correlated with increased vitreous HOTAIR expressions (p=0.0005, R²=0.482; FIG. 15C). Taken together, our clinical findings suggest that HOTAIR expressions in the vitreous and serum are associated with PDR and can be used as a prognostic and diagnostic biomarker for DR.

### HOTAIR Knockdown can Partially Prevent Glucose-Induced DNA and Mitochondrial Damage, as Well as Disruptions of Endothelial Cell Junctions In Vitro

We then wanted to further explore some of the molecular mechanisms for HOTAIR in vitro. With a previous report documenting HOTAIR's implications in mitochondrial dysfunction in HeLa cells (67) and based on the localization of HOTAIR from our RNA FISH experiments and the impact of siHOTAIR on Cytochrome B RNA levels, we first assessed the mitochondrial transmembrane potential (ΔΨM) in HRECs after HOTAIR knockdown through the detection of JC-1 signals. As shown in FIG. 16A, HG significantly evoked mitochondrial depolarization (indicated by more green and less red fluorescence; low ΔΨM) compared to scrambled NG controls (p<0.0001), suggesting that HG induces mitochondrial depolarization/dysfunction in HRECs. Conversely, when compared to SCR NG controls, the knockdown of HOTAIR in cells cultured with NG can markedly increase mitochondrial activity (indicated by more red and less green fluorescence; normal to high ΔΨM; p<0.0001; FIG. 16B). As expected, HOTAIR knockdown partially reduces HG-induced mitochondrial dysfunction/depolarization when compared to SCR HG controls (p=0.0459; FIG. 16B). Collectively, the JC-1 results indicate that HOTAIR contributes to mitochondrial aberrations in hyperglycemic environments.

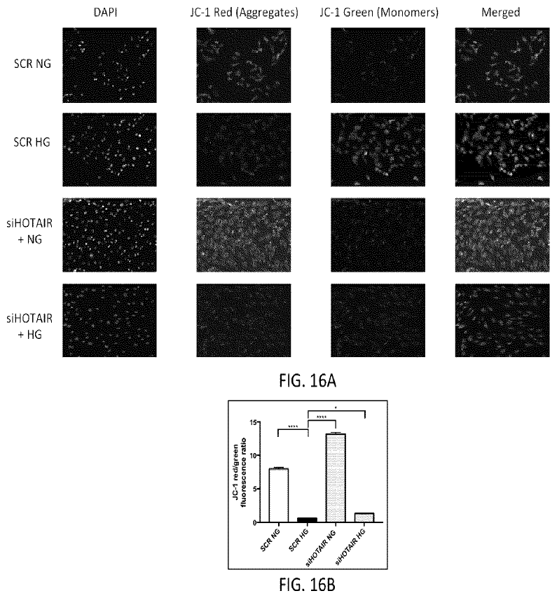

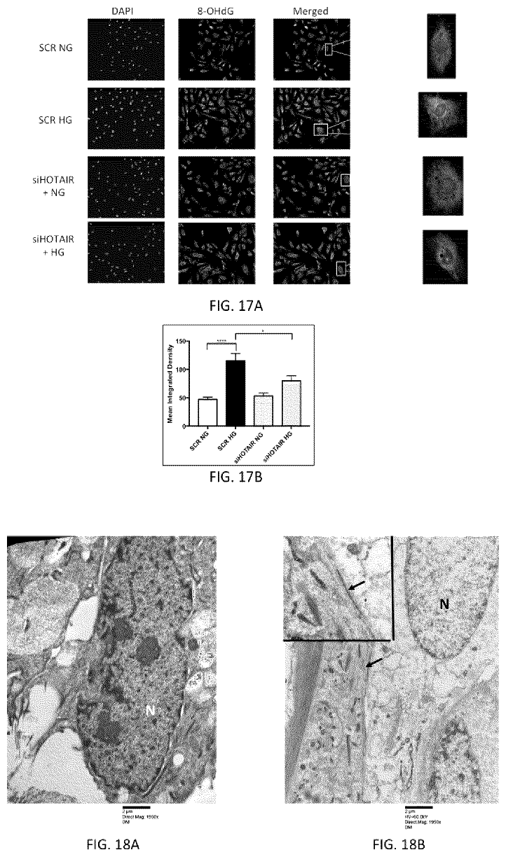

We examined the relationship between HG, HOTAIR, and 8-hydroxy-2′-deoxyguanosine (8-OHdG) levels, a biomarker for oxidative DNA damage. Indeed, in comparison to SCR NG cells, HRECs in the presence of SCR siRNAs and HG demonstrated significant expressions of 8-OHdG (increase in green fluorescence; p<0.0001, FIGS. 17A-17B). In contrast, however, HOTAIR knockdown significantly reduced glucose-induced increases in 8-OHdG expressions compared to SCR HG cells (p=0.0264), which suggests that HOTAIR may be implicated in HG-induced oxidative damage. Moreover, an essential prerequisite in the development of DR is the loss of endothelium, which is caused by chronic hyperglycemic exposure and demonstrated by dysregulated endothelial cell-to-cell junctions (2). To investigate this in our cell culture model, we examined SCR or siHOTAIR-treated HRECs in HG using electron microscopy. HG induced disruptions of cell junctions in HRECs treated with SCR siRNAs (FIG. 18A). However, conversely, the knockdown of HOTAIR preserved EC junctional integrity following HG culture (FIG. 18B). These results further suggest that HOTAIR contributes to DR-related EC dysfunction.

### HOTAIR-Induced Production of DR-Related Molecules Depends on Glycolytic Metabolism

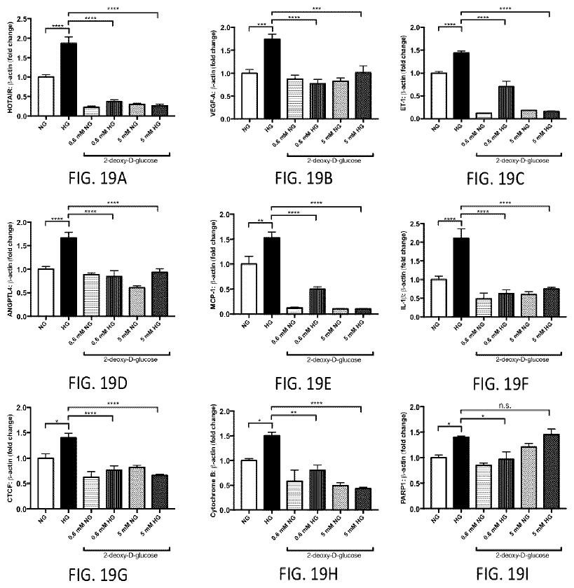

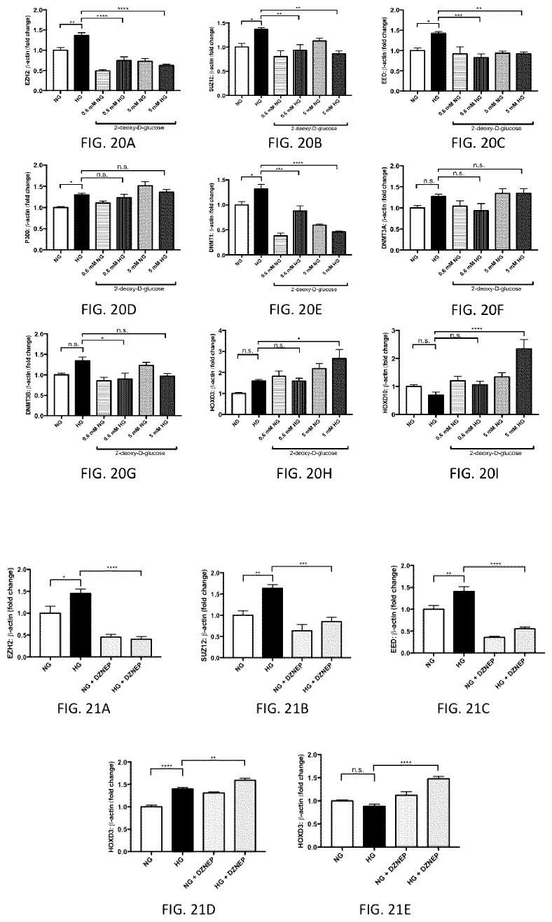

To have a better understanding of the regulatory mechanisms, we sought to examine the upstream role of glucose on HOTAIR and the expression of its target molecules. We employed 2-deoxy-D-glucose (an inhibitor of the glycolytic pathway; 2-DG) and investigated the effects of this glucose analogue on HRECs in vitro. Accordingly, albeit the apoptotic nature of 2-DG (˜45-55% viability indicated by trypan blue exclusion assay [data not shown]), 2-DG treatment significantly blocked HG-induced expressions of HOTAIR, VEGF-A, ET-1, ANGPTL4, MCP-1, IL-1β, CTCF, and Cytochrome B (FIGS. 19A-19H), which further emphasized the upstream regulatory roles played by glucose. Furthermore, inhibiting effective glucose metabolism (at 5 mM of 2-DG) evoked significant reductions in epigenetic molecules including EZH2, SUZ12, EED, and DNMT1 (FIGS. 20A-C and 20E), but no differences were observed for DNMT3A and DNMT3B (FIGS. 20F and 20G). Interestingly, even at 5 mM concentrations, 2-DG treatments also did not induce significant reductions in PARP1 (FIGS. 19I) and P300 (FIG. 20D) expressions and did not augment Cytochrome B expressions, which may suggest that the blockade of glycolysis may continue to produce direct oxidative stress through nuclear transport mechanisms involving PARP1 and P300 rather than contributions of oxidative damage from the mitochondria (68). Moreover, at 5 mM concentrations, HOXD3 and HOXD10 expressions were significantly upregulated in 2-DG-treated HRECs cultured with HG (FIGS. 20H and 20I) compared to HG controls, further highlighting the inverse relationship shared between HOTAIR and HOXD expressions. Taken together and in keeping with previous reports that confirm the anti-angiogenic and apoptotic effects of 2-DG on ECs (58), our data indicates that glucose works upstream of HOTAIR and inhibiting glucose uptake can ultimately prevent the upregulation of HOTAIR and most of its downstream targets.

### Histone Methylation Epigenetically Regulates HOTAIR and its Downstream Targets

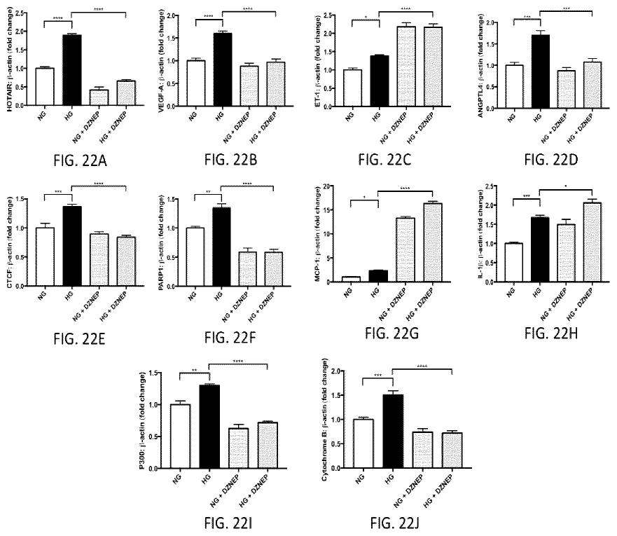

Beginning with the administration of a global histone methylation inhibitor known as 3-deazaneplanocin A (DZNep), we confirmed that HRECs in the presence of HG plus DZNep had significantly reduced expressions of PRC2 components; in particular, EZH2, SUZ12, and EED transcripts were reduced by ˜72% (p<0.0001), ˜48% (p=0.0005), and ˜61% (p<0.0001), respectively, when compared to SCR HG cells (FIG. 21A-C). Accompanying the reduced expressions of PRC2 components in HRECs treated with DZNep and HG, statistically significant reductions were also evident for HOTAIR, VEGF-A, ANGPTL4, CTCF, PARP1, P300, and Cytochrome B transcripts when compared to SCR HG controls (FIGS. 22A-22B, 22D-22F, 22I-22J). On the contrary, opposite trends were observed for ET-1 (FIG. 22C), MCP-1 (FIG. 22G), IL-1β (FIG. 22H), HOXD3 and HOXD10 transcripts, where DZNep pre-treatment plus HG culture of HRECs significantly augmented the expressions of the aforementioned molecules (FIGS. 21D and 21E, and FIG. 22). These dynamic observations are in keeping with our previous studies (27,57) and could suggest that DZNep is not completely selective and as such, may be disrupting a number of different cellular cross-talks in ECs within a hyperglycemic environment.

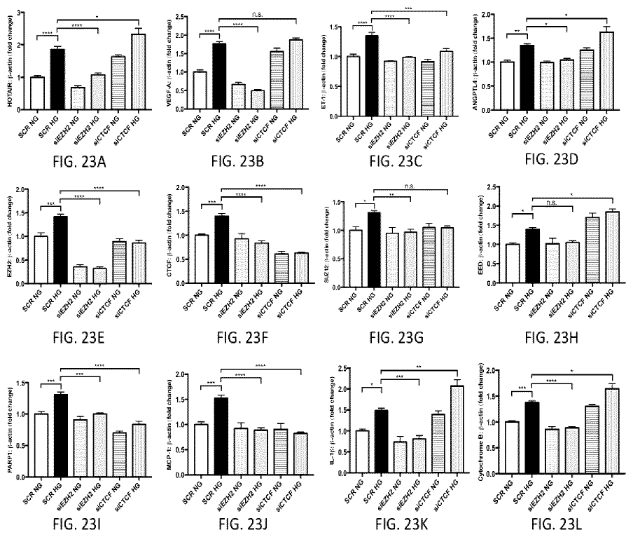

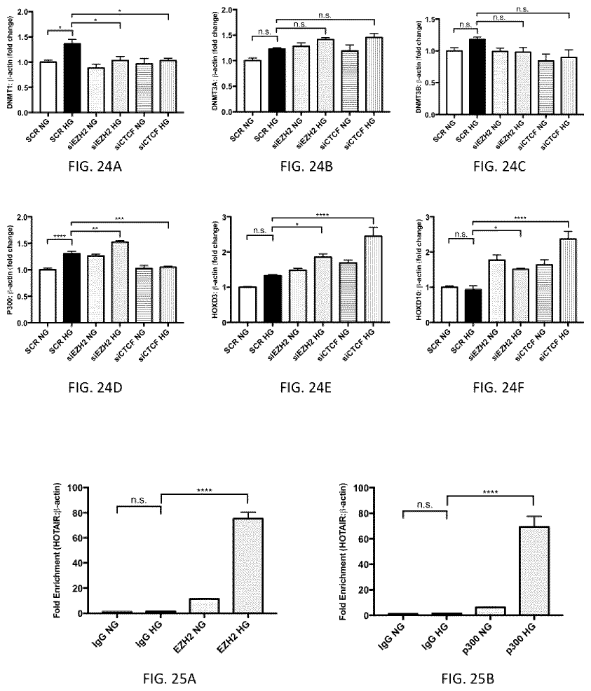

To confirm and expand our experimental findings using DZNep, we selected EZH2 (the catalytic subunit of PRC2 (69)) and CTCF (a critical transcription factor that can maintain chromosome organization and is possibly implicated in the direct regulation of HOTAIR (70,71)) for subsequent siRNA-mediated knockdown. Following siRNA treatments and when compared to SCR HG controls, we confirmed significant reductions for EZH2 (˜77% knockdown; FIG. 23E) and CTCF (˜55% knockdown; FIG. 23F). Interestingly, inhibiting the expressions of EZH2 in HG conditions also evoked significant reductions in HOTAIR, VEGF-A, ET-1, ANGPTL4, CTCF, SUZ12, PARP1, MCP-1, IL-1β, Cytochrome B, and DNMT1 RNA expressions, while significant increases were seen for P300, HOXD3 ad HOXD10 transcript levels, compared to SCR HG controls (FIG. 23 and FIGS. 24A, 24D-F). No significant differences in expressions were observed for DNMT3A and DNMT3B following siEZH2 treatment (FIGS. 24B and 24C). Taken together, these findings imply that EZH2 (the critical component of PRC2) is also directly involved in the transcriptional regulation of HOTAIR and several other downstream genes in a hyperglycemic environment. Of note, the differences observed between ET-1, MCP-1, and IL-1β RNA expressions for DZNep and siEZH2 treatments may have been due to the particular selectivity profile for each compound (i.e., siRNAs are generally more specific in gene knockdown versus global inhibitors for histone methylation).

On the other hand, the knockdown of CTCF in HRECs cultured with HG produced differential expressions of several genes, including significant increases in HOTAIR, ANGPTL4, EED, IL-1β, Cytochrome B, HOXD3, and HOXD10 and significant decreases in ET-1, EZH2, PARP1, MCP-1, DNMT1, and P300 transcripts when compared to their respective SCR HG controls (FIG. 23). As well, no significant differences were observed for VEGF-A, SUZ12, DNMT3A, and DNMT3B transcripts after the silencing of CTCF in HRECs cultured with HG (FIGS. 23 and 24). Based on these results, our collective findings allude to the diverse roles of CTCF in gene regulation, where siRNA-mediated depletion of CTCF can either augment glucose-induced expressions of certain genes (possibly through the inability of CTCF to block the interaction between enhancers and promoters, leading to subsequent gene induction) or repress the expressions of select genes, which may occur due to changes in chromatin architecture that prevent gene induction (71,72).

HOTAIR Binds with Histone Modifying Enzymes and Regulates VEGF Transcription

We then examined for possible direct relationships shared between HOTAIR and critical histone modifying enzymes in HRECs and thus, we performed a RNA immunoprecipitation (RIP). In comparison to IgG controls, our results demonstrated that HOTAIR RNA levels were distinctly enriched in the precipitated anti-EZH2 and P300-antibody fractions obtained from HRECs cultured in HG (p<0.0001; FIG. 25), suggesting that HG promotes strong HOTAIR binding associations to EZH2 and P300. Our findings are in agreement with previous reports that have documented similar relationships with HOTAIR and these epigenetic mediators (19,73).

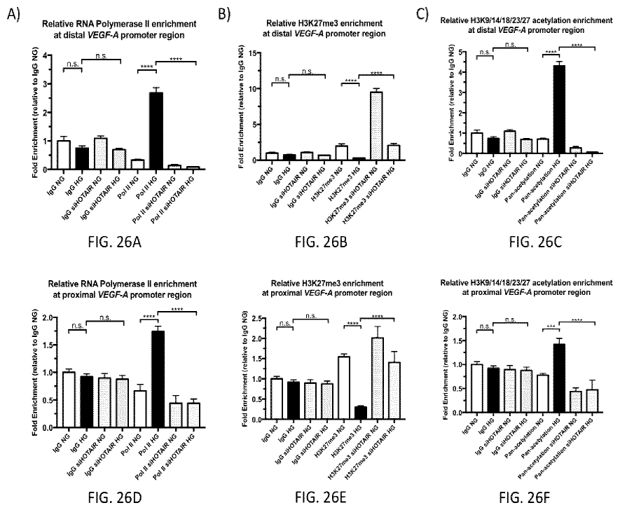

Next, to demonstrate the involvement of histone modifications at the genomic level, we performed chromatin immunoprecipitation (ChIP)-qPCR using antibodies for IgG (negative control), RNA polymerase II (indicative of transcriptional activity; Pol II), H3K27me3 (indicative of transcriptional repression), and pan-H3K9/14/18/23/27 acetylation (indicative of transcriptional activation). We treated HRECs with siHOTAIR and employed primers that specifically spanned across the proximal and distal promoter regions of VEGF-A for subsequent ChIP-qPCR analyses. Accordingly, compared to NG controls, RNA Pol II levels were significantly enriched in both the distal (p<0.0001; FIG. 26A) and proximal promoter (p<0.0001; FIG. 26D) regions of VEGF-A in HRECs cultured with HG; whereas, the knockdown of HOTAIR can markedly reduce Pol II enrichment in these regions compared to HG controls. Conversely, under HG stimulation, significant reductions of H3K27me3 enrichment were observed in both VEGF-A distal (p<0.0001; FIG. 26B) and proximal promoter (p<0.0001; FIG. 26E) regions and siHOTAIR treatment significantly reversed glucose-induced reductions of H3K27me3 in the VEGF-A promoter. Moreover, when compared to NG controls, HG conditions significantly augmented the enrichment of H3K9/14/18/23/27 acetylation in both VEGF-A promoter regions, while the knockdown of HOTAIR significantly prevented glucose-induced increases in pan-acetylation levels of H3K9/14/18/23/27 across the VEGF-A distal (p<0.0001; FIG. 26C) and proximal promoter (p<0.0001; FIG. 26F) regions, compared to HG groups. Hence, we concluded that a dynamic interplay exists between HOTAIR, histone-modifying enzymes, and RNA Pol II in the transcriptional regulation of genes, such that HOTAIR may have an active role in modulating the epigenome during hyperglycemic stress. Of note, no significant differences were observed between IgG NG and HG groups, confirming the specificity of the antibodies.

Duration-Dependent and Glucose-Induced Alterations of CpG Methylation Patterns Across the HOTAIR Gene were not Observed in HRECs

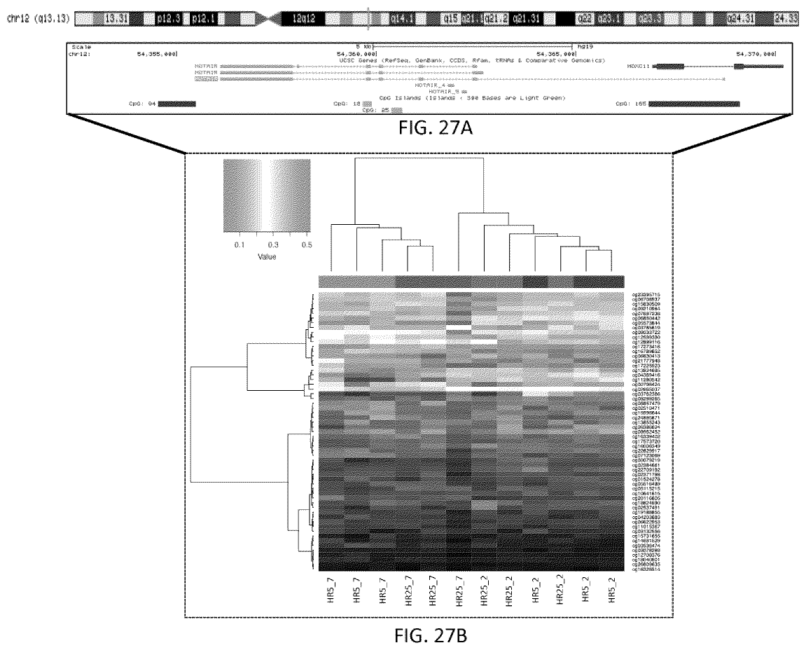

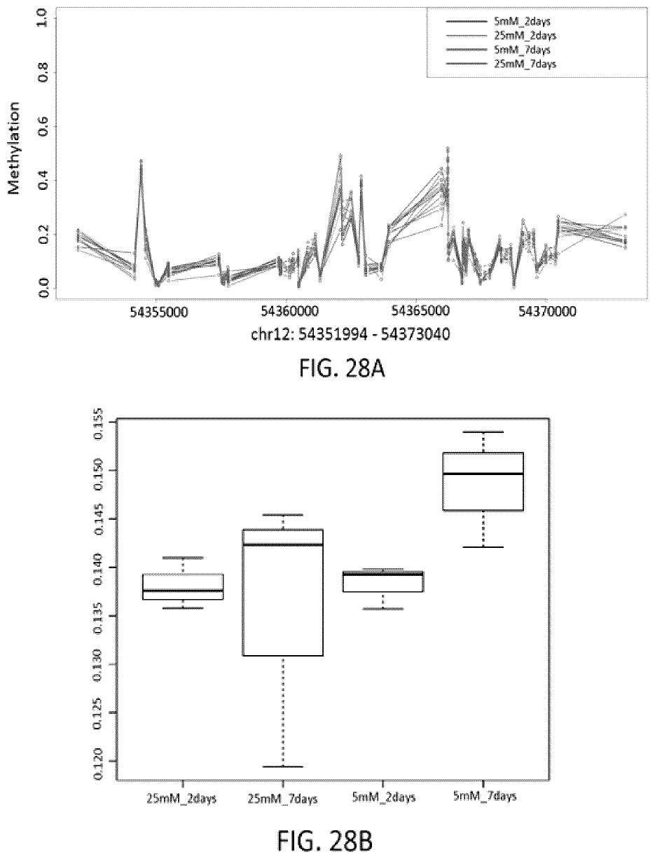

In order to investigate glucose-induced implications of DNA methylation on HOTAIR regulation, we incubated HRECs in NG and HG conditions for durations of 2 and 7 days and then performed a genome-wide DNA methylation experiment using Infinium EPIC arrays and quality controls. Following the detection of >860,000 CpG sites (probes), we exclusively selected CpG sites that spanned across the HOTAIR gene (5 kb upstream to 1 kb downstream of the gene), which corresponded to 59 probes (FIG. 27). We found that the average methylation intensity was generally lower for a majority of the probes (β-values<0.3), except for 7 probes where slightly greater methylation intensities were observed (0.2<β-values<0.5; these sites mainly corresponded to North/South Shelf and North/South Shore regions; FIG. 27). Furthermore, when examining the methylation patterns between the various groups across the HOTAIR genomic region (chromosome 12: 54,351,994 to 54,373,040; FIG. 28A), it was interesting to observe that a stable DNA methylation pattern persisted across all groups despite different culture durations (2 and 7 days) and glucose concentrations (NG and HG). However, of note, although not statistically significant, HRECs stimulated with HG at both 2 and 7 days displayed a slight trend towards the reduction of DNA methylation intensities in the HOTAIR promoter, compared to their respective NG controls (FIG. 28B). Nevertheless, these findings may allude to the stable epigenetic nature of DNA methylation marks in HRECs during hyperglycemic stress (61).

Blockade of DNA Methyltransferases Differentially Regulates the Expressions of HOTAIR and some of its targets

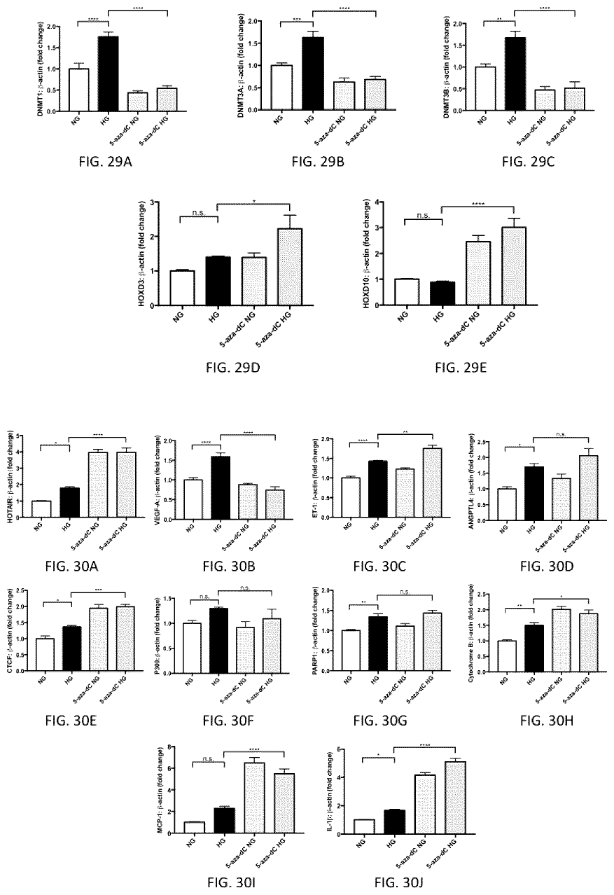

We then wanted to examine the cause-effect relationship of genome-wide DNA methylation on the expressions of HOTAIR and its downstream targets. Accordingly, we pre-treated HRECs with the DNA de-methylating agent, 5-Aza-2′-deoxycytidine (5-aza-dC) prior to NG or HG culture. Following 5-aza-dC administration and compared to HG controls, DNMTJ, DNMT3A, and DNMT3B RNA levels were reduced by ˜69%, ˜58%, and ˜69%, respectively (p<0.0001; FIGS. 29A-C). Accompanying the significantly reduced expressions of DNMTs in 5-aza-dC-treated HRECs, we also observed significant elevations in HOTAIR, ET-1, CTCF, Cytochrome B, MCP-1, IL-1β, HOXD3, and HOXD10 transcripts, while no significant differences were observed for ANGPTL4, P300, and PARP1 expressions (FIGS. 29D and 29E, FIG. 30). Intriguingly, however, globally inhibiting the expressions of DNMTs significantly prevented glucose-induced increases in VEGF-A RNA expressions (p<0.0001; FIG. 30B), which is in keeping with previous observations documented by others (74,75).

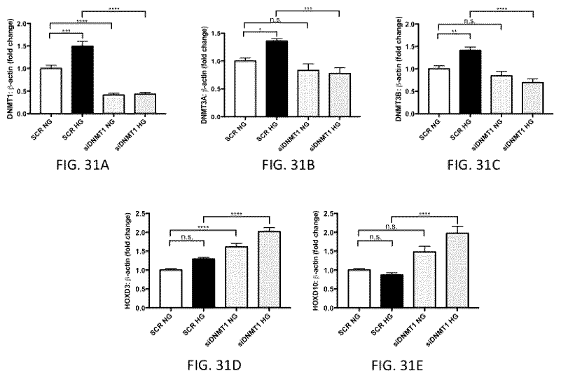

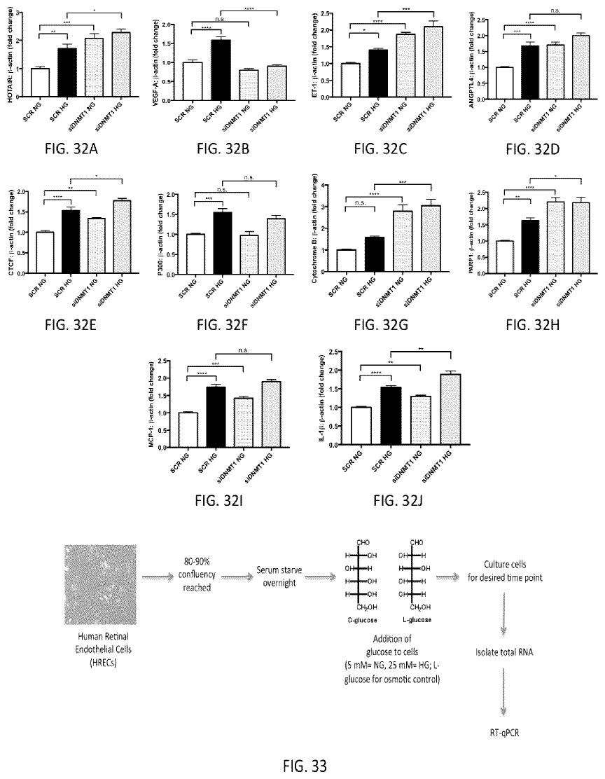

To further confirm the findings from our 5-aza-dC experiments, we specifically silenced DNMT1 (a constitutively expressed DNMT) using a siRNA-mediated approach. With a ˜71% knockdown in DNMT1 RNA levels following the administration of siDNMT1 and HG (p<0.0001; FIG. 31A), DNMT3A and DNMT3B also exhibited significant reductions in transcript expressions by ˜43% (p=0.0005; FIG. 31B) and ˜51% (p<0.0001; FIG. 31C), respectively. In parallel, significant increases in RNA expressions were observed for HOTAIR, ET-1, CTCF, Cytochrome B, PARP1, IL-1β, HOXD3, and HOXD10 after the knockdown of DNMT1 in HG-cultured cells, relative to SCR HG controls (FIGS. 31D and 31E, FIG. 32). Although no significant differences were observed for ANGPTL4, P300, and MCP-1 transcripts after knockdown in HG conditions, significant reductions in VEGF-A transcripts still remained in siDNMT1-treated HRECs cultured with HG (p<0.0001; FIG. 32B), confirming the observations from our 5-aza-dC experiments. Indeed, it may be possible that depending on the genomic location, the inhibition of DNA methylation can have varying methylating effects on distal or intragenic regulatory elements with different degrees of CpG density, which subsequently dictate the regulation of gene expression (76). Nevertheless, our findings suggest that DNA methylation is critically implicated in the regulation of HOTAIR and its target molecules in hyperglycemic environments.

TABLE 1
qPCR primers for all human-specific genes

| Target | | |
|---|---|---|
| Gene (Human): | Oligonucleotide Sequence (5′→3′): | SEQ ID: |
| ACTB | F: CCTCTATGCCAACACAGTGC | 1 |
| | R: CATCGTACTCCTGCTTGCTG | 2 |
| HOTAIR | F: GGTAGAAAAAGCAACCACGAAGC | 3 |
| | R: ACATAAACCTCTGTCTGTGAGTGCC | 4 |
| VEGFA | F: GAACTTTCTGCTGTCTTGGG | 5 |
| | R: CTTCGTGATGATTCTGCCCT | 6 |
| EDN1 | F: AAGCCCTCCAGAGAGCGTTAT | 7 |
| | R: CCGAAGGTCTGTCTGTCACCAATGT | 8 |
| ANGPTL4 | F: GGACACGGCCTATAGCCTG | 9 |
| | R: CTCTTGGCGCAGTTCTTGTC | 10 |
| PGF | F: CCGGCTCGTGTATTTATTACCG | 11 |
| | R: GGCAACCACTGTTCTCCAGAGC | 12 |
| IL1ß | F: GCGGCATCCAGCTACGAATCT | 13 |
| | R: GGGCAGGGAACCAGCATCTT | 14 |
| HIF1α | F: CATAAAGTCTGCAACATGGAAGGT | 15 |
| | R: ATTTGATGGGTGAGGAATGGGTT | 16 |
| PARP1 | F: CCACACACAATGCGTATGAC | 17 |
| | R: CCACAGCAATCTTCGGTTATG | 18 |
| CYTB | F: TCACCAGACGCCTCAACCGC | 19 |
| | R: GCCTCGCCCGATGTGTAGGA | 20 |
| MCP1 | F: TCGCCTCCAGCATGAAAGTC | 21 |
| | R: GGCATTGATTGCATCTGGC | 22 |
| CTCF | F: GACCCCACCCTTCTTCAGATG | 23 |
| | R: CCACAGCAGCCTCTGCTTCT | 24 |
| P300 | F: GGGACTAACCAATGGTGGTG | 25 |
| | R: ATTGGGAGAAGTCAAGCCTG | 26 |
| EZH2 | F: CCACCATTAATGTGCTGGAA | 27 |
| | R: TTCCTTGGAGGAGTATCCACA | 28 |
| SUZ12 | F: TACGGCTCCTATTGCCAAAC | 29 |
| | R: TGCTTCAGTTTGTTGCCTTG | 30 |
| EED | F: GCAACTGTAGGAAGCAACAGA | 31 |
| | R: CATAGGTCCATGCACAAGTGT | 32 |
| DNMT1 | F: ACGGTGCTCATGCTTACAAC | 33 |
| | R: TTAGCCTCTCCATCGGACTT | 34 |
| DNMT3A | F: GGCAAATTCTCAGTGGTGTG | 35 |
| | R: GTCACTCTCATCGCTGCTGT | 36 |
| DNMT3B | F: TTGAATATGAAGCCCCCAAG | 37 |
| | R: TGATATTCCCCTCGTGCTTC | 38 |
| HOXD3 | F: CAGCCTCCTGGTCTGAACTC | 39 |
| | R: ATCCAGGGGAAGATCTGCTT | 40 |
| HOXD10 | F: ATGTACATGCCACCACCTAGC | 41 |
| | R: TTGCTGTGTAACAGGTTGCTC | 42 |

TABLE 2
qPCR primers for all mouse-specific genes.

| Target Gene | Oligonucleotide | SEQ |
|---|---|---|
| (Mouse): | Sequence (5′→3′): | ID: |
| Actb | F: CCTCTATGCCAACACAGTGC | 1 |
| | R: CATCGTACTCCTGCTTGCTG | 2 |
| Hotair | F: GCGCCAACGTAGACCAAAAG | 43 |
| | R: TCTACCGATGTTGGGGACCT | 44 |
| Vegfa | F: ATGCGGATCAAACCTCACCA | 45 |
| | R: CTTTCTTTGGTCTGCATTCAC | 46 |
| Edn1 | F: TTAGCAAGACCATCTGTGTG | 47 |
| | R: GAGTTTCTCCCTGAAATGTG | 48 |
| Angptl4 | F: TTGGTACCTGTAGCCATTCC | 49 |
| | R: GAGGCTAAGAGGCTGCTGTA | 50 |
| Pgf | F: TGCTGGGAACAACTCAACAG | 51 |
| | R: CCTCATCAGGGTATTCATCCA | 52 |
| IL1ß | F: TTCAGGCAGGCAGTATCACTC | 53 |
| | R: GAAGGTCCACGGGAAAGACAC | 54 |
| Hif1α | F: TCAAGTCAGCAACGTGGAAG | 55 |
| | R: TATCGAGGCTGTGTCGACTG | 56 |
| Parp1 | F: GGAAAGGGATCTACTTTGCCG | 57 |
| | R: TCGGGTCTCCCTGAGATGTG | 58 |
| Cytb | F: TCCTTCATGTCGGACGAGGC | 59 |
| | R: AATGCTGTGGCTATGACTGCG | 60 |
| Mcp1 | F: TTGTCACCAAGCTCAAGAGAGA | 61 |
| | R: GAGGTGGTTGTGGAAAAGGTAG | 62 |
| Ctcf | F: TGGTCCAGATGGCGTAGAGG | 63 |
| | R: GTCATCGAGATCCGGCTCAG | 64 |
| p300 | F: AGGCAGAGTAGGACAGTGAA | 65 |
| | R: CTCAGTCTGGGTCACTCAAT | 66 |
| Ezh2 | F: CGCGGGACTAGGGAGTGTTCAGT | 67 |
| | R: AGTACATTATAGGCACCGAGGCGA | 68 |
| Suz12 | F: AGCTCTGCCACAGCAGGTTCA | 69 |
| | R: TGCTTTTGTTCTTTTTGGCCTGCAA | 70 |
| Eed | F: ATGCTGTCAGTATTGAGAGTGGC | 71 |
| | R: GAGGCTGTTCACACATTTGAAAG | 72 |
| Hoxd3 | F: GAGACCTGGCACTGGGAATA | 73 |
| | R: TCCAGGGGAAGATCTGTTTG | 74 |
| Hoxd10 | F: ATAAGCGCAACAAACTCATTTCG | 75 |
| | R: ATATCGAGGGACGGGAACCT | 76 |

TABLE 3
Specific microarray readout for HOTAIR.

| ProbeName | ASHGA5P015895 |
|---|---|
| Absolute Fold change ([HG1] | 2.6724913 |
| vs [NG1]) | |
| Regulation and Type | Up; non-coding |
| Seqname | NR_047517 |
| Gene Symbol | HOTAIR |
| RNA length | 2370 |
| chrom | 12 |
| Sequence | GAAGGAAAGCCCTCCAGCCTCCAGGCC |
| | CTGCCTTCTGCCTGCACATTCTGCCCTG |
| | ATTTC (SEQ ID NO: 121) |

### Example 2

Disclosed is a novel lncRNA-based panel to accurately monitor the progression of diabetic complications. Presently, there are no established lncRNA-based diagnostic panels used for diabetes and its complications. Patients oftentimes have difficulty accessing ocular specialists who possess multimodal retinal imaging tools and so, with the panel presented herein, examining these markers in serum of a patient will be a lot more efficient for patients to access (these markers can be analyzed when patients provide their annual blood work).

### Methods

### Clinical Sample Collection

The Western Research Ethics Board and Lawson Health Research Institute at the University of Western Ontario (London, ON, CAN) approved the clinical component of this study. Patients provided informed consent prior to the procurement of specimens and all of the samples were handled in accordance with the Declaration of Helsinki. Both serum and undiluted vitreous humor (VH) were collected from patients undergoing a pars plana vitrectomy by an experienced vitreoretinal surgeon. Both specimens were categorized into two groups: control and proliferative diabetic retinopathy (PDR). The PDR group comprised of patients diagnosed with advanced stages of DR (PDR; n=11; mean age ±SD=60.7±10.72 years; 10 males and 1 female), while the control group consisted of patients that had no previous history of PDR and were diagnosed with idiopathic macular hole or a separate non-diabetic ocular condition (n=10; mean age ±SD=69.2±8.87 years; 2 males and 8 females). PDR was defined as the presence of neovascularization or fibrous proliferation of the disc or elsewhere on the retina. As previously described (27,56), total RNA was extracted from 500 μL of VH samples and 200 μL of serum samples using the TRIzol reagent (Invitrogen) and a serum RNA extraction kit (Bio Basic Inc., Markham, ON, CAN) following the manufacturer's protocol. After conversion to cDNA, a lncRNA PCR panel, involving RT-qPCR approaches, was used to evaluate the expressions of 9 distinct lncRNAs in these samples.

### Development of the Multi-lncRNA PCR Panel

Customized human lncRNA primers were developed (Table 4) and subsequently aliquoted and lyophilized into a 96-well plastic qPCR plate. Following lyophilization, custom qPCR plates were stored in −20° C. prior to use. A single panel (consisting of 10 wells) in the lncRNA PCR plate examines 9 distinct lncRNAs (MALAT1, HOTAIR, H19, MEG3, ANRIL, MIAT, WISPER, ZFAS1, and HULC) and one house-keeping gene (β-actin). Synthesized cDNA (following the aforementioned RNA isolation and RT-qPCR protocol) was diluted and combined with SYBR-green master mix, and then aliquoted into the 96-well PCR plate containing the pre-aliquoted PCR panel. The panel was then inserted into the LightCycler 96 System for amplification.

### Results

HG-Cultured HRECs Demonstrate Differential Expressions of lncRNAs at 48 Hours.

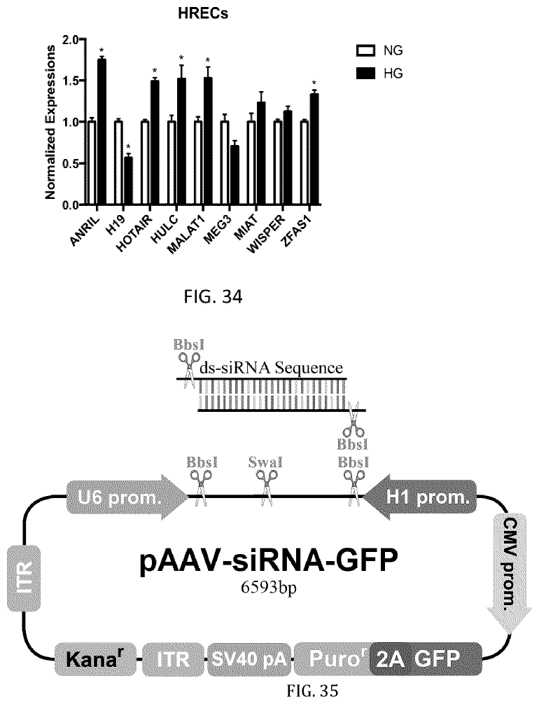

Using our highly established cell culture model (FIG. 33) and RT-qPCR, we confirmed the lncRNA expressions of ANRIL, H19, HOTAIR, HULC, MALAT1, MEG3, MIAT, WISPER, and ZFAS1 in HRECs exposed to 25 mM (HG) or 5 mM (NG) glucose over 48 hours from our array (FIG. 34).

We additionally wanted to determine whether the selected lncRNAs could be detected in human DR. So, we acquired both vitreous humor and serum samples from non-diabetic and diabetic patients undergoing vitrectomy (through collaboration with an ophthalmologist) and directly measured the RNA levels of HOTAIR, MALAT1, H19, WISPER, ZFAS1, HULC, MEG3, MIAT, and ANRIL using our customized qPCR-based panel. Similar to the trends observed in our endothelial cells, we found differential expressions of lncRNAs in the vitreous and serum of patients with PDR compared to non-PDR patients—further confirming the biological relevance of lncRNAs at the human level.

Differential Expressions of lncRNAs in the Serum of Patients.

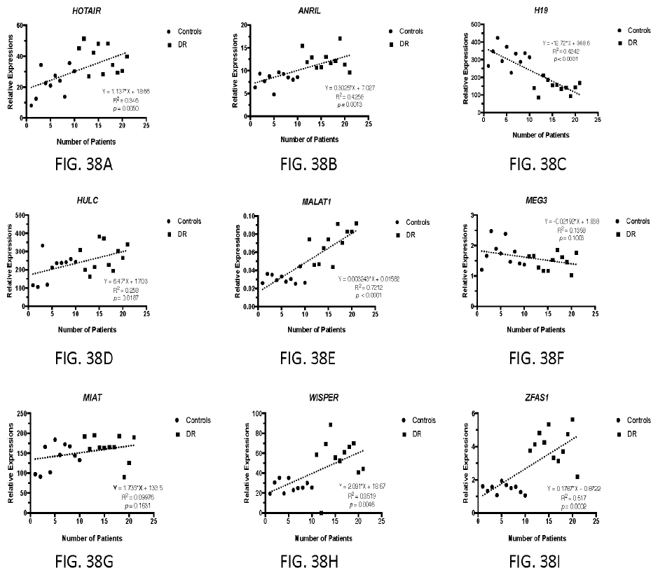

As shown in FIG. 38, there are significant relationships between PDR and serum expressions of HOTAIR (FIG. 38A), ANRIL (FIG. 38B), H19 (FIG. 38C), HULC (FIG. 38D), MALAT1 (FIG. 38E), WISPER (FIG. 38H) and ZFAS1 (FIG. 38I). Although we did not find a significant relationship between PDR and serum expressions of MEG3 (FIG. 38F) or MIAT (FIG. 38G), we anticipate that an increase in sample size may help improve the significance of this relationship.

Differential Expressions of lncRNAs in the Vitreous Fluid of Patients.

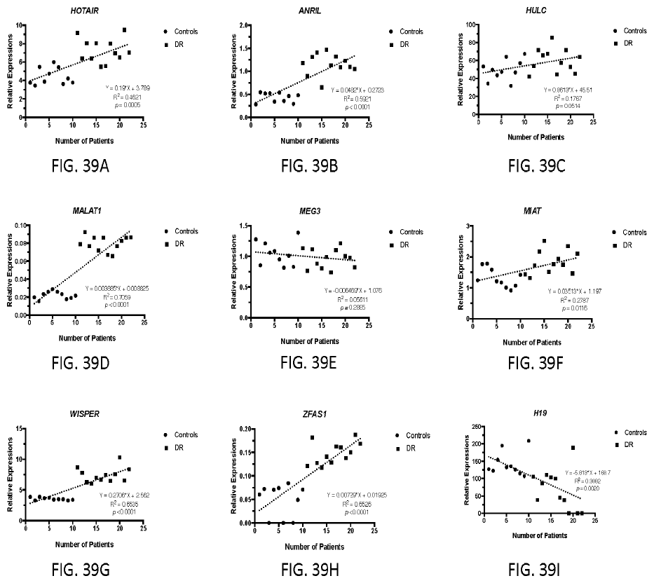

As shown in FIG. 39, there is a significant relationship between PDR and the vitreous expressions of HOTAIR (FIG. 39A), ANRIL (FIG. 39B), MALAT1 (FIG. 39D), MIAT (FIG. 39F), WISPER (FIG. 39G), ZFAS1 (FIG. 39H) and H19 (FIG. 39I). Although we did not find a significant relationship between PDR and HULC (FIG. 39C) or MEG3 (FIG. 39E), we anticipate that an increase in sample size may help improve the significance of this relationship.

### Pearson Correlation Analyses Between Serum and Vitreous Samples.

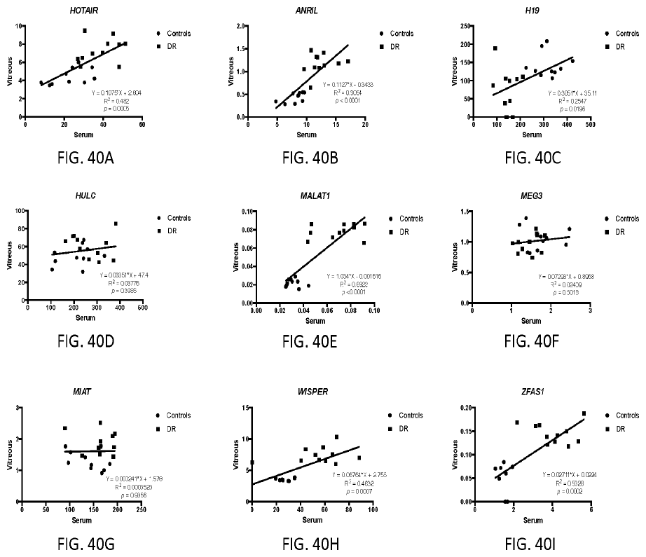

When comparing between serum and vitreous samples, significant correlations were observed for HOTAIR (FIG. 40A), ANRIL (FIG. 40B), H19 (FIG. 40C), MALAT1 (FIG. 40E), WISPER (FIG. 40H) and ZFAS1 (FIG. 40I), which suggests that the expressions of these lncRNAs can be reflected from the serum and vitreous of patients with PDR (FIG. 40). Although we did not find significant correlations between serum and vitreous concentrations of HULC (FIG. 40D), MEG3 (FIG. 40F), and MIAT (FIG. 40G), including a larger sample size may further help confirm the relationships between these markers and sample types.

TABLE 4
Human lncRNA qPCR primers used for the lncRNA
array.

| Target Gene | Oligonucleotide | SEQ |
|---|---|---|
| (Human): | Sequence (5′→3′): | ID: |
| ACTB | F: CCTCTATGCCAACACAGTGC | 1 |
| (B-actin) | R: CATCGTACTCCTGCTTGCTG | 2 |
| HOTAIR | F: GGGGCTTCCTTGCTCTTCTTATC | 77 |
| | R: CTGACACTGAACGGACTCTGTTTG | 78 |
| H19 | F: AAAGACACCATCGGAACAGC | 79 |
| | R: AGAGTCGTCGAGGCTTTGAA | 80 |
| WISPER | F: CCATCTGTGGGACATCTGTG | 81 |
| | R: TGGGGGCTGTGGAGATAGTA | 82 |
| ZNFX1-AS1 | F: CAGCGGGTACAGAATGGA | 83 |
| (ZFAS1) | R: TCAGGAGATCGAAGGTTGTAGA | 84 |
| HULC | F: ATCTGCAAGCCAGGAAGAGTC | 85 |
| | R: CTTGCTTGATGCTTTGGTCTGT | 86 |
| ANRIL | F: CCCTTATTTATTCCTGGCTCC | 87 |
| | R: GACCTCGCTTTCCTTTCTTCC | 88 |
| MALAT1 | F: TCTTAGAGGGTGGGCTTTTGTT | 89 |
| | R: CTGCATCTAGGCCATCATACTG | 90 |
| MIAT | F: GGGAGGGGAAATGGGTGATGTA | 91 |
| | R: TAACGCCAAATGTGAAGTGTGA | 92 |
| MEG3 | F: CGGCTGGGTCGGCTGAAGAACT | 93 |
| | R: CCGCCCAAACCAGGAAGGAGAC | 94 |

TABLE 5
Serum analysis of non-diabetic (C) and diabetic patients with proliferative diabetic retinopathy (P). Sensitivity and
specificity of individual analytes were determined with an arbitrary cut-off value. As demonstrated by these
data, this approach may be used as a relatively non-invasive approach for the screening of
diabetic retinopathy and other possible diabetes-related end-organ damage.

| Sample | ANRIL | H19 | HOTAIR | HULC | MALAT1 | MEG3 | MIAT | WISPER | ZFAS1 |
|---|---|---|---|---|---|---|---|---|---|
| C1 | 6.317035 | 264.3433 | 8.14002 | 115.997 | 0.02588 | 1.20596 | 97.1368 | 19.4906 | 1.60034 |
| C2 | 9.344832 | 347.3061 | 12.4677 | 105.307 | 0.03605 | 1.65872 | 90.9377 | 30.4493 | 1.33210 |
| C3 | 7.688176 | 423.0924 | 34.3501 | 332.628 | 0.0351 | 2.47466 | 165.946 | 35.2841 | 1.56118 |
| C4 | 8.764056 | 292.1339 | 22.5636 | 118.645 | 0.02914 | 1.89127 | 101.926 | 19.5273 | 1.06095 |
| C5 | 4.765568 | 372.5143 | 20.9521 | 211.749 | 0.0331 | 1.74201 | 184.091 | 35.2548 | 1.93827 |
| C6 | 9.632179 | 224.8733 | 27.3805 | 237.013 | 0.02755 | 2.38225 | 145.521 | 22.9589 | 1.67749 |
| C7 | 9.311572 | 333.5591 | 24.1946 | 238.121 | 0.03042 | 1.46032 | 172.123 | 24.8837 | 1.49828 |
| C8 | 8.526132 | 287.6585 | 13.7985 | 241.517 | 0.02515 | 1.79963 | 167.368 | 25.4144 | 1.57615 |
| C9 | 8.024799 | 336.5828 | 35.5420 | 258.842 | 0.04438 | 1.41119 | 144.348 | 29.9976 | 1.25427 |
| C10 | 8.599776 | 313.5936 | 30.3786 | 242.753 | 0.02628 | 1.36861 | 132.602 | 25.4255 | 1.05221 |
| P1 | 15.45679 | 137.6733 | 45.1626 | 308.462 | 0.07408 | 1.63966 | 191.527 | 58.6126 | 3.75264 |
| P2 | 11.88811 | 84.3913 | 51.4208 | 201.177 | 0.04586 | 1.65425 | 160.431 | 0 | 4.13490 |
| P3 | 12.89566 | 210.7196 | 26.9624 | 162.753 | 0.04681 | 1.27289 | 194.468 | 69.0886 | 4.82809 |
| P4 | 10.64712 | 185.3472 | 42.3449 | 215.395 | 0.06475 | 1.16321 | 164.306 | 88.6474 | 4.24692 |
| P5 | 10.76938 | 154.3911 | 48.1498 | 381.835 | 0.07424 | 1.16991 | 163.418 | 55.5047 | 5.34691 |
| P6 | 13.02459 | 155.8962 | 28.3948 | 371.854 | 0.04373 | 1.52657 | 165.138 | 52.3387 | 3.32712 |
| P7 | 11.64394 | 134.7698 | 48.2044 | 225.712 | 0.09124 | 1.85019 | 164.667 | 60.7757 | 3.13538 |
| P8 | 12.12767 | 139.6844 | 34.4741 | 194.549 | 0.07034 | 1.61837 | 192.794 | 66.5354 | 3.71290 |
| P9 | 17.07384 | 93.25907 | 29.2784 | 302.516 | 0.08282 | 1.45534 | 90.0150 | 69.9743 | 4.74009 |
| P10 | 11.36953 | 142.3843 | 30.5124 | 264.669 | 0.08270 | 1.02424 | 125.930 | 40.5173 | 5.63456 |
| P11 | 9.577154 | 167.516 | 39.7824 | 339.653 | 0.09179 | 1.76496 | 189.868 | 44.263 | 2.17985 |
| Sensitivity | 100 | 90.9 | 64 | | 100 | | | 91 | 100 |
| Specificity | 77 | 100 | 100 | | 80 | | | 100 | very high |
| PPV | 100 | 100 | 100 | | 86 | | | 100 | 100 |
| NPV | 100 | 91 | 78 | | 100 | | | 91 | 100 |
| Cut-off | >9 | <200 | >36 | | >0.035 | | | >37 | >2 |

TABLE 6
Detailed information regarding the molecular weights and sequences for the siRNAs
targeting Human HOTAIR (top) and mouse Hotair (bottom) in the SMARTpool solutions
(commercially available) used in our experiments.
R-187951-00-0005, Lincode Human HOTAIR (100124700) siRNA - SMARTpool, 5 nmol

| | |
|---|---|
| Lincode SMARTpool siRNA N-187951-01, | Lincode SMARTpool siRNA N-187951-02, |
| HOTAIR | HOTAIR |
| Mol. Wt. 13,429.8 (g/mol) | Mol. Wt. 13,414.9 (g/mol) |
| Ext. Coeff. 369,884 (L/mol·cm) | Ext. Coeff. 377,627 (L/mol·cm) |
| Target Sequence: | Target Sequence: |
| AGACGAAGGUGAAAGCGAA | CAAUAUAUCUGUUGGGCGU |
| (SEQ ID: 95) | (SEQ ID: 96) |
| Lincode SMARTpool siRNA N-187951-03, | Lincode SMARTpool siRNA N-187951-04, |
| HOTAIR | HOTAIR |
| Mol. Wt. 13,474.8 (g/mol) | Mol. Wt. 13,429.9 (g/mol) |
| Ext. Coeff. 356,890 (L/mol·cm) | Ext. Coeff. 371,397 (L/mol·cm) |
| Target Sequence: | Target Sequence: |
| GGGACUGGGAGGCGCUAAU | CAGUGGAAUGGAACGGAUU |
| (SEQ ID: 97) | (SEQ ID: 98) |

R-173526-00-0005, Lincode Mouse Hotair (100503872) siRNA - SMARTpool, 5 nmol

| | |
|---|---|
| Lincode SMARTpool siRNA N-173526-01, | Lincode SMARTpool siRNA N-173526-02, |
| Gm16258 | Gm16258 |
| Mol. Wt. 13,399.8 (g/mol) | Mol. Wt. 13,444.8 (g/mol) |
| Ext. Coeff. 380,030 (L/mol·cm) | Ext. Coeff. 361,162 (L/mol·cm) |
| Target Sequence: | Target Sequence: |
| GAUGCAAAUAGGCGUUAAU | CCAGAAAUGCCAGCGCUAA |
| (SEQ ID: 99) | (SEQ ID: 100) |
| Lincode SMARTpool siRNA N-173526-03, | Lincode SMARTpool siRNA N-173526-04, |
| Gm16258 | Gm16258 |
| Mol. Wt. 13,459.9 (g/mol) | Mol. Wt. 13,459.8 (g/mol) |
| Ext. Coeff. 360,628 (L/mol·cm) | Ext. Coeff. 357,691 (L/mol·cm) |
| Target Sequence: | Target Sequence: |
| CAGAAGACACGCACGGAGA | GGAAGGAAGUCAGCGCCAA |
| (SEQ ID: 101) | (SEQ ID: 102) |

TABLE 7
Detailed information for the 4 custom siHOTAIR sequences designed by us. The
oligonucleotides were converted to a 2′-hydroxyl, annealed, and desalted
duplex. SB = custom designed.

| Custom siRNAs for Human HOTAIR: | Sequences and SEQ ID: |
|---|---|
| siHOTAIR SB1 | Sense Strand: |
| Length: 21 nucleotides | 5′-CCAAAGAGUCUGAUGUUUACA-3′ |
| Mol. Wt. 13, 378.1 (g/mol) | (SEQ ID: 103) |
| Ext. Coeff. 381, 186 (L/mol·cm) | Antisense Strand: |
| Target Sequence: | 5′-UAAACAUCAGACUCUUUGGGG-3′ |
| CCCCAAAGAGTCTGATGTTTACA (SEQ | (SEQ ID: 104) |
| ID NO: 115) | |
| (target position: 457-479) | |
| siHOTAIR SB2 | Sense Strand: |
| Length: 21 nucleotides | 5′-CAUAAACAAUAUAUCUGUUGG-3′ |
| Mol. Wt. 13, 373.1 (g/mol) | (SEQ ID: 105) |
| Ext. Coeff. 402, 101 (L/mol·cm) | Antisense Strand: |
| Target Sequence: | 5′-AACAGAUAUAUUGUUUAUGAG-3′ |
| CTCATAAACAATATATCTGTTGG (SEQ | (SEQ ID: 106) |
| ID NO: 116) | |
| (target position: 1156-1178) | |
| SiHOTAIR SB3 | Sense Strand: |
| Length: 21 nucleotides | 5′-CUCUAUAAUAUGCUUAUAUUA-3′ |
| Mol. Wt. 13, 256.0 (g/mol) | (SEQ ID: 107) |
| Ext. Coeff. 405, 216 (L/mol·cm) | Antisense Strand: |
| Target Sequence: | 5′-AUAUAAGCAUAUUAUAGAGUU-3′ |
| AACTCTATAATATGCTTATATTA (SEQ | (SEQ ID: 108) |
| ID NO: 117) | |
| (target position: 1700-1722) | |
| SiHOTAIR SB4 | Sense Strand: |
| Length: 21 nucleotides | 5′-GUGUAUAUAUAAUAAUGUAUU-3′ |
| Mol. Wt. 13, 264.0 (g/mol) | (SEQ ID: 109) |
| Ext. Coeff. 413, 849 (L/mol·cm) | Antisense Strand: |
| Target Sequence: | 5′-UACAUUAUUAUAUAUACACAA-3′ |
| TTGTGTATATATAATAATGTATT (SEQ | (SEQ ID: 110) |
| ID NO: 118) | |
| (target position: 2160-2182) | |

### Example 3

Preamble:

Given the findings documented in the present invention, we seek to identify compounds with an inhibitory effect on HOTAIR expressions and its downstream targets related to diabetic retinopathy and other conditions as outlined in this invention.

Experimental Approach:

Our experiments employ the following approaches:

1) Epigenetic drug library screening to identify compounds for inhibition of HOTAIR expression, and

2) chemical modulation of existing compounds,

For the first approach, an epigenetic drug library screening for small molecules is carried out to identify novel drug compounds that can suppress HOTAIR and its pathogenetic capabilities in the aforesaid conditions.

For the second approach, we have already identified certain epigenetic compounds (DZNep and 5-aza-dC) that can reduce HOTAIR expressions and its downstream targets. As such, we take these compounds as lead compounds and chemically modulate them to identify potentially new, non-toxic, and better-targeted compounds that can be used to block HOTAIR in the context of diabetes-induced vascularization.

3) Functional assessments using the novel identified drug compounds.

The compounds of the first and second approach are used to perform various cellular experiments that involve treating endothelial cells with various levels of glucose and appropriate concentrations of the novel compound(s) identified in approaches 1 and 2 above. Following the analyses of toxicity, the compound (s) are further assessed for the inhibitory effects on HOTAIR expressions and downstream mechanisms. To identify specific biologic significances, we perform functional assessments through angiogenesis assay.

Data from these experiments will identify specific compound(s) of interest which can be further tested in the preclinical models.

### Example 4

### Materials and Methods

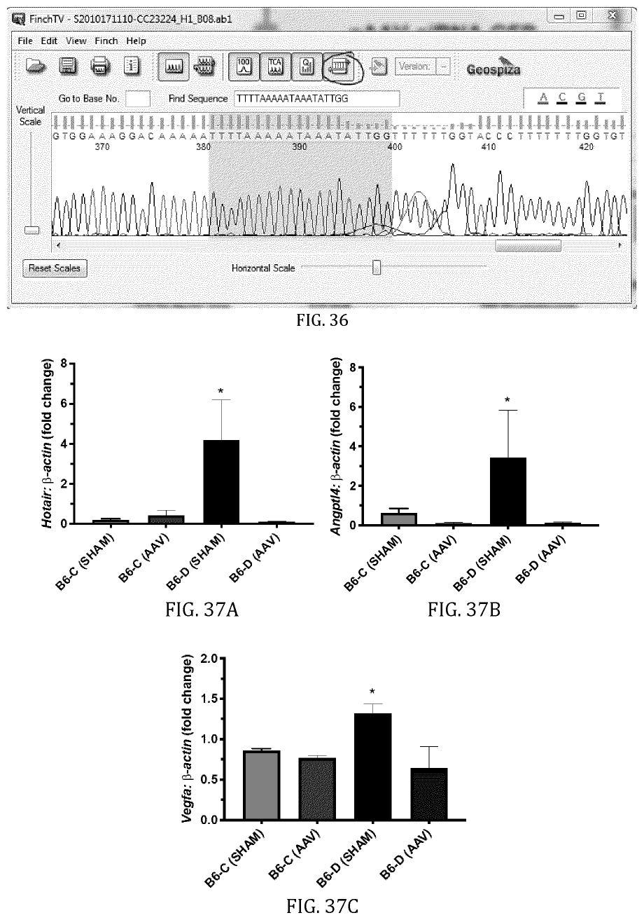

AAV-siRNA preparation: Custom duplex siRNA sequences targeting mouse Hotair (anti-sense strand=UUUUAAAAAUAAAUAUUGGAG (SEQ ID NO: 119); sense strand=CCAAUAUUUAUUUUUAAAAAA (SEQ ID NO: 120)) were reverse transcribed into DNA sequence and subsequently cloned into an AAV2 vector containing eGFP (enhanced green fluorescent protein), which was produced by Applied Biological Materials Inc. using proprietary methods (ABM; Richmond, British Columbia, Canada; general vector map shown in FIG. 35). Viral titers were determined as genomic copies (gc) per mL (1012 gc/mL) using the qPCR AAV Titer kit (G931) and sequences were confirmed using DNA sequencing by ABM (see FIG. 36).

### Animals:

Similar to the siRNA experiments, male mice (C57/BL6J background; 21.5±3.5 g; 8 weeks old) (Charles River Laboratories, Wilmington, Mass., USA) were used for the AAV-based experiments and randomly divided into control and diabetic groups (n=4/group). Streptozotocin (STZ) was used to generate a type 1 diabetic animal model. When the mice were ready for experimentation, a single dose of AAV (1 μL), with a titer of 1×10¹² gc/mL in PBS, was intravitreally injected into the left eyes of mice with or without diabetes (n=4). While right eyes received a sham injection of PBS (n=4) for comparison. Following the injection, all mice were monitored for 1 month and then euthanized for subsequent retinal tissue extraction and RT-qPCR analyses.

### Results

A single intravitreal administration of AAV-siHOTAIR was capable of reducing retinal Hotair RNA expressions by ˜97.3% in diabetic mice at 1 month when compared to Hotair levels from retinal tissues of diabetic mice treated with sham controls (PBS only; FIG. 37A). Decreased trends in Angptl4 (FIG. 37B) and Vegfa (FIG. 37C) expressions were also observed in the retinal tissues of diabetic mice at 1 month following the one-time injection of AAV-siHOTAIR.

### Example 5

### Materials and Methods

Undiluted serum samples were collected in BD gold-top serum separator tubes from human patients prior to a pars plana vitrectomy. Serum specimens were submitted to the research laboratory, where total RNA was extracted from 200 μL of serum samples using TRIzol reagent (Invitrogen) and a serum RNA extraction kit (Bio Basic Inc., Markham, ON, CAN) following the manufacturer's protocol. The expression levels of 9-specific lncRNAs (ANRIL, H19, HOTAIR, HULC, MALAT1, MEG3, MIAT, WISPER and ZFAS1) were assessed using RT-qPCR, where a research technician was blinded to the sample type (i.e., control versus diabetic retinopathy). Specimens were categorized into two groups: control (‘C’) and diabetic retinopathy (‘P’; DR). The ‘P’ group comprised of diabetic patients diagnosed with varying stages of DR (indicated as ‘0’=diabetic patients with no retinopathy, ‘1’=diabetic patients with non-proliferative DR [NPDR], and ‘2’=diabetic patients with proliferative DR [PDR]) (n=38; mean age ±SD=62.87±12.49 years; 32 males and 6 females), while the control group consisted of non-diabetic patients that had no previous history of DR and were diagnosed with idiopathic macular hole or a separate non-diabetic ocular condition (n=4; mean age ±SD=65.25±12.04 years; 1 male and 3 females). PDR was defined as the presence of neovascularization or fibrous proliferation of the disc or elsewhere on the retina.

### Statistics:

Statistical differences were evaluated between groups using GraphPad Prism 7 (La Jolla, Calif., USA) and Microsoft Excel (Washington, USA). Data were considered statistically significant if the p value was less than 0.05. Statistical significance for the clinical samples (with non-normal distribution) were identified using the Mann-Whitney U test (when comparing two conditions) or Kruskal-Wallis one-way ANOVA (for multiple group comparisons).

### Results

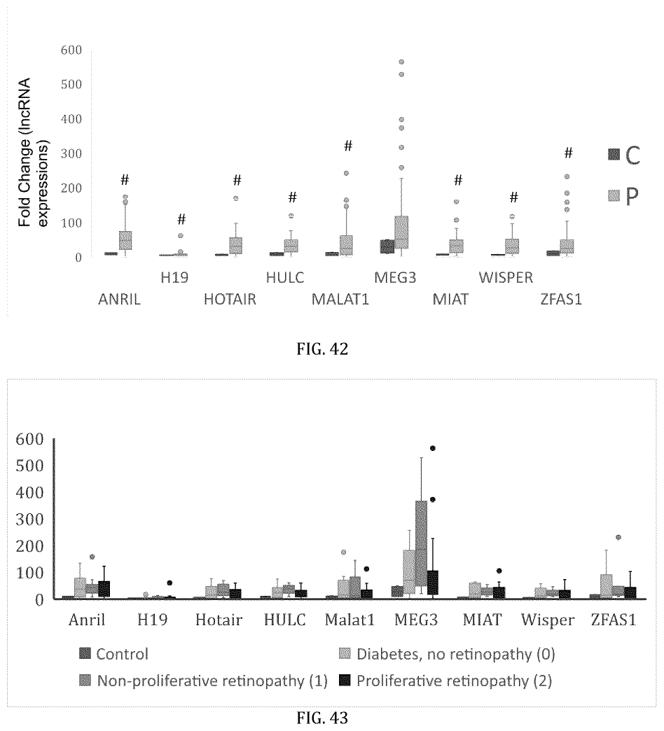

As shown by the box and whisker plots in FIG. 42, significant increased lncRNA expressions of ANRIL, H19, HOTAIR, HULC, MALAT1, MIAT, WISPER and ZFAS1 were observed in the serum of diabetic patients (with varying stages of DR; group ‘P’) compared to non-diabetic patients (without DR; group ‘C’). Although not significant, MEG3 lncRNA levels also demonstrated increasing trends in diabetic patients compared to control patients (p=0.063). Furthermore, creatinine and hemoglobin A1c data points were collected from patients in both ‘C’ and ‘P’ groups and the data points were compared against each lncRNA expression values. No significant correlations were demonstrated between the lncRNA expression profiles and creatinine or hemoglobin A1c levels (data not shown).

‘P’ groups were further stratified into distinct sub-groups, where ‘0’ indicated patients with diabetes and no DR, ‘1’ indicated patients with diabetes and NPDR and ‘2’ indicated patients with diabetes and PDR. As shown in FIG. 43, the average expression levels for the 9 lncRNAs were generally increased across all ‘P’ sub-groups (‘0’, ‘1’, and ‘2’) when compared to the lncRNA expression profiles from the control group. Furthermore, lncRNA expression levels from each patient sub-group were compared against the lncRNA expression levels from the control group. In particular, when comparing between control and ‘0’ groups, significances were only observed for the lncRNA H19, while the remaining 8 lncRNAs did not demonstrate statistical significance (Table 8). When the lncRNAs in the control group were compared against the lncRNAs from sub-group ‘1’ (diabetic patients with NPDR), significances were observed for all lncRNAs. Additionally, comparing the lncRNA expression values between control and group ‘2’ (diabetic patients with PDR) also demonstrated statistical significance for ANRIL, H19, HOTAIR, HULC, MALAT1, MIAT, and WISPER, while MEG3 and ZFAS1 did not show significance. Collectively, the results suggest that these 9 lncRNAs can be used as biomarkers of DR.

Based on the ROC curve analysis (Table 9), area under the curve (AUC) demonstrated that the lncRNAs of interest have good diagnostic value in discriminating diabetic patient sub-groups (diabetes, NPDR, and PDR) from control non-diabetic patients. Specifically, using AUC, comparison between control patients and diabetic patients with no DR demonstrated a significant difference in all 9 lncRNAs, where HULC had the highest AUC (AUC=0.85, p<0.001) and 8 out of the 9 lncRNAs had AUC values greater than 0.7 (with the exception of H19; AUC=0.68). Furthermore, when analyzing the AUC values for control patients and NPDR patients (group ‘1’), all 9 lncRNAs demonstrated statistical significance and 6 out of 9 lncRNAs had AUC values between 0.8 to 0.89, with the exception of ANRIL (AUC=0.90, p<0.001), MALAT1 (AUC=0.97, p<0.001), and ZFAS1 (AUC=0.94, p<0.001). Additionally, comparison of AUC values between control patients and PDR patients (group ‘2’) demonstrated significance for all of the lncRNA markers and 7 out of the 9 lncRNAs had AUC values greater than 0.9, with the exception of HOTAIR (AUC=0.89, p<0.001) and ZFAS1 (AUC=0.83, p<0.001). These results demonstrate that the 9 lncRNAs examined can be used as a prognostic tool for discriminate between non-diabetic and diabetic patients with DR. These lncRNAs may also serve to discriminate various stages of DR (mild NPDR to severe PDR) from diabetic patients without DR.

TABLE 8
P values comparing the IncRNA expressions in the control group
against the IncRNA expressions in each ‘P’ sub-group (‘0’, ‘1’ or ‘2’).

| | p-values for FIG. 43 | | |
|---|---|---|---|
| | Control versus | Control versus | Control versus |
| IncRNAs | Diabetic (0) | NPDR (1) | PDR (2) |
| ANRIL | 0.087 | 9E−05 | 5E−05 |
| H19 | 0.021 | 0.003 | 6E−04 |
| HOTAIR | 0.248 | 9E−05 | 3E−04 |
| HULC | 0.087 | 5E−05 | 2E−05 |
| MALAT1 | 0.117 | 0.009 | 0.021 |
| MEG3 | 0.154 | 0.001 | 0.063 |
| MIAT | 0.063 | 2E−05 | 6E−04 |
| WISPER | 0.063 | 2E−05 | 0.002 |
| ZFAS1 | 0.198 | 0.005 | 0.063 |

Legend:
‘0’ = diabetic patients with no DR,
‘1’ = diabetic patients with non-proliferative DR, and
‘2’ = diabetic patients with proliferative DR.

TABLE 9
Area under the curve (AUC) values for lncRNA expressions between control patients and
each ‘P’ sub-group (‘0’, ‘1’, or ‘2’).

| | | A | H19 | H | HULC | M | MEG3 | MIAT | W | Z |
|---|---|---|---|---|---|---|---|---|---|---|
| Control | AUC | 0.83 | 0.68 | 0.79 | 0.85 | 0.75 | 0.81 | 0.82 | 0.77 | 0.76 |
| versus | SE | 0.14 | 0.18 | 0.15 | 0.14 | 0.16 | 0.15 | 0.14 | 0.16 | 0.16 |
| ‘0’ | P-value | 1.6E−08 | 1.2E−04 | 3.7E−07 | 1.6E−09 | 7.5E−06 | 7.9E−08 | 2.0E−08 | 2.2E−06 | 4.6E−06 |
| Control | AUC | 0.90 | 0.82 | 0.89 | 0.88 | 0.97 | 0.89 | 0.86 | 0.82 | 0.94 |
| versus | SE | 0.11 | 0.14 | 0.12 | 0.12 | 0.07 | 0.12 | 0.13 | 0.14 | 0.09 |
| ‘1’ | P-value | 3.6E−15 | 1.1E−08 | 1.3E−13 | 2.0E−12 | 1.8E−43 | 1.2E−13 | 9.2E−11 | 1.2E−08 | 3.1E−26 |
| Control | AUC | 0.92 | 0.95 | 0.89 | 0.92 | 0.90 | 0.90 | 0.92 | 0.90 | 0.83 |
| versus | SE | 0.10 | 0.08 | 0.11 | 0.10 | 0.11 | 0.11 | 0.10 | 0.11 | 0.13 |
| ‘2’ | P-value | 9.4E−20 | 8.8E−30 | 7.0E−15 | 4.0E−19 | 3.0E−16 | 1.0E−15 | 9.8E−20 | 1.6E−15 | 1.9E−09 |

Legend: ‘0’ = diabetic patients with no DR, ‘1’ = diabetic patients with non-proliferative DR, and ‘2’ = diabetic patients with proliferative DR. “A”: ANRIL; “H”: HOTAIR; “M”: MALAT1; “W”: WISPER; “Z”: ZFAS1

### REFERENCES

1. Cho, N. H., Shaw, J. E., Karuranga, S., Huang, Y., da Rocha Fernandes, J. D., Ohlrogge, A. W., & Malanda, B. (2018). IDF Diabetes Atlas: Global estimates of diabetes prevalence for 2017 and projections for 2045. Diabetes Research and Clinical Practice, 138, 271-281. http://doi.org/10.1016/j.diabres.2018.02.023
2. Biswas, S., & Chakrabarti, S. (2017). Pathogenetic Mechanisms in Diabetic Retinopathy: From Molecules to Cells to Tissues. In Mechanisms of Vascular Defects in Diabetes Mellitus (pp. 209-247). http://doi.org/10.1007/978-3-319-60324-7_9
3. Nentwich, M. M. (2015). Diabetic retinopathy—ocular complications of diabetes mellitus. World Journal of Diabetes, 6(3), 489. http://doi.org/10.4239/wjd.v6.i3.489
4. Garg, S., & Davis, R. M. (2009). Diabetic retinopathy screening update. Clinical Diabetes. http://doi.org/10.2337/diaclin.27.4.140
5. Arroyo, A. G., & Iruela-Arispe, M. L. (2010). Extracellular matrix, inflammation, and the angiogenic response. Cardiovascular Research. http://doi.org/10.1093/cvr/cvq049
6. Grant, M. B., Afzal, A., Spoerri, P., Pan, H., Shaw, L. C., & Mames, R. N. (2004). The role of growth factors in the pathogenesis of diabetic retinopathy. Expert Opinion on Investigational Drugs. http://doi.org/10.1517/13543784.13.10.1275
7. Levy, A. P., Levy, N. S., Wegner, S., & Goldberg, M. A. (1995). Transcriptional regulation of the rat vascular endothelial growth factor gene by hypoxia. Journal of Biological Chemistry, 270(22), 13333-13340. http://doi.org/10.1074/jbc.270.22.13333
8. Wang, Y., Zang, Q. S., Liu, Z., Wu, Q., Maass, D., Dulan, G., . . . Nwariaku, F. E.

(2011). Regulation of vegf-induced endothelial cell migration by mitochondrial reactive oxygen species. American Journal of Physiology—Cell Physiology, 301(3). http://doi.org/10.1152/ajpce11.00322.2010
9. Okamoto, T., Yamagishi, S. ichi, Inagaki, Y., Amano, S., Koga, K., Abe, R., . . . Makita, Z. (2002). Angiogenesis induced by advanced glycation end products and its prevention by cerivastatin. The FASEB Journal: Official Publication of the Federation of American Societies for Experimental Biology, 16(14), 1928-1930. http://doi.org/10.1096/fj.02-0030fje
10. Angelo, L. S., & Kurzrock, R. (2007). Vascular endothelial growth factor and its relationship to inflammatory mediators. Clinical Cancer Research. http://doi.org/10.1158/1078-0432.CCR-06-2416
11. Carmeliet, P., & Jain, R. K. (2011). Molecular mechanisms and clinical applications of angiogenesis. Nature. http://doi.org/10.1038/nature10144
12. Turunen, M. P., Lehtola, T., Heinonen, S. E., Assefa, G. S., Korpisalo, P., Girnary, R., . . . Ylä-Herttuala, S. (2009). Efficient regulation of VEGF expression by promoter-targeted lentiviral shRNAs based on epigenetic mechanism a novel example of epigenetherapy. Circulation Research, 105(6), 604-609. http://doi.org/10.1161/ORCRESAHA.109.200774
13. Van Wijngaarden, P., Coster, D. J., & Williams, K. A. (2005). Inhibitors of ocular neovascularization: Promises and potential problems. Journal of the American Medical Association. http://doi.org/10.1001/jama.293.12.1509
14. Falavarjani, K. G., & Nguyen, Q. D. (2013). Adverse events and complications associated with intravitreal injection of anti-VEGF agents: A review of literature. Eye (Basingstoke). http://doi.org/10.1038/eye.2013.107
15. Duh, E. J., Sun, J. K., & Stitt, A. W. (2017). Diabetic retinopathy: current understanding, mechanisms, and treatment strategies. JCI Insight. http://doi.org/10.1172/jci.insight.93751
16. Bouckenheimer, J., Assou, S., Riquier, S., Hou, C., Philippe, N., Sansac, C., . . . De Vos, J. (2016). Long non-coding RNAs in human early embryonic development and their potential in ART. Human Reproduction Update, 23(1), 19-40. http://doi.org/10.1093/humupd/dmw035
17. Prensner, J. R., & Chinnaiyan, A. M. (2011). The emergence of lncRNAs in cancer biology. Cancer Discovery. http://doi.org/10.1158/2159-8290. CD-11-0209
18. Wang, D. Q., Fu, P., Yao, C., Zhu, L. S., Hou, T. Y., Chen, J. G., . . . Zhu, L. Q. (2018). Long Non-coding RNAs, Novel Culprits, or Bodyguards in Neurodegenerative Diseases. Molecular Therapy—Nucleic Acids, 10, 269-276. http://doi.org/10.1016/j.omtn.2017.12.011
19. Rinn, J. L., Kertesz, M., Wang, J. K., Squazzo, S. L., Xu, X., Brugmann, S. A., . . . Chang, H. Y. (2007). Functional Demarcation of Active and Silent Chromatin Domains in Human HOX Loci by Noncoding RNAs. Cell, 129(7), 1311-1323. http://doi.org/10.1016/j.cell.2007.05.022
20. Gupta, N., Mansoor, S., Sharma, A., Sapkal, A., Sheth, J., Falatoonzadeh, P., . . . Kenney, M. (2013). Diabetic Retinopathy and VEGF. The Open Ophthalmology Journal, 7(1), 4-10. http://doi.org/10.2174/1874364101307010004
21. Lam, H.-C., Lee, J.-K., Lu, C.-C., Chu, C.-H., Chuang, M.-J., & Wang, M.-C. (2005). Role of Endothelin in Diabetic Retinopathy. Current Vascular Pharmacology, 1(3), 243-250. http://doi.org/10.2174/1570161033476600
22. Biswas, S., Feng, B., Thomas, A., Chen, S., Aref-Eshghi, E., Sadikovic, B., & Chakrabarti, S. (2018). Endothelin-1 regulation is entangled in a complex web of epigenetic mechanisms in diabetes. Physiological Research, 67, S115-S125. http://doi.org/10.33549/physiolres.933836
23. Babapoor-Farrokhran, S., Jee, K., Puchner, B., Hassan, S. J., Xin, X., Rodrigues, M., . . . Sodhi, A. (2015). Angiopoietin-like 4 is a potent angiogenic factor and a novel therapeutic target for patients with proliferative diabetic retinopathy. Proceedings of the National Academy of Sciences of the United States of America, 112(23), E3030-E3039. http://doi.org/10.1073/pnas.1423765112
24. Khaliq, A., Foreman, D., Ahmed, A., Weich, H., Gregor, Z., McLeod, D., & Boulton, M. (1998). Increased expression of placenta growth factor in proliferative diabetic retinopathy. Laboratory Investigation, 78(1), 109-116.
25. Dinarello, C. A. (2011). Interleukin-1 in the pathogenesis and treatment of inflammatory diseases. Blood, 117(14), 3720-3732. http://doi.org/10.1182/blood-2010-07-273417
26. Carmi, Y., Dotan, S., Rider, P., Kaplanov, I., White, M. R., Baron, R., . . . Voronov, E. (2013). The Role of IL-1β in the Early Tumor Cell-Induced Angiogenic Response. The Journal of Immunology, 190(7), 3500-3509. http://doi.org/10.4049/jimmunol.1202769
27. Biswas, S., Thomas, A. A., Chen, S., Aref-Eshghi, E., Feng, B., Gonder, J., . . . Chakrabarti, S. (2018). MALAT1: An Epigenetic Regulator of Inflammation in Diabetic Retinopathy. Scientific Reports, 8(1). http://doi.org/10.1038/s41598-018-24907-w
28. Zhou, J., Wang, S., & Xia, X. (2012). Role of intravitreal inflammatory cytokines and angiogenic factors in proliferative diabetic retinopathy. Current Eye Research, 37(5), 416-420. http://doi.org/10.3109/02713683.2012.661114
29. Kowluru, R. A., & Odenbach, S. (2004). Role of interleukin-113 in the pathogenesis of diabetic retinopathy. British Journal of Ophthalmology, 88(10), 1343-1347. http://doi.org/10.1136/bjo.2003.038133
30. Wang, X., Wang, G., & Wang, Y. (2009). Intravitreous Vascular Endothelial Growth Factor and Hypoxia-Inducible Factor 1a in Patients With Proliferative Diabetic Retinopathy. American Journal of Ophthalmology, 148(6), 883-889. http://doi.org/10.1016/j.ajo.2009.07.007
31. Obrosova, I. G., Li, F., Abatan, O. I., Forsell, M. A., Komjáti, K., Pacher, P., . . . Stevens, M. J. (2004). Role of Poly(ADP-Ribose) Polymerase Activation in Diabetic Neuropathy. Diabetes, 53(3), 711-720. http://doi.org/10.2337/diabetes.53.3.711
32. Mishra, M., & Kowluru, R. A. (2017). Role of PARP-1 as a novel transcriptional regulator of MMP-9 in diabetic retinopathy. Biochimica et Biophysica Acta—Molecular Basis of Disease, 1863(7), 1761-1769. http://doi.org/10.1016/j.bbadis.2017.04.024
33. Liu, J., Chen, S., Biswas, S., Nagrani, N., Chu, Y., Chakrabarti, S., & Feng, B. (2020). Glucose-induced oxidative stress and accelerated aging in endothelial cells are mediated by the depletion of mitochondrial SIRTs. Physiological Reports, 8(3). http://doi.org/10.14814/phy2.14331
34. Al-Kafaji, G., Sabry, M. A., & Bakhiet, M. (2016). Increased expression of mitochondrial DNA-encoded genes in human renal mesangial cells in response to high glucose-induced reactive oxygen species. Molecular Medicine Reports, 13(2), 1774-1780. http://doi.org/10.3892/mmr.2015.4732
35. Hirata, H.; Hinoda, Y.; Shahryari, V.; Deng, G.; Nakajima, K.; Tabatabai, Z. L.; Ishii, N.; Dahiya, R. Long noncoding RNA MALAT1 promotes aggressive renal cell carcinoma through Ezh2 and interacts with miR-205. Cancer Res. 2015, 75, 1322-1331.
36. Ren, S.; Liu, Y.; Xu, W.; Sun, Y.; Lu, J.; Wang, F.; Wei, M.; Shen, J.; Hou, J.; Gao, X.; et al. Long noncoding RNA MALAT-1 is a new potential therapeutic target for castration resistant prostate cancer. J. Urol. 2013, 190, 2278-2287.
37. Zhang, Q. S.; Wang, Z. H.; Zhang, J. L.; Duan, Y. L.; Li, G. F.; Zheng, D. L. Beta-asarone protects against MPTP-induced Parkinson's disease via regulating long non-coding RNA MALAT1 and inhibiting α-synuclein protein expression. Biomed. Pharmacother. 2016, 83, 153-159.
38. Vausort, M.; Wagner, D. R.; Devaux, Y. Long noncoding RNAs in patients with acute myocardial infarction. Circ. Res. 2014, 115, 668-677.
39. Pachnis, V.; Belayew, A.; Tilghman, S. M. Locus unlinked to alpha-fetoprotein under the control of the murine raf and Rif genes. Proc. Natl. Acad. Sci. USA 2006, 81, 5523-5527.
40. Micheletti, R. et al., 2017. The long noncoding RNA Wisper controls cardiac fibrosis and remodeling. Science Translational Medicine, 9(395).
41. Askarian-Amiri M E, Crawford J, Franch J D, Smart C E, Smith M A, Clark M B, Ru K, M T M, Thompson E R, Lakhani S R, Vargas A C, Campbell I G, Brown M A, Dinger M E, Mattick J S. SNORD-host RNA Zfas1 is a regulator of mammary development and a potential marker for breast cancer. RNA., 2011. 17:878-891.
42. Faltejskova-Vychytilova P, Hombach S, Mlcochova J, Kretz M, Svoboda M, Slaby O. Long non-coding RNA ZFAS1 interacts with CDK1 and is involved in p53-dependent cell cycle control and apoptosis in colorectal cancer. Oncotarget., 2016. 7:622-637.
43. Panzitt K, Tschernatsch M M, Guelly C, Moustafa T, Stradner M, Strohmaier H M, Buck CR, Denk H, Schroeder R, Trauner M, Zatloukal K. Characterization of HULC, a novel gene with striking up-regulation in hepatocellular carcinoma, as noncoding RNA. Gastroentology, 2007. 132:330-342.
44. Ishii, N.; Ozaki, K.; Sato, H.; Mizuno, H.; Saito, S.; Takahashi, A.; Miyamoto, Y.; Ikegawa, S.; Kamatani, N.; Hori, M.; et al. Identification of a novel non-coding RNA, MIAT, that confers risk of myocardial infarction. J. Hum. Genet. 2006, 51, 1087-1099.
45. Barry, G.; Briggs, J. A.; Vanichkina, D. P.; Poth, E. M.; Beveridge, N. J.; Ratnu, V. S.; Nayler, S. P.; Nones, K.; Hu, J.; Bredy, T. W.; et al. The long non-coding RNA Gomafu is acutely regulated in response to neuronal activation and involved in schizophrenia-associated alternative splicing. Mol. Psychiatry 2014, 19, 486-494.
46. Zhang, H. Y.; Zheng, F. S.; Yang, W.; Lu, J. Bin. The long non-coding RNA MIAT regulates zinc finger E-box binding homeobox 1 expression by sponging miR-150 and promoteing cell invasion in non-small-cell lung cancer. Gene 2017, 633, 61-65.
47. Blackshaw, S.; Harpavat, S.; Trimarchi, J.; Cai, L.; Huang, H.; Kuo, W. P.; Weber, G.; Lee, K.; Fraioli, R. E.; Cho, S. H.; et al. Genomic analysis of mouse retinal development. PLoS Biol. 2004, 2, 1-21.
48. Aprea, J.; Prenninger, S.; Dori, M.; Ghosh, T.; Monasor, L. S.; Wessendorf, E.; Zocher, S.; Massalini, S.; Alexopoulou, D.; Lesche, M.; et al. Transcriptome sequencing during mouse brain development identifies long non-coding RNAs functionally involved in neurogenic commitment. EMBO J. 2013, 32, 3145-3160.
49. Shen, Y.; Dong, L. F.; Zhou, R. M.; Yao, J.; Song, Y. C.; Yang, H.; Jiang, Q.; Yan, B. Role of long non-coding RNA MIAT in proliferation, apoptosis and migration of lens epithelial cells: A clinical and in vitro study. J. Cell Mol. Med. 2016, 20, 537-548.
50. Pasmant, E.; Laurendeau, I.; Heron, D.; Vidaud, M.; Vidaud, D.; Biéche, I. Characterization of a germ-line deletion, including the entire INK4/ARF locus, in a melanoma-neural system tumor family: Identification of ANRIL, an antisense noncoding RNA whose expression coclusters with ARF. Cancer Res. 2007, 67, 3963-3969.
51. Broadbent, H. M.; Peden, J. F.; Lorkowski, S.; Goel, A.; Ongen, H.; Green, F.; Clarke, R.; Collins, R.; Franzosi, M. G.; Tognoni, G.; et al. Susceptibility to coronary artery disease and diabetes is encoded by distinct, tightly linked SNPs in the ANRIL locus on chromosome 9p. Hum. Mol. Genet. 2008, 17, 806-814.
52. Li, Z.; Yu, X.; Shen, J. ANRIL: A pivotal tumor suppressor long non-coding RNA in human cancers. Tumor Biol. 2016, 37, 5657-5661.
53. da Rocha, S. T.; Edwards, C. A.; Ito, M.; Ogata, T.; Ferguson-Smith, A. C. Genomic imprinting at the mammalian Dlk1-Dio3 domain. Trends Genet. 2008, 24, 306-316.
54. Zhou, Y.; Zhang, X.; Klibanski, A. MEG3 noncoding RNA: A tumor suppressor. J. Mol. Endocrinol. 2012, 48, 1-16.
55. Ruiz, M. A., Feng, B., & Chakrabarti, S. (2015). Polycomb repressive complex 2 regulates MiR-200b in retinal endothelial cells: Potential relevance in diabetic retinopathy. PLoS ONE, 10(4). http://doi.org/10.1371/journal.pone.0123987
56. Thomas, A. A., Biswas, S., Feng, B., Chen, S., Gonder, J., & Chakrabarti, S. (2019). lncRNA H19 prevents endothelial-mesenchymal transition in diabetic retinopathy. Diabetologia, 62(3), 517-530. http://doi.org/10.1007/s00125-018-4797-6
57. Thomas, A. A., Feng, B., & Chakrabarti, S. (2017). ANRIL: A regulator of VEGF in diabetic retinopathy. Investigative Ophthalmology and Visual Science, 58(1), 470-480. http://doi.org/10.1167/iovs.16-20569
58. Merchan, J. R., Kovács, K., Railsback, J. W., Kurtoglu, M., Jing, Y., Piña, Y., . . . Lampidis, T. J. (2010). Antiangiogenic activity of 2-deoxy-D-glucose. PLoS ONE, 5(10). http://doi.org/10.1371/journal.pone.0013699
59. Kungulovski, G., Nunna, S., Thomas, M., Zanger, U. M., Reinhardt, R., & Jeltsch, A. (2015). Targeted epigenome editing of an endogenous locus with chromatin modifiers is not stably maintained. Epigenetics and Chromatin, 8(1). http://doi.org/10.1186/s13072-015-0002-z
60. Chen, S., Apostolova, M. D., Cherian, M. G., & Chakrabarti, S. (2000). Interaction of endothelin-1 with vasoactive factors in mediating glucose-induced increased permeability in endothelial cells. Laboratory Investigation, 80(8), 1311-1321. http://doi.org/10.1038/labinvest.3780139
61. Aref-Eshghi E, Biswas S, Chen C, Sadikovic B, Chakrabarti S. (2020). Glucose-induced duration-dependent genome-wide DNA methylation changes in human endothelial cells. Am J Physiol Cell Physiol. Published online ahead of print, 2020 May 27, 1-20. doi:10.1152/ajpcell.00011.2020
62. Turchinovich, A., Zoidl, G., & Dermietzel, R. (2010). Non-viral siRNA delivery into the mouse retina in vivo. BMC Ophthalmology, 10(1). http://doi.org/10.1186/1471-2415-10-25
63. Clark, M. B., Johnston, R. L., Inostroza-Ponta, M., Fox, A. H., Fortini, E., Moscato, P., . . . Mattick, J. S. (2012). Genome-wide analysis of long noncoding RNA stability. Genome Research, 22(5), 885-898. http://doi.org/10.1101/gr.131037.111
64. Qazi, Y., Maddula, S., & Ambati, B. K. (2009). Mediators of ocular angiogenesis. Journal of Genetics. http://doi.org/10.1007/s12041-009-0068-0
65. Pacher, P., & Szabó, C. (2005). Role of poly(ADP-ribose) polymerase-1 activation in the pathogenesis of diabetic complications: Endothelial dysfunction, as a common underlying theme. Antioxidants and Redox Signaling. http://doi.org/10.1089/ars.2005.7.1568
66. Li, L., Liu, B., Wapinski, O. L., Tsai, M. C., Qu, K., Zhang, J., . . . Chang, H. Y. (2013).
Targeted Disruption of Hotair Leads to Homeotic Transformation and Gene Derepression. Cell Reports, 5(1), 3-12. http://doi.org/10.1016/j.celrep.2013.09.003
67. Zheng, P., Xiong, Q., Wu, Y., Chen, Y., Chen, Z., Fleming, J., . . . Ge, F. (2015). Quantitative proteomics analysis reveals novel insights into mechanisms of action of long noncoding RNA hox transcript antisense intergenic RNA (HOTAIR) in HeLa cells. Molecular and Cellular Proteomics, 14(6), 1447-1463. http://doi.org/10.1074/mcp.M114.043984
68. Hoffmann, S., Spitkovsky, D., Radicella, J. P., Epe, B., & Wiesner, R. J. (2004). Reactive oxygen species derived from the mitochondrial respiratory chain are not responsible for the basal levels of oxidative base modifications observed in nuclear DNA of mammalian cells. Free Radical Biology and Medicine, 36(6), 765-773. http://doi.org/10.1016/j.freeradbiomed.2003.12.019
69. Tan, J. Z., Yan, Y., Wang, X. X., Jiang, Y., & Xu, H. E. (2014). EZH2: Biology, disease, and structure-based drug discovery. Acta Pharmacologica Sinica. http://doi.org/10.1038/aps.2013.161
70. Hajjari, M., & Rahnama, S. (2017). HOTAIR Long Non-coding RNA: Characterizing the Locus Features by the In Silico Approaches. Genomics & Informatics, 15(4), 170-177. http://doi.org/10.5808/gi.2017.15.4.170
71. Cuddapah, S., Jothi, R., Schones, D. E., Roh, T. Y., Cui, K., & Zhao, K. (2009). Global analysis of the insulator binding protein CTCF in chromatin barrier regions reveals demarcation of active and repressive domains. Genome Research, 19(1), 24-32. http://doi.org/10.1101/gr.082800.108
72. Ong, C. T., & Corces, V. G. (2008). Modulation of CTCF Insulator Function by Transcription of a Noncoding RNA. Developmental Cell. http://doi.org/10.1016/j.devcel.2008.09.013
73. Li, H., An, J., Wu, M., Zheng, Q., Gui, X., Li, T., . . . Lu, D. (2015). LncRNA HOTAIR promotes human liver cancer stem cell malignant growth through downregulation of SETD2. Oncotarget, 6(29), 27847-27864. http://doi.org/10.18632/oncotarget.4443
74. Xie, M. Y., Yang, Y., Liu, P., Luo, Y., & Tang, S. B. (2019). 5-aza-2′-deoxycytidine in the regulation of antioxidant enzymes in retinal endothelial cells and rat diabetic retina. International Journal of Ophthalmology, 12(1), 1-7. http://doi.org/10.18240/ijo.2019.01.01
75. Miki, K., Shimizu, E., Yano, S., Tani, K., & Sone, S. (2000). Demethylation by 5-aza-2′-deoxycytidine (5-azadC) of p16INK4A gene results in downregulation of vascular endothelial growth factor expression in human lung cancer cell lines. Oncology Research, 12(8), 335-342. http://doi.org/10.3727/096504001108747783
76. Wan, J., Oliver, V. F., Wang, G., Zhu, H., Zack, D. J., Merbs, S. L., & Qian, J. (2015).
Characterization of tissue-specific differential DNA methylation suggests distinct modes of positive and negative gene expression regulation. BMC Genomics, 16(1). http://doi.org/10.1186/s12864-015-1271-4

Through the embodiments that are illustrated and described, the currently contemplated best mode of making and using the invention is described. Without further elaboration, it is believed that one of ordinary skill in the art can, based on the description presented herein, utilize the present invention to the full extent. All publications cited herein are incorporated by reference.

Although the description above contains many specificities, these should not be construed as limiting the scope of the invention, but as merely providing illustrations of some of the presently embodiments of this invention.

1. A method of identifying a subject having an increased likelihood of progressing to end-organ damage due to diabetes comprising: a) measuring the amount of a long non-coding RNA (lncRNA) in a biological sample derived from the subject; and b) comparing the amount of the lncRNA with a control reference value of said lncRNA, and when the amount of the lncRNA is altered relative to the control reference value, identifying the subject as having increased likelihood of progressing to end-organ damage due to diabetes, wherein the lncRNA is one or more of HOTAIR, H19, WISPER, ZFAS1, HULC, ANRIL, MALAT1, and MIAT, and wherein when the subject has increased likelihood of progressing to end-organ damage due to diabetes, the method further comprises administering to the subject an agent effective to treat the end-organ damage due to diabetes.

2. (canceled)

3. (canceled)

4. The method of claim 1, wherein the amount of lncRNA is measured by performing polymerase chain reaction (PCR) using at least one set of oligonucleotide primers comprising a forward primer and a reverse primer capable of amplifying a lncRNA polynucleotide sequence, wherein at least one set of primers selected from: when the subject is a human a forward primer comprising the sequence of SEQ ID NOs: 3, 77, 79, 81, 83, 85, 87, 89, 91 or 93 and a corresponding reverse primer comprising the sequence of SEQ ID NO: 4, 78, 80, 82, 84, 86, 88, 90, 92 or 94, and when the subject is a mouse a forward primer comprising the sequence of SEQ ID NO:43 and a reverse primer comprising the sequence of SEQ ID NO:44.

5. The method of claim 1, wherein the biological sample is serum or vitreous fluid.

6. The method of claim 1, wherein the end-organ damage due to diabetes is diabetic retinopathy (DR), and wherein when the subject is identified as having an increased likelihood of progressing to DR, the method further comprises administering to the subject an agent effective for treating DR.

7. (canceled)

8. The method of claim 6, wherein the agent effective for treating DR inhibits at least one biological activity of lncRNA HOTAIR.

9. The method of claim 6, wherein the amount of lncRNA is measured by performing polymerase chain reaction (PCR) using at least one set of oligonucleotide primers comprising a forward primer and a reverse primer capable of amplifying a lncRNA polynucleotide sequence, wherein at least one set of primers selected from: when the subject is a human a forward primer comprising the sequence of SEQ ID NOs: 3, 77, 79, 81, 83, 85, 87, 89, 91 or 93 and a corresponding reverse primer comprising the sequence of SEQ ID NO: 4, 78, 80, 82, 84, 86, 88, 90, 92 or 94, and when the subject is a mouse a forward primer comprising the sequence of SEQ ID NO:43 and a reverse primer comprising the sequence of SEQ ID NO:44.

10. The method of claim 6, wherein the biological sample is serum or vitreous fluid.

11. A method of treating a condition, the method comprising: administering to a subject in need thereof a therapeutically effective amount of at least one agent that inhibits at least one biological activity of the long non-coding RNA HOTAIR, wherein the condition is one or more of: diabetic retinopathy (DR), diabetic nephropathy, diabetic cardiomyopathy, diabetic neuropathy, proliferative vitreoretinopathies, neovascular glaucoma, ischemic retinopathy, retinopathy secondary to retinal vein occlusion, age-related macular degeneration, and intraocular tumours.

12. The method of treating a condition of claim 11, wherein the condition is DR.

13. (canceled)

14. The method of claim 11, wherein the subject is a subject that does not respond to anti-VEGF therapy.

15-20. (canceled)

21. The method of claim 1, wherein the subject is a human and the agent is siRNA, wherein the siRNA is SEQ ID NO:104, SEQ ID NO:106, SEQ ID NO:108, SEQ ID NO:110, N-187951-01, 187951-02, 187951-03, 187951-04.

22-24. (canceled)

25. The method of claim 1, wherein the agent is administered in combination with another therapeutic agent for treating the condition associated with diabetes-induced neovascularization.

26. An isolated siRNA selected from SEQ ID NOs: 104, 106, 108 and 110.

27-41. (canceled)

42. The method of claim 6, wherein the subject is a human and the agent is siRNA, wherein the siRNA is SEQ ID NO:104, SEQ ID NO:106, SEQ ID NO:108, SEQ ID NO:110, N-187951-01, 187951-02, 187951-03, 187951-04.

43. The method of claim 6, wherein the agent is administered in combination with another therapeutic agent for treating the condition associated with diabetes-induced neovascularization.

44. The method of claim 11, wherein the subject is a human and the agent is siRNA, wherein the siRNA is SEQ ID NO:104, SEQ ID NO:106, SEQ ID NO:108, SEQ ID NO:110, N-187951-01, 187951-02, 187951-03, 187951-04.

45. The method of claim 11, wherein the agent is administered in combination with another therapeutic agent for treating the condition associated with diabetes-induced neovascularization.


SEQUENCE LISTING (121 sequences)

SEQ ID NO: 1 - DNA, 20 bp, Artificial Sequence
  Synthetic
cctctatgccaacacagtgc

SEQ ID NO: 2 - DNA, 20 bp, Artificial Sequence
  Synthetic
catcgtactcctgcttgctg

SEQ ID NO: 3 - DNA, 23 bp, Artificial Sequence
  Synthetic
ggtagaaaaagcaaccacgaagc

SEQ ID NO: 4 - DNA, 25 bp, Artificial Sequence
  Synthetic
acataaacctctgtctgtgagtgcc

SEQ ID NO: 5 - DNA, 20 bp, Artificial Sequence
  Synthetic
gaactttctgctgtcttggg

SEQ ID NO: 6 - DNA, 20 bp, Artificial Sequence
  Synthetic
cttcgtgatgattctgccct

SEQ ID NO: 7 - DNA, 21 bp, Artificial Sequence
  Synthetic
aagccctccagagagcgttat

SEQ ID NO: 8 - DNA, 25 bp, Artificial Sequence
  Synthetic
ccgaaggtctgtctgtcaccaatgt

SEQ ID NO: 9 - DNA, 19 bp, Artificial Sequence
  Synthetic
ggacacggcctatagcctg

SEQ ID NO: 10 - DNA, 20 bp, Artificial Sequence
  Synthetic
ctcttggcgcagttcttgtc

SEQ ID NO: 11 - DNA, 22 bp, Artificial Sequence
  Synthetic
ccggctcgtgtatttattaccg

SEQ ID NO: 12 - DNA, 22 bp, Artificial Sequence
  Synthetic
ggcaaccactgttctccagagc

SEQ ID NO: 13 - DNA, 21 bp, Artificial Sequence
  Synthetic
gcggcatccagctacgaatct

SEQ ID NO: 14 - DNA, 20 bp, Artificial Sequence
  Synthetic
gggcagggaaccagcatctt

SEQ ID NO: 15 - DNA, 24 bp, Artificial Sequence
  Synthetic
cataaagtctgcaacatggaaggt

SEQ ID NO: 16 - DNA, 23 bp, Artificial Sequence
  Synthetic
atttgatgggtgaggaatgggtt

SEQ ID NO: 17 - DNA, 20 bp, Artificial Sequence
  Synthetic
ccacacacaatgcgtatgac

SEQ ID NO: 18 - DNA, 21 bp, Artificial Sequence
  Synthetic
ccacagcaatcttcggttatg

SEQ ID NO: 19 - DNA, 20 bp, Artificial Sequence
  Synthetic
tcaccagacgcctcaaccgc

SEQ ID NO: 20 - DNA, 20 bp, Artificial Sequence
  Synthetic
gcctcgcccgatgtgtagga

SEQ ID NO: 21 - DNA, 20 bp, Artificial Sequence
  Synthetic
tcgcctccagcatgaaagtc

SEQ ID NO: 22 - DNA, 19 bp, Artificial Sequence
  Synthetic
ggcattgattgcatctggc

SEQ ID NO: 23 - DNA, 21 bp, Artificial Sequence
  Synthetic
gaccccacccttcttcagatg

SEQ ID NO: 24 - DNA, 20 bp, Artificial Sequence
  Synthetic
ccacagcagcctctgcttct

SEQ ID NO: 25 - DNA, 20 bp, Artificial Sequence
  Synthetic
gggactaaccaatggtggtg

SEQ ID NO: 26 - DNA, 20 bp, Artificial Sequence
  Synthetic
attgggagaagtcaagcctg

SEQ ID NO: 27 - DNA, 20 bp, Artificial Sequence
  Synthetic
ccaccattaatgtgctggaa

SEQ ID NO: 28 - DNA, 21 bp, Artificial Sequence
  Synthetic
ttccttggaggagtatccaca

SEQ ID NO: 29 - DNA, 20 bp, Artificial Sequence
  Synthetic
tacggctcctattgccaaac

SEQ ID NO: 30 - DNA, 20 bp, Artificial Sequence
  Synthetic
tgcttcagtttgttgccttg

SEQ ID NO: 31 - DNA, 21 bp, Artificial Sequence
  Synthetic
gcaactgtaggaagcaacaga

SEQ ID NO: 32 - DNA, 21 bp, Artificial Sequence
  Synthetic
cataggtccatgcacaagtgt

SEQ ID NO: 33 - DNA, 20 bp, Artificial Sequence
  Synthetic
acggtgctcatgcttacaac

SEQ ID NO: 34 - DNA, 20 bp, Artificial Sequence
  Synthetic
ttagcctctccatcggactt

SEQ ID NO: 35 - DNA, 20 bp, Artificial Sequence
  Synthetic
ggcaaattctcagtggtgtg

SEQ ID NO: 36 - DNA, 20 bp, Artificial Sequence
  Synthetic
gtcactctcatcgctgctgt

SEQ ID NO: 37 - DNA, 20 bp, Artificial Sequence
  Synthetic
ttgaatatgaagcccccaag

SEQ ID NO: 38 - DNA, 20 bp, Artificial Sequence
  Synthetic
tgatattcccctcgtgcttc

SEQ ID NO: 39 - DNA, 20 bp, Artificial Sequence
  Synthetic
cagcctcctggtctgaactc

SEQ ID NO: 40 - DNA, 20 bp, Artificial Sequence
  Synthetic
atccaggggaagatctgctt

SEQ ID NO: 41 - DNA, 21 bp, Artificial Sequence
  Synthetic
atgtacatgccaccacctagc

SEQ ID NO: 42 - DNA, 21 bp, Artificial Sequence
  Synthetic
ttgctgtgtaacaggttgctc

SEQ ID NO: 43 - DNA, 20 bp, Artificial Sequence
  Synthetic
gcgccaacgtagaccaaaag

SEQ ID NO: 44 - DNA, 20 bp, Artificial Sequence
  Synthetic
tctaccgatgttggggacct

SEQ ID NO: 45 - DNA, 20 bp, Artificial Sequence
  Synthetic
atgcggatcaaacctcacca

SEQ ID NO: 46 - DNA, 21 bp, Artificial Sequence
  Synthetic
ctttctttggtctgcattcac

SEQ ID NO: 47 - DNA, 20 bp, Artificial Sequence
  Synthetic
ttagcaagaccatctgtgtg

SEQ ID NO: 48 - DNA, 20 bp, Artificial Sequence
  Synthetic
gagtttctccctgaaatgtg

SEQ ID NO: 49 - DNA, 20 bp, Artificial Sequence
  Synthetic
ttggtacctgtagccattcc

SEQ ID NO: 50 - DNA, 20 bp, Artificial Sequence
  Synthetic
gaggctaagaggctgctgta

SEQ ID NO: 51 - DNA, 20 bp, Artificial Sequence
  Synthetic
tgctgggaacaactcaacag

SEQ ID NO: 52 - DNA, 21 bp, Artificial Sequence
  Synthetic
cctcatcagggtattcatcca

SEQ ID NO: 53 - DNA, 21 bp, Artificial Sequence
  Synthetic
ttcaggcaggcagtatcactc

SEQ ID NO: 54 - DNA, 21 bp, Artificial Sequence
  Synthetic
gaaggtccacgggaaagacac

SEQ ID NO: 55 - DNA, 20 bp, Artificial Sequence
  Synthetic
tcaagtcagcaacgtggaag

SEQ ID NO: 56 - DNA, 20 bp, Artificial Sequence
  Synthetic
tatcgaggctgtgtcgactg

SEQ ID NO: 57 - DNA, 21 bp, Artificial Sequence
  Synthetic
ggaaagggatctactttgccg

SEQ ID NO: 58 - DNA, 20 bp, Artificial Sequence
  Synthetic
tcgggtctccctgagatgtg

SEQ ID NO: 59 - DNA, 20 bp, Artificial Sequence
  Synthetic
tccttcatgtcggacgaggc

SEQ ID NO: 60 - DNA, 21 bp, Artificial Sequence
  Synthetic
aatgctgtggctatgactgcg

SEQ ID NO: 61 - DNA, 22 bp, Artificial Sequence
  Synthetic
ttgtcaccaagctcaagagaga

SEQ ID NO: 62 - DNA, 22 bp, Artificial Sequence
  Synthetic
gaggtggttgtggaaaaggtag

SEQ ID NO: 63 - DNA, 20 bp, Artificial Sequence
  Synthetic
tggtccagatggcgtagagg

SEQ ID NO: 64 - DNA, 20 bp, Artificial Sequence
  Synthetic
gtcatcgagatccggctcag

SEQ ID NO: 65 - DNA, 20 bp, Artificial Sequence
  Synthetic
aggcagagtaggacagtgaa

SEQ ID NO: 66 - DNA, 20 bp, Artificial Sequence
  Synthetic
ctcagtctgggtcactcaat

SEQ ID NO: 67 - DNA, 23 bp, Artificial Sequence
  Synthetic
cgcgggactagggagtgttcagt

SEQ ID NO: 68 - DNA, 24 bp, Artificial Sequence
  Synthetic
agtacattataggcaccgaggcga

SEQ ID NO: 69 - DNA, 21 bp, Artificial Sequence
  Synthetic
agctctgccacagcaggttca

SEQ ID NO: 70 - DNA, 25 bp, Artificial Sequence
  Synthetic
tgcttttgttctttttggcctgcaa

SEQ ID NO: 71 - DNA, 23 bp, Artificial Sequence
  Synthetic
atgctgtcagtattgagagtggc

SEQ ID NO: 72 - DNA, 23 bp, Artificial Sequence
  Synthetic
gaggctgttcacacatttgaaag

SEQ ID NO: 73 - DNA, 20 bp, Artificial Sequence
  Synthetic
gagacctggcactgggaata

SEQ ID NO: 74 - DNA, 20 bp, Artificial Sequence
  Synthetic
tccaggggaagatctgtttg

SEQ ID NO: 75 - DNA, 23 bp, Artificial Sequence
  Synthetic
ataagcgcaacaaactcatttcg

SEQ ID NO: 76 - DNA, 20 bp, Artificial Sequence
  Synthetic
atatcgagggacgggaacct

SEQ ID NO: 77 - DNA, 23 bp, Artificial Sequence
  Synthetic
ggggcttccttgctcttcttatc

SEQ ID NO: 78 - DNA, 24 bp, Artificial Sequence
  Synthetic
ctgacactgaacggactctgtttg

SEQ ID NO: 79 - DNA, 20 bp, Artificial Sequence
  Synthetic
aaagacaccatcggaacagc

SEQ ID NO: 80 - DNA, 20 bp, Artificial Sequence
  Synthetic
agagtcgtcgaggctttgaa

SEQ ID NO: 81 - DNA, 20 bp, Artificial Sequence
  Synthetic
ccatctgtgggacatctgtg

SEQ ID NO: 82 - DNA, 20 bp, Artificial Sequence
  Synthetic
tgggggctgtggagatagta

SEQ ID NO: 83 - DNA, 18 bp, Artificial Sequence
  Synthetic
cagcgggtacagaatgga

SEQ ID NO: 84 - DNA, 22 bp, Artificial Sequence
  Synthetic
tcaggagatcgaaggttgtaga

SEQ ID NO: 85 - DNA, 21 bp, Artificial Sequence
  Synthetic
atctgcaagccaggaagagtc

SEQ ID NO: 86 - DNA, 22 bp, Artificial Sequence
  Synthetic
cttgcttgatgctttggtctgt

SEQ ID NO: 87 - DNA, 21 bp, Artificial Sequence
  Synthetic
cccttatttattcctggctcc

SEQ ID NO: 88 - DNA, 21 bp, Artificial Sequence
  Synthetic
gacctcgctttcctttcttcc

SEQ ID NO: 89 - DNA, 22 bp, Artificial Sequence
  Synthetic
tcttagagggtgggcttttgtt

SEQ ID NO: 90 - DNA, 22 bp, Artificial Sequence
  Synthetic
ctgcatctaggccatcatactg

SEQ ID NO: 91 - DNA, 22 bp, Artificial Sequence
  Synthetic
gggaggggaaatgggtgatgta

SEQ ID NO: 92 - DNA, 22 bp, Artificial Sequence
  Synthetic
taacgccaaatgtgaagtgtga

SEQ ID NO: 93 - DNA, 22 bp, Artificial Sequence
  Synthetic
cggctgggtcggctgaagaact

SEQ ID NO: 94 - DNA, 22 bp, Artificial Sequence
  Synthetic
ccgcccaaaccaggaaggagac

SEQ ID NO: 95 - RNA, 19 nt, Artificial Sequence
  Synthetic
agacgaaggugaaagcgaa

SEQ ID NO: 96 - RNA, 19 nt, Artificial Sequence
  Synthetic
caauauaucuguugggcgu

SEQ ID NO: 97 - RNA, 19 nt, Artificial Sequence
  Synthetic
gggacugggaggcgcuaau

SEQ ID NO: 98 - RNA, 19 nt, Artificial Sequence
  Synthetic
caguggaauggaacggauu

SEQ ID NO: 99 - RNA, 19 nt, Artificial Sequence
  Synthetic
gaugcaaauaggcguuaau

SEQ ID NO: 100 - RNA, 19 nt, Artificial Sequence
  Synthetic
ccagaaaugccagcgcuaa

SEQ ID NO: 101 - RNA, 19 nt, Artificial Sequence
  Synthetic
cagaagacacgcacggaga

SEQ ID NO: 102 - RNA, 19 nt, Artificial Sequence
  Synthetic
ggaaggaagucagcgccaa

SEQ ID NO: 103 - RNA, 21 nt, Artificial Sequence
  Synthetic
ccaaagagucugauguuuaca

SEQ ID NO: 104 - RNA, 21 nt, Artificial Sequence
  Synthetic
uaaacaucagacucuuugggg

SEQ ID NO: 105 - RNA, 21 nt, Artificial Sequence
  Synthetic
cauaaacaauauaucuguugg

SEQ ID NO: 106 - RNA, 21 nt, Artificial Sequence
  Synthetic
aacagauauauuguuuaugag

SEQ ID NO: 107 - RNA, 21 nt, Artificial Sequence
  Synthetic
cucuauaauaugcuuauauua

SEQ ID NO: 108 - RNA, 21 nt, Artificial Sequence
  Synthetic
auauaagcauauuauagaguu

SEQ ID NO: 109 - RNA, 21 nt, Artificial Sequence
  Synthetic
guguauauauaauaauguauu

SEQ ID NO: 110 - RNA, 21 nt, Artificial Sequence
  Synthetic
uacauuauuauauauacacaa

SEQ ID NO: 111 - DNA, 20 bp, Artificial Sequence
  Synthetic
gtagtcccagggtgcaacac

SEQ ID NO: 112 - DNA, 20 bp, Artificial Sequence
  Synthetic
gactggctagaatgggcatc

SEQ ID NO: 113 - DNA, 27 bp, Artificial Sequence
  Synthetic
cggtgctggaatttgatattcattgat

SEQ ID NO: 114 - DNA, 21 bp, Artificial Sequence
  Synthetic
ttcaagtggggaatggcaagc

SEQ ID NO: 115 - DNA, 23 bp, Artificial Sequence
  Synthetic
ccccaaagagtctgatgtttaca

SEQ ID NO: 116 - DNA, 23 bp, Artificial Sequence
  Synthetic
ctcataaacaatatatctgttgg

SEQ ID NO: 117 - DNA, 23 bp, Artificial Sequence
  Synthetic
aactctataatatgcttatatta

SEQ ID NO: 118 - DNA, 23 bp, Artificial Sequence
  Synthetic
ttgtgtatatataataatgtatt

SEQ ID NO: 119 - RNA, 21 nt, Artificial Sequence
  Synthetic
uuuuaaaaauaaauauuggag

SEQ ID NO: 120 - RNA, 21 nt, Artificial Sequence
  Synthetic
ccaauauuuauuuuuaaaaaa

SEQ ID NO: 121 - DNA, 60 bp, Artificial Sequence
  Synthetic
gaaggaaagccctccagcctccaggccctgccttctgcctgcacattctgccctgatttc